(19) United States
(12) Patent Application Publication
(10) Pub. No.: US 2011/0085970 A1
(43) Pub. Date: Apr. 14, 2011

(54) ANTI-B7H4 MONOCLONAL ANTIBODY-DRUG CONJUGATE AND METHODS OF USE

(76) Inventors: Jonathan A. Terrett, Sunnyvale, CA (US); Josephine M. Cardarelli, San Carlos, CA (US); Chetana Rao-Naik, Walnut Creek, CA (US); Bingliang Chen, Alameda, CA (US); David J. King, Solana Beach, CA (US)

(21) Appl. No.: 12/745,677

(22) PCT Filed: Nov. 26, 2008
(86) PCT No.: PCT/US2008/084923

§ 371 Date: Jun. 1, 2010

Related U.S. Application Data
(60) Provisional application No. 60/991,693, filed on Nov. 30, 2007.

(51) Int. Cl. A61K 51/10; C07K 16/00; A61K 39/395; A61P 35/00

(52) U.S. Cl. 424/1.49; 530/387.3; 424/133.1

(57) ABSTRACT

The present disclosure provides isolated monoclonal antibodies, particularly human monoclonal antibodies that specifically bind to B7H4 with high affinity. Nucleic acid molecules encoding the antibodies of this disclosure, expression vectors, host cells and methods for expressing the antibodies of this disclosure are also provided, immunoconjugates, including antibody-drug conjugates, bispecific molecules and pharmaceutical compositions comprising the antibodies of this disclosure are also provided. This disclosure also provides methods for treating cancer.

### FIELD OF THE INVENTION

The present invention provides anti-B7-H4 antibodies, antibody fragments, and antibody mimetics conjugated to partner molecules, such as drugs, radioisotopes, and toxins.

### BACKGROUND OF THE INVENTION

Breast and ovarian cancers are the second and fourth leading causes, respectively, of cancer deaths in females in the United States (American Cancer Society (2005) Cancer facts and figures). The American Cancer Society has estimated that, in the United States, approximately 40,000 women will die of breast cancer and about 16,000 will die of ovarian cancer in 2005. Surface epithelial tumors account for over 80% of all ovarian malignancies, which include serous tumors, mucinous tumors, endometrioid tumors and clear cell carcinomas (Seidman et al. “Blaustein's Pathology of the Female Genital Tract” 791-4 (Kurman, editor, 5th ed. New York, Springer-Verlag, 2002). Ovarian cancers frequently present at an advanced stage where metastatic disease has spread to regional and distant sites (Pettersson, (1994) Int. Fed. of Gyn. and Obstetrics, Vol. 22; and Heintz et al (2001) J. Epidermiol. Biostat. 6: 107-38). Thus, while the lifetime probability of developing breast cancer is significantly higher than for ovarian cancer, the 5 year survival rate for breast cancer patients is substantially better than for those with ovarian cancer.

B7-like molecules belong to the immunoglobulin (Ig) superfamily. The extracellular portions of B7-like molecules contain single IgV and IgC domains and share ˜20%-40% amino acid identity. B7-like molecules play critical roles in the control and fine tuning of antigen-specific immune responses. B7-H4, also known as O8E, B7x and B7S1, is a member of the B7 family and is thought to play a role in both stimulatory and inhibitory regulation of T cell responses (Carreno et al, (2002) Ann. Rev. Immunol. 20:29-53 and Khoury et al, (2004) Immunity 20:529-538). Human B7-H4 has been mapped on chromosome 1 and is comprised of six exons and five introns spanning 66 kb, of which exon 6 is used for alternative splicing to generate two different transcripts (Choi et al. (2003) J. Immunol. 171:4650-4654).

B7-H4 exerts its physiologic function by binding to a receptor on T cells, which in turn induces cell cycle arrest and inhibits the secretion, of cytokines, the development of cytotoxicity and cytokine production of CD4⁺ and CD8⁺ T cells (Prasad et al. (2003) Immunity 18:863-873; Sica et al. (2003) Immunity 18:849-861; Wang et al (2004) Microbes Infect. 6:759-66; and Zang et al. (2003) Proc. Natl. Acad. Sci. U.S.A. 100:10388-10392). It has been suggested that B7-H4 may be an attenuator of inflammatory responses and may serve a role in down-regulation of antigen-specific immune and anti-tumor responses (Zang et al. (2003) Proc. Natl. Acad. Sci. U.S.A. 100:10388-10392; Prasad et al (2003) Immunity 18:863-873; Sica et al. (2003) Immunity 18:849-861; Choi et al (2003) J. Immunol. 171:4650-4654; and Carreno et al. (2003) Trends Immunol. 24:524-7).

B7-H4 mRNA, but not protein, expression has been detected in a wide range of normal somatic tissues, including liver, skeletal muscle, kidney, pancreas and small bowel (Sica et al. (2003) Immunity 18:849-61 and Choi et al. (2003) J. Immunol. 171:4650-4). B7-H4 is inducible upon stimulation of T cells, B cells, monocytes and dendritic cells; however, immuno-histochemistry analysis has revealed little expression in several peripheral tissues with the exception of positive staining in some ovarian and lung cancers (Id.). In addition, B7-H4 is consistently overexpressed in primary and metastatic breast cancer, independent of tumor grade or stage, suggesting a critical role for this protein in breast cancer biology (Tringler et al. (2005) Clinical Cancer Res. U: 1842-48). See, also, U.S. Pat. Nos. 6,962,980; 6,699,664; 6,468,546; 6,488,931; 6,670,463; and 6,528,253, each of which is incorporated by reference herein in its entirety.

A wide variety of therapeutic modalities are available for the treatment of advanced breast and ovarian cancers including radiotherapy, conventional chemotherapy with cytotoxic antitumor agents, hormone therapy (aromatase inhibitors, luteinizing-hormone releasing-hormone analogues), bisphosphonates and signal-transduction inhibitors (Smith (2002) Lancet, 360:790-2). Unfortunately, however, many patients either respond poorly or not at all to any of these therapeutic modalities. Thus, there is a need to identify new molecular markers for and therapeutic agents against breast and ovarian cancers. Accordingly, B7-H4 represents a valuable target for the treatment of cancers, including ovarian and breast cancers and a variety of other diseases characterized by B7-H4 expression.

### SUMMARY OF THE INVENTION

The present disclosure provides antibody-partner molecule conjugates comprising monoclonal antibodies, in particular human sequence monoclonal antibodies, that bind to B7-H4 (a/k/a O8E, B7S1 and B7x) and that exhibit numerous desirable properties. These properties include high affinity binding to human B7-H4, internalization by cells expressing B7-H4, the ability to mediate antibody dependent cellular cytotoxicity, and/or the ability to inhibit growth of B7-H4-expressing cells in vivo when conjugated to a cytotoxin. Also provided are methods for treating a variety of B7-H4 mediated diseases using the antibody-partner molecule conjugates of this disclosure.

In one aspect, this disclosure pertains to antibody-partner molecule conjugates comprising a monoclonal antibody or an antigen-binding portion thereof, wherein the antibody:

(a) binds to human B7-H4 with an affinity of 1×10⁻⁸ M or less;

(b) is internalized by B7-H4-expressing cells;

(c) exhibits antibody dependent cellular cytotoxicity (ADCC) against B7-H4 expressing cells; and

(d) inhibits growth of B7-H4-expressing cells in vivo when conjugated to a cytotoxin.

Preferably, the antibody exhibits at least two of properties (a), (b), (c), and (d). More preferably, the antibody exhibits at least three of properties (a), (b), (c), and (d). More preferably, the antibody exhibits all four of properties (a), (b), (c), and (d). In another preferred embodiment, the antibody binds to B7-H4 with an affinity of 5×10⁻⁹ M or less. In yet another preferred embodiment, the antibody inhibits growth of B7-H4-expressing tumor cells in vivo when the antibody is conjugated to a cytotoxin.

In certain embodiments, the antibody binds to a breast cell carcinoma tumor cell line, such as cell line SKBR3 (ATCC Accession No. HTB-30).

Typically the antibody is a human antibody, although in alternative embodiments the antibody can be a murine antibody, a chimeric antibody or humanized antibody.

In another embodiment, the antibody is internalized by SKBR3 breast cell carcinoma tumor cells after binding to B7-H4 expressed on those cells.

In another embodiment, this disclosure provides an antibody-partner molecule conjugate comprising a monoclonal antibody or antigen binding portion thereof, wherein the antibody cross-competes for binding to B7-H4 with a reference antibody, wherein the reference antibody:

(a) a heavy chain variable region comprising the amino acid sequence of SEQ ID NO: 1 and a light chain variable region comprising the amino acid sequence of SEQ ID NO: 6;

(b) a heavy chain variable region comprising the amino acid sequence of SEQ ID NO: 2 and a light chain variable region comprising the amino acid sequence of SEQ ID NO: 7;

(c) a heavy chain variable region comprising the amino acid sequence of SEQ ID NO: 3 and a light chain variable region comprising the amino acid sequence of SEQ ID NO: 8;

(d) a heavy chain variable region comprising the amino acid sequence of SEQ ID NO: 4 and a light chain variable region comprising the amino acid sequence of SEQ TD NO: 9; or

(e) a heavy chain variable region comprising the amino acid sequence of SEQ ID NO: 5 and a light chain variable region comprising the amino acid sequence of SEQ ID NO: 10.

In one aspect, this disclosure pertains to an antibody-partner molecule conjugate comprising a monoclonal antibody or an antigen-binding portion thereof; comprising a heavy chain variable region that is the product of or derived from a human V_{H} 4-34 gene (the protein product of which is presented herein as SEQ ID NO: 51), wherein the antibody specifically binds B7-H4. This disclosure also provides an antibody-partner molecule conjugate comprising a monoclonal antibody or an antigen-binding portion thereof, comprising a heavy chain variable region that is the product of or derived from a human V_{H} 3-53 gene (the protein product of which is presented herein as SEQ ID NO: 52), wherein the antibody specifically binds B7-H4. This disclosure also provides an antibody-partner molecule conjugate comprising a monoclonal antibody or an antigen-binding portion thereof, comprising a heavy chain variable region that is the product of or derived from a combination of human V_{H} 3-9/D3-10/JH6b genes (the protein product of which is presented herein as SEQ ID NO: 53), wherein the antibody specifically binds B7-H4.

This disclosure still further provides an antibody-partner molecule conjugate comprising a monoclonal antibody or an antigen-binding portion thereof, comprising a light chain variable region that is the product of or derived from a human V_{K} A27 gene (the protein product of which is presented herein as SEQ ID NO: 54), wherein the antibody specifically binds B7-H4. This disclosure still further provides an antibody-partner molecule conjugate comprising a monoclonal antibody or an antigen-binding portion thereof, comprising a light chain variable region that is the product of or derived from a combination of human V_{K} L6/JK1 genes (the protein product of which is presented herein as SEQ ID NO: 55), wherein the antibody specifically binds B7-H4.

In other aspects, this disclosure provides an antibody-partner molecule conjugate comprising a monoclonal antibody or an antigen-binding portion thereof, comprising:

(a) a heavy chain variable region of a human V_{H} 4-34, 3-53 or 3-9 gene; and

(b) a light chain variable region of a human V_{K} A27 or VK L6; wherein the antibody specifically binds to B7-H4.

In a related embodiment, the antibody comprises a heavy chain variable region of a human V_{H} 4-34 gene and a light chain variable region of a human V_{K} A27 gene. In another related embodiment, the antibody comprises a heavy chain variable region of a human V_{H} 3-53 gene and a light chain variable region of a human V_{K} A27 gene. In yet another related embodiment, the antibody comprises a heavy chain variable region of a human V_{H} 3-9 gene and a light chain variable region of a human V_{K} L6 gene. In yet another aspect, the present disclosure provides an isolated monoclonal antibody or antigen binding portion thereof, comprising: a heavy chain variable region that comprises CDR1, CDR2 and CDR3 sequences; and a light chain variable region that comprises CDR1, CDR2 and CDR3 sequences, wherein:

(a) the heavy chain variable region CDR3 sequence comprises an amino acid sequence selected from the group consisting of amino acid sequences of SEQ ID NOs: 21, 22, 23, 24 and 25 and conservative modifications thereof;

(b) the light chain variable region CDR3 sequence comprises an amino acid sequence selected from the group consisting of amino acid sequence of SEQ ID NOs: 36, 37, 38, 39 and 40 and conservative modifications thereof;

(c) the antibody binds to human B7-H4 with a KD of 1×10⁻⁷ M or less;

(d) binds to human CHO cells transfected with B7-H4.

Preferably, the heavy chain variable region CDR2 sequence comprises an amino acid sequence selected from the group consisting of amino acid sequences of SEQ ID NOs: 16, 17, 18, 19 and 20 and conservative modifications thereof; and the light chain variable region CDR2 sequence comprises an amino acid sequence selected from the group consisting of amino acid sequences of SEQ ID NOs: 31, 32, 33, 34 and 35 and conservative modifications thereof.

Preferably, the heavy chain variable region CDR1 sequence comprises an amino acid sequence selected from the group consisting of amino acid sequences of SEQ ID NOs: 11, 12, 13, 14 and 15 and conservative modifications thereof; and the light chain variable region CDR1 sequence comprises an amino acid sequence selected from the group consisting of amino acid sequences of SEQ ID NOs: 26, 27, 28, 29 and 30 and conservative modifications thereof. A particular combination comprises:

(a) a heavy chain variable region CDR1 comprising SEQ ID NO: 11;

(b) a heavy chain variable region CDR2 comprising SEQ ID NO: 16;

(c) a heavy chain variable region CDR3 comprising SEQ ID NO: 21;

(d) a light chain variable region CDR1 comprising SEQ ID NO: 26;

(e) a light chain variable region CDR2 comprising SEQ ID NO: 31; and

(f) a light chain variable region CDR3 comprising SEQ ID NO: 36.

Another particular combination comprises:

(a) a heavy chain variable region CDR1 comprising SEQ ID NO: 12;

(b) a heavy chain variable region CDR2 comprising SEQ ID NO: 17;

(c) a heavy chain variable region CDR3 comprising SEQ ID NO: 22;

(d) a light chain variable region CDR1 comprising SEQ ID NO: 27;

(e) a light chain variable region CDR2 comprising SEQ ID NO: 32; and

(f) a light chain variable region CDR3 comprising SEQ ID NO: 37.

Another particular combination comprises:

(a) a heavy chain variable region CDR1 comprising SEQ ID NO: 13;

(b) a heavy chain variable region CDR2 comprising SEQ ID NO: 18;

(c) a heavy chain variable region CDR3 comprising SEQ ID NO: 23;

(d) a light chain variable region CDR1 comprising SEQ ID NO: 28;

(e) a light chain variable region CDR2 comprising SEQ ID NO: 33; and

(f) a light chain variable region CDR3 comprising SEQ ID NO: 38.

Another particular combination comprises:

(a) a heavy chain variable region CDR1 comprising SEQ ID NO: 14;

(b) a heavy chain variable region CDR2 comprising SEQ ID NO: 19;

(c) a heavy chain variable region CDR3 comprising SEQ ID NO: 24;

(d) a light chain variable region CDR1 comprising SEQ ID NO: 29;

(e) a light chain variable region CDR2 comprising SEQ ID NO: 34; and

(f) a light chain variable region CDR3 comprising SEQ ID NO: 39.

Another particular combination comprises:

(a) a heavy chain variable region CDR1 comprising SEQ ID NO: 15;

(b) a heavy chain variable region CDR2 comprising SEQ ID NO: 20;

(c) a heavy chain variable region CDR3 comprising SEQ ID NO: 25;

(d) a light chain variable region CDR1 comprising SEQ ID NO: 30;

(e) a light chain variable region CDR2 comprising SEQ ID NO: 35; and

(f) a light chain variable region CDR3 comprising SEQ ID NO: 40.

Other particular antibodies of this disclosure or antigen binding portions thereof comprise:

(a) a heavy chain variable region comprising the amino acid sequence of SEQ ID NO: 1; and

(b) a light chain variable region comprising the amino acid sequence of SEQ ID NO: 6. Another particular combination comprises:

(a) a heavy chain variable region comprising the amino acid sequence of SEQ ID NO: 2; and

(b) a light chain variable region comprising the amino acid sequence of SEQ ID NO: 7.

Another particular combination comprises:

(a) a heavy chain variable region comprising the amino acid sequence of SEQ ID NO: 3; and

(b) a light chain variable region comprising the amino acid sequence of SEQ ID NO: 8.

Another particular combination comprises:

(a) a heavy chain variable region comprising the amino acid sequence of SEQ ID NO: 4; and

(b) a light chain variable region comprising the amino acid sequence of SEQ ID NO: 9. Another particular combination comprises:

(a) a heavy chain variable region comprising the amino acid sequence of SEQ ID NO: 5; and

(b) a light chain variable region comprising the amino acid sequence of SEQ ID NO: 10.

In another aspect of this disclosure, antibody-partner molecule conjugates comprising antibodies or antigen-binding portions thereof, are provided that compete for binding to B7-H4 with any of the aforementioned antibodies.

The antibodies of this disclosure can be, for example, full-length antibodies, for example of an IgG1, IgG2 or IgG4 isotype. Alternatively, the antibodies can be antibody fragments, such as Fab, Fab′ or Fab′2 fragments or single chain antibodies (e.g., scFv).

This disclosure also provides an antibody-partner molecule conjugate comprising an antibody of this disclosure or antigen-binding portion thereof, linked to a therapeutic agent, such as a cytotoxin or a radioactive isotope. In a particularly preferred embodiment, the invention provides an antibody-partner molecule conjugate comprising an antibody of this disclosure, or antigen-binding portion thereof, linked to the compound “Toxin A” (e.g., via a thiol linkage). For example, in various embodiments, the invention provides the following preferred antibody-partner molecule conjugates:

(i) an antibody-partner molecule conjugate comprising an antibody, or antigen-binding portion thereof, comprising:

(a) a heavy chain variable region comprising the amino acid sequence of SEQ ID NO: 1 and a light chain variable region comprising the amino acid sequence of SEQ ID NO: 6;

(b) a heavy chain variable region comprising the amino acid sequence of SEQ ID NO: 2 and a light chain variable region comprising the amino acid sequence of SEQ ID NO: 7;

(c) a heavy chain variable region comprising the amino acid sequence of SEQ ID NO: 3 and a light chain variable region comprising the amino acid sequence of SEQ ID NO: 8;

(d) a heavy chain variable region comprising the amino acid sequence of SEQ ID NO: 4 and a light chain variable region comprising the amino acid sequence of SEQ ID NO: 9; or

(e) a heavy chain variable region comprising the amino acid sequence of SEQ ID NO: 5 and a light chain variable region comprising the amino acid sequence of SEQ ID NO: 10; where the antibody or antigen binding portion thereof is linked to a toxin, such as Toxin A, which is discussed in detail in U.S. Pat. App. No. 60/882,461, which is hereby incorporated by reference in its entirety;

(ii) an antibody-partner molecule conjugate comprising an antibody, or antigen-binding portion thereof, comprising:

(a) a heavy chain variable region CDR1 comprising SEQ ID NO: 11;

(b) a heavy chain variable region CDR2 comprising SEQ ID NO: 16;

(c) a heavy chain variable region CDR3 comprising SEQ ID NO: 21;

(d) a light chain variable region CDR1 comprising SEQ ID NO: 26;

(e) a light chain variable region CDR2 comprising SEQ ID NO: 31; and

(f) a light chain variable region CDR3 comprising SEQ ID NO: 36; or

an antibody, or antigen-binding portion thereof, comprising:

(a) a heavy chain variable region CDR1 comprising SEQ ID NO: 12;

(b) a heavy chain variable region CDR2 comprising SEQ ID NO: 17;

(c) a heavy chain variable region CDR3 comprising SEQ ID NO: 22;

(d) a light chain variable region CDR1 comprising SEQ ID NO: 27;

(e) a light chain variable region CDR2 comprising SEQ ID NO: 32; and

(f) a light chain variable region CDR3 comprising SEQ ID NO: 37; or

an antibody, or antigen-binding portion thereof, comprising:

(a) a heavy chain variable region CDR1 comprising SEQ ID NO: 13;

(b) a heavy chain variable region CDR2 comprising SEQ ID NO: 18;

(c) a heavy chain variable region CDR3 comprising SEQ ID NO: 23;

(d) a light chain variable region CDR1 comprising SEQ ID NO: 28;

(e) a light chain variable region CDR2 comprising SEQ ID NO: 33; and

(f) a light chain variable region CDR3 comprising SEQ ID NO: 38; or

an antibody, or antigen-binding portion thereof, comprising:

(a) a heavy chain variable region CDR1 comprising SEQ ID NO: 14;

(b) a heavy chain variable region CDR2 comprising SEQ ID NO: 19;

(c) a heavy chain variable region CDR3 comprising SEQ ID NO: 24;

(d) a light chain variable region CDR1 comprising SEQ ID NO: 29;

(e) a light chain variable region CDR2 comprising SEQ ID NO: 34; and

(f) a light chain variable region CDR3 comprising SEQ ID NO: 39; or

an antibody, or antigen-binding portion thereof, comprising:

(a) a heavy chain variable region CDR1 comprising SEQ ID NO: 15;

(b) a heavy chain variable region CDR2 comprising SEQ ID NO: 20;

(c) a heavy chain variable region CDR3 comprising SEQ ID NO: 25;

(d) a light chain variable region CDR1 comprising SEQ ID NO: 30;

(e) a light chain variable region CDR2 comprising SEQ ID NO: 35; and

(f) a light chain variable region CDR3 comprising SEQ ID NO: 40; linked to a toxin, such as Toxin A; and

(iii) an antibody-partner molecule conjugate comprising an antibody, or antigen-binding portion thereof, that binds to the same epitope that is recognized by (e.g., cross-competes for binding to human B7-H4 with) an antibody comprising a heavy chain variable region comprising the amino acid sequence of:

(a) a heavy chain variable region comprising the amino acid sequence of SEQ ID NO: 1 and a light chain variable region comprising the amino acid sequence of SEQ ID NO: 6;

(b) a heavy chain variable region comprising the amino acid sequence of SEQ ID NO: 2 and a light chain variable region comprising the amino acid sequence of SEQ ID NO: 7;

(c) a heavy chain variable region comprising the amino acid sequence of SEQ ID NO: 3 and a light chain variable region comprising the amino acid sequence of SEQ ID NO: 8;

(d) a heavy chain variable region comprising the amino acid sequence of SEQ ID NO: 4 and a light chain variable region comprising the amino acid sequence of SEQ ID NO: 9; or

(e) a heavy chain variable region comprising the amino acid sequence of SEQ ID NO: 5 and a light chain variable region comprising the amino acid sequence of SEQ ID NO: 10; where the antibody or antigen binding portion thereof is linked to a toxin, such as Toxin A.

This disclosure also provides a bispecific molecule comprising an antibody or antigen-binding portion thereof, of this disclosure, linked to a second functional moiety having a different binding specificity than said antibody or antigen binding portion thereof.

Compositions comprising an antibody or antigen-binding portion thereof or antibody-partner molecule conjugate or bispecific molecule of this disclosure and a pharmaceutically acceptable carrier are also provided.

Nucleic acid molecules encoding the antibodies or antigen-binding portions thereof, of this disclosure are also encompassed by this disclosure, as well as expression vectors comprising such nucleic acids, host cells comprising such expression vectors and methods for making anti-B7-H4 antibodies using such host cells. Moreover, this disclosure provides a transgenic mouse comprising human immunoglobulin heavy and light chain transgenes, wherein the mouse expresses an antibody of this disclosure, as well as hybridomas prepared from such a mouse, wherein the hybridoma produces the antibody of this disclosure.

The present disclosure also provides isolated anti-B7-H4 antibody-partner molecule conjugates that specifically bind to B7-H4 with high affinity, particularly those comprising human monoclonal antibodies. Certain of such antibody-partner molecule conjugates are capable of being internalized into B7-H4-expressing cells and are capable of mediating antibody dependent cellular cytotoxicity. This disclosure also provides methods for treating cancers, such as breast and ovarian cancers, using an anti-B7-H4 antibody-partner molecule conjugate disclosed herein.

Compositions comprising an antibody, or antigen-binding portion thereof, conjugated to a partner molecule of this disclosure are also provided. Partner molecules that can be advantageously conjugated to an antibody in an antibody partner molecule conjugate as disclosed herein include, but are not limited to, molecules as drugs, toxins, marker molecules (e.g., radioisotopes), proteins and therapeutic agents. Compositions comprising antibody-partner molecule conjugates and pharmaceutically acceptable carriers are also disclosed herein.

In one aspect, such antibody-partner molecule conjugates are conjugated via chemical linkers. In some embodiments, the linker is a peptidyl linker, and is depicted herein as (L⁴)p-F-(L¹)ₘ. Other linkers include hydrazine and disulfide linkers, and is depicted herein as (L⁴)ₚ-H-(L¹)ₘ or (L⁴)ₚ-J-(L¹)ₘ, respectively. In addition to the linkers as being attached to the partner, the present invention also provides cleavable linker arms that are appropriate for attachment to essentially any molecular species.

In yet another aspect, this disclosure provides a method of treating or preventing a disease characterized by growth of tumor cells expressing B7-H4, comprising administering to a subject an antibody-partner molecule conjugate comprising an anti-B7-H4 human antibody of the present disclosure in an amount effective to treat or prevent the disease. The disease can be a cancer, such as a breast cell carcinoma cancer, or an ovarian cancer.

In yet another aspect, this disclosure provides a method of treating an autoimmune disorder, comprising administering to a subject an antibody-partner molecule conjugate comprising an anti-B7-H4 human antibody of the present disclosure in an amount effective to treat the autoimmune disorder.

Other features and advantages of the instant disclosure will be apparent from the following detailed description and examples which should not be construed as limiting. The contents of all references, Genbank entries, patents and published patent applications cited throughout this application are expressly incorporated herein by reference.

### BRIEF DESCRIPTION OF THE DRAWINGS

FIG. 1A shows the nucleotide sequence (SEQ ID NO: 41) and amino acid sequence (SEQ ID NO: 1) of the heavy chain variable region of the 1G11 human monoclonal antibody. The CDR1 (SEQ ID NO: 11), CDR2 (SEQ ID NO: 16) and CDR3 (SEQ ID NO: 21) regions are delineated and the V and J germline derivations are indicated.

FIG. 1B shows the nucleotide sequence (SEQ ID NO: 46) and amino acid sequence (SEQ ID NO: 6) of the light chain variable region of the 1G11 human monoclonal antibody. The CDR1 (SEQ ID NO: 26), CDR2 (SEQ ID NO: 31) and CDR3 (SEQ ID NO: 36) regions are delineated and the V and J germline derivations are indicated.

FIG. 2A shows the nucleotide sequence (SEQ ID NO: 42) and amino acid sequence (SEQ ID NO: 2) of the heavy chain variable region of the 2A7 human monoclonal antibody. The CDR1 (SEQ ID NO: 12), CDR2 (SEQ ID NO: 17) and CDR3 (SEQ ID NO: 22) regions are delineated and the V, D, and J germline derivations are indicated.

FIG. 2B shows the nucleotide sequence (SEQ ID NO: 47) and amino acid sequence (SEQ ID NO: 7) of the light chain variable region of the 2A7 human monoclonal antibody. The CDR1 (SEQ ID NO: 27), CDR2 (SEQ ID NO: 32) and CDR3 (SEQ ID NO: 37) regions are delineated and the V and J germline derivations are indicated.

FIG. 3A shows the nucleotide sequence (SEQ ID NO: 43) and amino acid sequence (SEQ ID NO: 3) of the heavy chain variable region of the 2F9 human monoclonal antibody. The CDR1 (SEQ ID NO: 13), CDR2 (SEQ ID NO: 18) and CDR3 (SEQ ID NO: 23) regions are delineated and the V, D and J germline derivations are indicated.

FIG. 3B shows the nucleotide sequence (SEQ ID NO: 48) and amino acid sequence (SEQ ID NO: 8) of the light chain variable region of the 2F9 human monoclonal antibody. The CDR1 (SEQ ID NO: 28), CDR2 (SEQ ID NO: 33) and CDR3 (SEQ ID NO: 38) regions are delineated and the V and J germline derivations are indicated.

FIG. 4A shows the nucleotide sequence (SEQ ID NO: 44) and amino acid sequence (SEQ ID NO: 4) of the heavy chain variable region of the 12E1 human monoclonal antibody. The CDR1 (SEQ ID NO: 14), CDR2 (SEQ ID NO: 19) and CDR3 (SEQ ID NO: 24) regions are delineated and the V, D and J germline derivations are indicated.

FIG. 4B shows the nucleotide sequence (SEQ ID NO: 49) and amino acid sequence (SEQ ID NO: 9) of the light chain variable region of the 12E1 human monoclonal antibody. The CDR1 (SEQ ID NO: 29), CDR2 (SEQ ID NO: 34) and CDR3 (SEQ ID NO: 39) regions are delineated and the V and J germline derivations are indicated.

FIG. 5A shows the nucleotide sequence (SEQ ID NO: 45) and amino acid sequence (SEQ ID NO: 5) of the heavy chain variable region of the 13D12 human monoclonal antibody. The CDR1 (SEQ ID NO: 15), CDR2 (SEQ ID NO: 20) and CDR3 (SEQ ID NO: 25) regions are delineated and the V, D and J germline derivations are indicated.

FIG. 5B shows the nucleotide sequence (SEQ ID NO: 50) and amino acid sequence (SEQ ID NO: 10) of the light chain variable region of the 13D12 human monoclonal antibody. The CDR1 (SEQ ID NO: 30), CDR2 (SEQ ID NO: 35) and CDR3 (SEQ ID NO: 40) regions are delineated and the V and J germline derivations are indicated.

FIG. 6 shows the alignment of the amino acid sequence of the heavy chain variable region of 1G11 and 13D12 with the human germline V_{H} 4-34 amino acid sequence (SEQ ID NO: 51).

FIG. 7 shows the alignment of the amino acid sequence of the heavy chain variable region of 2A7 and 2F9 with the human germline V_{H} 3-53 amino acid sequence (SEQ ID NO: 52).

FIG. 8 shows the alignment of the amino acid sequence of the heavy chain variable region of 12E1 with the combined human germline V_{H} 3-9/D3-10/JH6b amino acid sequence (SEQ ID NO:53).

FIG. 9 shows the alignment of the amino acid sequence of the light chain variable region of 1G11, 2A7, 2F9 and 13D12 with the human germline V_{K} A27 amino acid sequence (SEQ ID NO:54).

FIG. 10 shows the alignment of the amino acid sequence of the light chain variable region of 12E1 with the combined human germline V_{K} L6/JK1 amino acid sequence (SEQ ID NO:55).

FIGS. 11A and 11B show the results of ELISA experiments demonstrating that human monoclonal antibodies against human O8E specifically bind to O8E. FIG. 11A shows results from an ELISA plate coated with human anti-O8E antibodies followed by the addition of purified O8E protein and detection with rabbit anti-O8E antisera. FIG. 11B shows results from an ELISA plate coated with anti-mouse Fc followed by monoclonal anti-C9 (0.6 μg/ml), then titrated with Penta-O8E protein as indicated and followed by human anti-O8E antibodies at 1 μg/ml.

FIG. 12 shows the results of flow cytometry experiments demonstrating that the anti-O8E human monoclonal antibody 2A7 binds to O8E transfected CHO cells.

FIG. 13 shows the results of flow cytometry experiments demonstrating expression of O8E in SKBR3 breast carcinoma cells as well as O8E transfected SKOV3 and HEK cells.

FIG. 14 shows the results of Hum-Zap internalization experiments demonstrating that human monoclonal antibodies against human O8E can internalize into O8E⁺ CHO cells.

FIG. 15 shows the results of Hum-Zap internalization experiments demonstrating that human monoclonal antibodies against human O8E can internalize into O8E⁺ SKBR3 cells.

FIG. 16 shows the results of epitope mapping studies with various human anti-O8E monoclonal antibodies including 1G11, 2A7, 2F9 and 13D12.

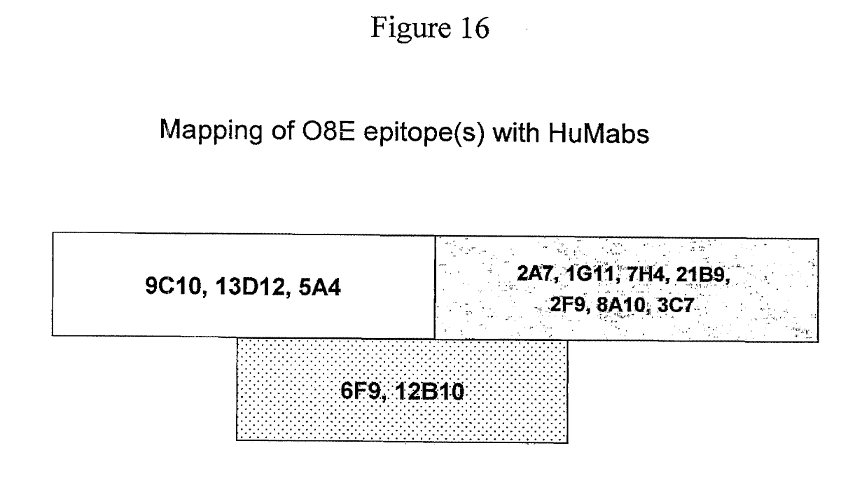

FIG. 17 shows the results of antibody dependent cellular cytotoxicity (ADCC) assays demonstrating that human monoclonal anti-O8E antibodies kill human breast cancer cell line SKBR3 in an ADCC dependent manner.

FIG. 18 shows the results of antibody dependent cellular cytotoxicity (ADCC) assays demonstrating that human monoclonal anti-O8E antibodies kill O8E transfected SKOV3 cells in an ADCC dependent manner.

FIG. 19 shows the results of antibody dependent cellular cytotoxicity (ADCC) assays demonstrating that human monoclonal anti-O8E antibodies kill human breast cancer cell line SKBR3 in a concentration and ADCC dependent manner.

FIG. 20 shows the results of in vivo studies on SCID mice showing tumor growth inhibition of HEK-B7H4 tumors by anti-O8E antibodies.

FIG. 21 presents a graph showing the results of an in vivo HEK293-B7H4 xenograft mouse model, presenting median tumor volume in mice treated with vehicle alone, naked antibody, or antibody-partner molecule conjugates at various concentrations.

FIG. 22 presents a graph showing the results of an in vivo HEK293-B7H4 xenograft mouse model, presenting median body weight change in mice treated with vehicle alone, naked antibody, or antibody-partner molecule conjugates at various concentrations.

### DETAILED DESCRIPTION

The present disclosure relates to antibody-partner molecule conjugates comprising monoclonal antibodies, particularly human sequence monoclonal antibodies, which bind specifically to B7-H4 (a/k/a O8E, B7S1 and B7x) with high affinity. In certain embodiments, the antibodies of this disclosure are derived from particular heavy and light chain germline sequences and/or comprise particular structural features such as CDR regions comprising particular amino acid sequences. This disclosure provides isolated antibodies, methods of making such antibodies, antibody-partner molecule conjugates and bispecific molecules comprising such antibodies and pharmaceutical compositions containing the antibodies, antibody-partner molecule conjugates, or bispecific molecules of this disclosure. This disclosure also relates to methods of using the antibody-partner molecule conjugates, such as to detect B7-H4, as well as to treat diseases associated with expression of B7-H4, such as cancer. Accordingly, this disclosure also provides methods of using the anti-B7-H4 antibody-partner molecule conjugates of this disclosure to treat various cancers, for example, in the treatment of breast cell carcinomas, metastatic breast cancers, ovarian cell carcinomas, metastatic ovarian cancers and renal cell carcinomas.

In order that the present disclosure may be more readily understood, certain terms are first defined. Additional definitions are set forth throughout the detailed description.

The terms “B7-H4,” “O8E,” “B7x” and “B7S1” are used herein interchangeably and include variants, isoforms, homologs, orthologs and paralogs of human B7-H4. For example, antibodies specific for B7-H4 may, in certain cases, cross-react with B7-H4 from species other than human. In other embodiments, the antibodies specific for human B7-H4 may be completely specific for human B7-H4 and may not exhibit species or other types of cross-reactivity. The term “human B7-H4” refers to human sequence B7-H4, such as the complete amino acid sequence of human B7-H4 having Genbank accession number NP 078902 (SEQ ID NO:56). B7-H4 is also known in the art as, for example, BL-CAM, B3, Leu-14 and Lyb-8. The human B7-H4 sequence may differ from human B7-H4 of SEQ ID NO:56 by having, for example, conserved mutations or mutations in non-conserved regions and the B7-H4 has substantially the same biological function as the human B7-H4 of SEQ ID NO:56. For example, a biological function of human B7-H4 is having an epitope in the extracellular domain of B7-H4 that is specifically bound by an antibody of the instant disclosure or a biological function of human B7-H4 includes, for example, inhibition of T-cell proliferation, inhibition of cytokine production, inhibition of cell cycle production, or binding to T cell receptors.

A particular human B7-H4 sequence will generally be at least 90% identical in amino acids sequence to human B7-H4 of SEQ ID NO:56 and contains amino acid residues that identify the amino acid sequence as being human when compared to B7-H4 amino acid sequences of other species (e.g., murine). In certain cases, a human B7-H4 may be at least 95%, or even at least 96%, 97%, 98%, or 99% identical in amino acid sequence to B7-H4 of SEQ ID NO:56. In certain embodiments, a human B7-H4 sequence will display no more than 10 amino acid differences from the B7-H4 of SEQ ID NO:56. In certain embodiments, the human B7-H4 may display no more than 5, or even no more than 4, 3, 2, or 1 amino acid difference from the B7-H4 of SEQ ID NO:56. Percent identity can be determined as described herein.

The term “immune response” refers to the action of for example, lymphocytes, antigen presenting cells, phagocytic cells, granulocytes and soluble macromolecules produced by the above cells or the liver (including antibodies, cytokines and complement) that results in selective damage to, destruction of or elimination from the human body of invading pathogens, cells or tissues infected with pathogens, cancerous cells or, in cases of autoimmunity or pathological inflammation, normal human cells or tissues.

A “signal transduction pathway” refers to the biochemical relationship between a variety of signal transduction molecules that play a role in the transmission of a signal from one portion of a cell to another portion of a cell. As used herein, the phrase “cell surface receptor” includes, for example, molecules and complexes of molecules capable of receiving a signal and the transmission of such a signal across the plasma membrane of a cell. An example of a “cell surface receptor” of the present disclosure is the B7-H4 receptor.

The term “antibody” as referred to herein includes whole antibodies and any antigen binding fragment (i.e., “antigen-binding portion”) or single chains thereof. An “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. The heavy chain constant region is comprised of three domains, C_{H}1, C_{H}2 and C_{H}3. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen-binding portion” of an antibody (or “antibody portion”), as used herein, refers to one or more fragments of an antibody that retain the ability to specifically bind to an antigen (e.g., B7-H4). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and C_{H}1 domains; (ii) a F(ab′)₂ fragment, a bivalent fragment comprising two Fab fragments linked by a disulfide bridge at the hinge region; (iii) a Fab′ fragment, which is essentially an Fab with part of the hinge region (see, FUNDAMENTAL IMMUNOLOGY (Paul ed., 3^{rd} ed. 1993); (iv) a Fd fragment consisting of the V_{H} and C_{H}1 domains; (v) a Fv fragment consisting of the V_{L} and V_{H} domains of a single arm of an antibody, (vi) a dAb fragment (Ward et al., (1989) Nature 341:544-546), which consists of a V_{II} domain; (vii) an isolated complementarity determining region (CDR); and (viii) a nanobody, a heavy chain variable region containing a single variable domain and two constant domains. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

An “isolated antibody”, as used herein, is intended to refer to an antibody that is substantially free of other antibodies having different antigenic specificities (e.g., an isolated antibody that specifically binds B7-H4 is substantially free of antibodies that specifically bind antigens other than B7-H4). An isolated antibody that specifically binds B7-H4 may, however, have cross-reactivity to other antigens, such as B7-H4 molecules from other species. Moreover, an isolated antibody may be substantially free of other cellular material and/or chemicals.

The terms “monoclonal antibody” or “monoclonal antibody composition” as used herein refer to a preparation of antibody molecules of single molecular composition. A monoclonal antibody composition displays a single binding specificity and affinity for a particular epitope. The term “human antibody” or “human sequence antibody”, as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies may include later modifications, including natural or synthetic modifications. The human antibodies of this disclosure may include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences.

The term “human monoclonal antibody”, which may include the term “sequence” after “human”, refers to antibodies displaying a single binding specificity which have variable regions in which both the framework and CDR regions are derived from human germline immunoglobulin sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The term “recombinant human antibody,” as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

As used herein, “isotype” refers to the antibody class (e.g., IgM or IgG1) that is encoded by the heavy chain constant region genes. The phrases “an antibody recognizing an antigen” and “an antibody specific for an antigen” are used interchangeably herein with the term “an antibody which binds specifically to an antigen.”

The term “human antibody derivatives” refers to any modified form of the human antibody, e.g., a conjugate of the antibody and another agent or antibody. The term “humanized antibody” is intended to refer to antibodies in which CDR sequences derived from the germline of another mammalian species, such as a mouse, have been grafted onto human framework sequences. Additional framework region modifications may be made within the human framework sequences.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

The term “antibody mimetic” is intended to refer to molecules capable of mimicking an antibody's ability to bind an antigen, but which are not limited to native antibody structures. Examples of such antibody mimetics include, but are not limited to, Affibodies, DARPins, Anticalins, Avimers, and Versabodies, all of which employ binding structures that, while they mimic traditional antibody binding, are generated from and function via distinct mechanisms.

As used herein, the term “partner molecule” refers to the entity which is conjugated to an antibody in an antibody-partner molecule conjugate. Examples of partner molecules include drugs, toxins, marker molecules (including, but not limited to peptide and small molecule markers such as fluorochrome markers, as well as single atom markers such as radioisotopes), proteins and therapeutic agents.

As used herein, an antibody that “specifically binds to human B7-H4” is intended to refer to an antibody that binds to human B7-H4 with a KD of 1×10⁻⁷ or less, more typically 5×10⁻⁸ M or less, more typically 3×10⁻⁸ M or less, more typically 1×10⁻⁸ M or less, even More typically 5×10⁻⁹M or less.

The term “does not substantially bind” to a protein or cells, as used herein, means does not bind or does not bind with a high affinity to the protein or cells, i.e. binds to the protein or cells with a K_{D} of 1×10⁻⁶ M or more, more preferably 1×10⁻⁵ M or more, more preferably 1×10⁻⁴ M or more, more preferably 1×10⁻³ M or more, even more preferably 1×10⁻² M or more.

The term “K_{assoc}” or “Kₐ,” as used herein, is intended to refer to the association rate of a particular antibody-antigen interaction, whereas the term “K_{dis}” or “K_{d},” as used herein, is intended to refer to the dissociation rate of a particular antibody-antigen interaction. The term “K_{D},” as used herein, is intended to refer to the dissociation constant, which is obtained from the ratio of K_{d} to Kₐ (i.e., K_{d}/Kₐ) and is expressed as a molar concentration (M). K_{D} values for antibodies can be determined using methods well established in the art. A preferred method for determining the K_{D} of an antibody is by using surface plasmon resonance, preferably using a biosensor system such as a Biacore® system.

As used herein, the term “high affinity” for an IgG antibody refers to an antibody having a K_{D} of 1×10⁻⁷ M or less, more preferably 5×10⁻⁸ M or less, even more preferably 1×10⁻⁸ M or less, even more preferably 5×10⁻⁹ M or less and even more preferably 1×10⁻⁹ M or less for a target antigen. However, “high affinity” binding can vary for other antibody isotypes. For example, “high affinity” binding for an IgM isotype refers to an antibody having a K_{D} of 10⁻⁶ M or less, more preferably 10⁻⁷ M or less, even more preferably 10⁻⁸ M or less.

As used herein, the term “subject” includes any human or nonhuman animal. The term “nonhuman animal” includes all vertebrates, e.g., mammals and non-mammals, such as nonhuman primates, sheep, dogs, cats, horses, cows, chickens, amphibians, reptiles, etc.

The symbol “-”, whether utilized as a bond or displayed perpendicular to a bond, indicates the point at which the displayed moiety is attached to the remainder of the molecule, solid support, etc.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, pentadienyl), ethynyl, 1- and 3-propynyl, 3-butynyl, and the higher homologs and isomers.

The term “alkyl,” unless otherwise noted, is also meant to include those derivatives of alkyl defined in more detail below, such as “heteroalkyl.” Alkyl groups, which are limited to hydrocarbon groups are termed “homoalkyl”.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si, and S, and wherein the nitrogen, carbon and sulfur atoms may optionally be oxidized and the nitrogen heteroatom may optionally be quaternized. The heteroatom(s) O, N, S, and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). The terms “heteroalkyl” and “heteroalkylene” encompass poly(ethylene glycol) and its derivatives (see, for example, Shearwater Polymers Catalog, 2001). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

The term “lower” in combination with the terms “alkyl” or “heteroalkyl” refers to a moiety having from 1 to 6 carbon atoms.

The terms “alkoxy,” “alkylamino,” “alkylsulfonyl,” and “alkylthio” (or thioalkoxy) are used in their conventional sense, and refer to those alkyl groups attached to the remainder of the molecule via an oxygen atom, an amino group, an SO₂ group or a sulfur atom, respectively. The term “arylsulfonyl” refers to an aryl group attached to the remainder of the molecule via an SO₂ group, and the term “sulfhydryl” refers to an SH group.

In general, an “acyl substituent” is also selected from the group set forth above. As used herein, the term “acyl substituent” refers to groups attached to, and fulfilling the valence of a carbonyl carbon that is either directly or indirectly attached to the polycyclic nucleus of the compounds of the present invention.

The terms “cycloalkyl” and “heterocycloalkyl”, by themselves or in combination with other terms, represent, unless otherwise stated, cyclic versions of substituted or unsubstituted “alkyl” and substituted or unsubstituted “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 4-morpholinyl, 3-morpholinyl, tetrahydrofuran-2-tetrahydrofuran-3-yl, tetrahydrothien-2-yl, tetrahydrothien-3-yl, 1-piperazinyl, 2-piperazinyl, and the like. The heteroatoms and carbon atoms of the cyclic structures are optionally oxidized.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “aryl” means, unless otherwise stated, a substituted or unsubstituted polyunsaturated, aromatic, hydrocarbon substituent which can be a single ring or multiple rings (preferably from 1 to 3 rings) which are fused together or linked covalently. The term “heteroaryl” refers to aryl groups (or rings) that contain from one to four heteroatoms selected from N, O, and S, wherein the nitrogen, carbon and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5 thiazolyl, 2-furyl, 3-furyl, 2-thienyl, 3-thienyl, 2-pyridyl, 3-pyridyl, 4-pyridyl, 5-benzothiazolyl, purinyl, 2-benzimidazolyl, 5-indolyl, 1-isoquinolyl, 5 isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. “Aryl” and “heteroaryl” also encompass ring systems in which one or more non-aromatic ring systems are fused, or otherwise bound, to an aryl or heteroaryl system.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) include both substituted and unsubstituted forms of the indicated radical. Preferred substituents for each type of radical are provided below.

Substituents for the alkyl, and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generally referred to as “alkyl substituents” and “heteroalkyl substituents,” respectively, and they can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to (2 m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5, 6, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, the aryl substituents and heteroaryl substituents are generally referred to as “aryl substituents” and “heteroaryl substituents,” respectively and are varied and selected from, for example: halogen, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl. When a compound of the invention includes more than one R group, for example, each of the R groups is independently selected as are each R′, R″, R′″ and R″″ groups when more than one of these groups is present.

Two of the aryl substituents on adjacent atoms of the aryl or heteroaryl ring may optionally be replaced with a substituent of the formula -T-C(O)—(CRR′)_{q}—U—, wherein T and U are independently —NR—, —O—, —CRR′— or a single bond, and q is an integer of from 0 to 3.

Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—(CR″R′″)_{d}—, where s and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₆) alkyl.

As used herein, the term “diphosphate” includes but is not limited to an ester of phosphoric acid containing two phosphate groups. The term “triphosphate” includes but is not limited to an ester of phosphoric acid containing three phosphate groups. For example, particular drugs having a diphosphate or a triphosphate include:

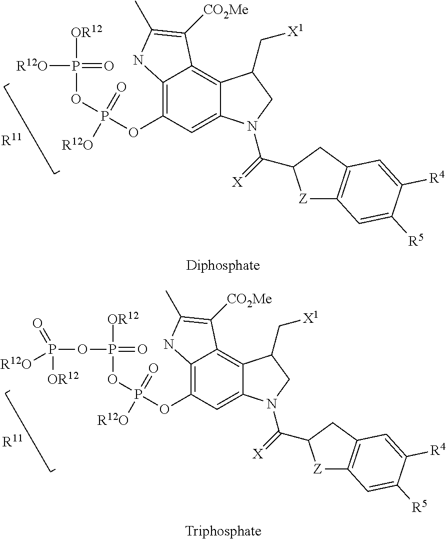

As used herein, the term “heteroatom” includes oxygen (O), nitrogen (N), sulfur (S) and silicon (Si).

The symbol “R” is a general abbreviation that represents a substituent group that is selected from substituted or unsubstituted alkyl, substituted or unsubstituted heteroalkyl, substituted or unsubstituted aryl, substituted or unsubstituted heteroaryl, and substituted or unsubstituted heterocyclyl groups.

Various aspects of the invention are described in further detail in the following subsections

### Anti-B7-H4 Antibodies Having Particular Functional Properties

The antibodies of this disclosure are characterized by particular functional features or properties of the antibodies. For example, the antibodies specifically bind to human B7-H4, such as human B7-H4 expressed on the cell surface. Preferably, an antibody of this disclosure binds to human B7-H4 with high affinity, for example with a K_{D} of 1×10⁻⁷ M or less, more preferably with a K_{D} of 5×10⁻⁸ M or less and even more preferably with a K_{D} of 1×10⁻⁸ M or less. An anti-B7-H4 antibody of this disclosure binds to human B7-H4 and preferably exhibits one or more of the following properties:

(a) binds to human B7-H4 with an affinity of 1×10⁻⁸M or less;

(b) is internalized by B7-H4-expressing cells;

(c) exhibits antibody dependent cellular cytotoxicity (ADCC) against B7-H4 expressing cells; and

(d) inhibits growth of B7-H4-expressing cells in vivo when conjugated to a cytotoxin.

In a preferred embodiment, the antibody exhibits at least two of properties (a), (b), (c), and (d). In a more preferred embodiment, the antibody exhibits at least three of properties (a), (b), (c), and (d). In an even more preferred embodiment, the antibody exhibits all four of properties (a), (b), (c), and (d). In another preferred embodiment, the antibody binds to B7-H4 with an affinity of 5×10⁻⁹ M or less. In yet another preferred embodiment, the antibody inhibits growth of B7-H4-expressing tumor cells in vivo when the antibody is conjugated to a cytotoxin.

Preferably, an antibody of this disclosure binds to a B7-H4 protein with a K_{D} of 5×10⁻⁸ M or less, binds to a B7-H4 protein with a K_{D} of 3×10⁻⁸ M or less, binds to a B7-H4 protein with a K_{D} of 1×10⁻⁸ M or less, binds to a B7-H4 protein with a K_{D} of 7×10⁻⁹ M or less, binds to a B7-H4 protein with a K_{D} of 6×10⁻⁹ M or less or binds to a B7-H4 protein with a K_{D} of 5×10⁻⁹ M or less. The binding affinity of the antibody for B7-H4 can be evaluated, for example, by standard BIACORE analysis.

Standard assays to evaluate the binding ability of the antibodies toward B7-H4 are known in the art, including for example, ELISAs, Western blots, RIAs and flow cytometry analysis. The binding kinetics (e.g., binding affinity) of the antibodies also can be assessed by standard assays known in the art, such as by ELISA, Scatchard and Biacore® system analysis. As another example, the antibodies of the present disclosure may bind to a breast carcinoma tumor cell line, for example, the SKBR3 cell line.

### Monoclonal Antibodies 1G11, 2A7, 2F9, 12E1 and 13D12

Exemplified antibodies of this disclosure include the human monoclonal antibodies 1G11, 2A7, 2F9, 12E1 and 13D12 isolated and structurally characterized as described in PCT Application PCT/US2006/061816, which is hereby incorporated by reference in its entirety. The V_{H} amino acid sequences of 1G11, 2A7, 2F9, 12E1 and 13D12 are shown in SEQ ID NOs: 1, 2, 3, 4 and, 5 respectively. The V_{L} amino acid sequences of 1G11, 2A7, 2F9, 12E1 and 13D12 are shown in SEQ ID NOs: 6, 7, 8, 9 and 10, respectively.

Given that each of these antibodies can bind to B7-H4, the V_{H} and V_{L} sequences can be “mixed and matched” to create other anti-B7-H4 binding molecules of this disclosure. B7-H4 binding of such “mixed and matched” antibodies can be tested using the binding assays described above (e.g., FACS or ELISAs). Preferably, when V_{H} and V_{L} chains are mixed and matched, a V_{H} sequence from a particular V_{H}/V_{L} pairing is replaced with a structurally similar V_{H} sequence. Likewise, typically a V_{L} sequence from a particular V_{H}/V_{L} pairing is replaced with a structurally similar V_{L} sequence. Accordingly, in one aspect, this disclosure provides an isolated monoclonal antibody or antigen binding portion thereof comprising:

(a) a heavy chain variable region comprising an amino acid sequence selected from the group consisting of SEQ ID NOs: 1, 2, 3, 4 and 5; and

(b) a light chain variable region comprising an amino acid sequence selected from the group consisting of SEQ ID NOs: 6, 7, 8, 9 and 10; wherein the antibody specifically binds to B7-H4, preferrably human B7-H4.

Preferred heavy and light chain combinations include:

(a) a heavy chain variable region comprising the amino acid sequence of SEQ ID NO: 1; and

(b) a light chain variable region comprising the amino acid sequence of SEQ ID NO: 6; or

(c) a heavy chain variable region comprising the amino acid sequence of SEQ ID NO: 2; and

(d) a light chain variable region comprising the amino acid sequence of SEQ ID NO: 7; or

(e) a heavy chain variable region comprising the amino acid sequence of SEQ ID NO: 3; and

(f) a light chain variable region comprising the amino acid sequence of SEQ ID NO: 8;

or

(g) a heavy chain variable region comprising the amino acid sequence of SEQ ID NO: 4; and

(h) a light chain variable region comprising the amino acid sequence of SEQ ID NO: 9; or

(i) a heavy chain variable region comprising the amino acid sequence of SEQ ID NO: 5; and

(j) a light chain variable region comprising the amino acid sequence of SEQ ID NO: 10.

In another aspect, this disclosure provides antibodies that comprise the heavy chain and light chain CDR1s, CDR2s and CDR3s of 1G11, 2A7, 2F9, 12E1 and 13D12 or combinations thereof. The amino acid sequences of the V_{H} CDR1s of 1G11, 2A7, 2F9, 12E1 and 13D12 are shown in SEQ ID NOs: 11, 12, 13, 14 and 15, respectively. The amino acid sequences of the V_{H} CDR2s of 1G11, 2A7, 2F9, 12E1 and 13D12 are shown in SEQ ID NOs: 16, 17, 18, 19 and 20, respectively. The amino acid sequences of the V_{H} CDR3s of 1G11, 2A7, 2F9, 12E1 and 13D12 are shown in SEQ ID NOs: 21, 22, 23, 24 and 25, respectively. The amino acid sequences of the V_{K} CDR1s of 1G11, 2A7, 2F9, 12E1 and 13D12 are shown in SEQ ID NOs: 26, 27, 28, 29 and 30, respectively. The amino acid sequences of the V_{K} CDR2s of 1G11, 2A7, 2F9, 12E1 and 13D12 are shown in SEQ ID NOs: 31, 32, 33, 34 and 35, respectively. The amino acid sequences of the V_{K} CDR3s of 1G11, 2A7, 2F9, 12E1 and 13D12 are shown in SEQ ID NOs: 36, 37, 38, 39 and 40, respectively. The CDR regions are delineated using the Kabat system (Kabat, E. A., et al. (1991) Sequences of Proteins of Immunological Interest, Fifth Edition, U.S. Department of Health and Human Services, NIH Publication No. 91-3242).

Given that each of the human antibodies designated 1G11, 2A7, 2F9, 12E1 and 13D12 can bind to B7-H4 and that antigen-binding specificity is provided primarily by the CDR1, CDR2 and CDR3 regions, the V_{H} CDR1, CDR2 and CDR3 sequences and V_{K} CDR1, CDR2 and CDR3 sequences can be “mixed and matched” (i.e. CDRs from different antibodies can be mixed and matched, although each antibody must contain a V_{H} CDR1, CDR2 and CDR3 and a V_{K} CDR1, CDR2 and CDR3) to create other anti-B7-H4 binding molecules of this disclosure. B7-H4 binding of such “mixed and matched” antibodies can be tested using the binding assays described above (e.g., FACS, ELISAs, Biacore® system analysis). Preferably, when V_{H} CDR sequences are mixed and matched, the CDR1, CDR2 and/or CDR3 sequence from a particular V_{H} sequence is replaced with a structurally similar CDR sequence(s). Likewise, when V_{K} CDR sequences are mixed and matched, the CDR1, CDR2 and/or CDR3 sequence from a particular V_{K} sequence typically is replaced with a structurally similar CDR sequence(s). It will be readily apparent to the ordinarily skilled artisan that novel V_{H} and V_{L} sequences can be created by substituting one or more V_{H} and/or V_{L} CDR region sequences with structurally similar sequences from the CDR sequences disclosed herein for monoclonal antibodies 1G11, 2A7, 2F9, 12E1 and 13D12. Accordingly, in another aspect, this disclosure provides an isolated monoclonal antibody or antigen binding portion thereof comprising:

(a) a heavy chain variable region CDR1 comprising an amino acid sequence selected from the group consisting of SEQ ID NOs: 11, 12, 13, 14 and 15;

(b) a heavy chain variable region CDR2 comprising an amino acid sequence selected from the group consisting of SEQ ID NOs: 16, 17, 18, 19 and 20;

(c) a heavy chain variable region CDR3 comprising an amino acid sequence selected from the group consisting of SEQ ID NOs: 21, 22, 23, 24 and 25;

(d) a light chain variable region CDR1 comprising an amino acid sequence selected from the group consisting of SEQ ID NOs: 26, 27, 28, 29 and 30;

(e) a light chain variable region CDR2 comprising an amino acid sequence selected from the group consisting of SEQ ID NOs: 31, 32, 33, 34 and 35; and

(f) a light chain variable region CDR3 comprising an amino acid sequence selected from the group consisting of SEQ ID NOs: 36, 37, 38, 39 and 40; wherein the antibody specifically binds B7-H4, preferably human B7-H4.

In a preferred embodiment, the antibody comprises:

(a) a heavy chain variable region CDR1 comprising SEQ ID NO: 11;

(b) a heavy chain variable region CDR2 comprising SEQ ID NO: 16;

(c) a heavy chain variable region CDR3 comprising SEQ ID NO: 21;

(d) a light chain variable region CDR1 comprising SEQ ID NO: 26;

(e) a light chain variable region CDR2 comprising SEQ ID NO: 31; and

(f) a light chain variable region CDR3 comprising SEQ ID NO: 36.

In another preferred embodiment, the antibody comprises:

(a) a heavy chain variable region CDR1 comprising SEQ ID NO: 12;

(b) a heavy chain variable region CDR2 comprising SEQ ID NO: 17;

(c) a heavy chain variable region CDR3 comprising SEQ ID NO: 22;

(d) a light chain variable region CDR1 comprising SEQ ID NO: 27;

(e) a light chain variable region CDR2 comprising SEQ ID NO: 32; and

(f) a light chain variable region CDR3 comprising SEQ ID NO: 37.

In another preferred embodiment, the antibody comprises:

(a) a heavy chain variable region CDR1 comprising SEQ ID NO: 13;

(b) a heavy chain variable region CDR2 comprising SEQ ID NO: 18;

(c) a heavy chain variable region CDR3 comprising SEQ ID NO: 23;

(d) a light chain variable region CDR1 comprising SEQ ID NO: 28;

(e) a light chain variable region CDR2 comprising SEQ ID NO: 33; and

(f) a light chain variable region CDR3 comprising SEQ ID NO: 38.

In another preferred embodiment, the antibody comprises:

(a) a heavy chain variable region CDR1 comprising SEQ ID NO: 14;

(b) a heavy chain variable region CDR2 comprising SEQ ID NO: 19;

(c) a heavy chain variable region CDR3 comprising SEQ ID NO: 24;

(d) a light chain variable region CDR1 comprising SEQ ID NO: 29;

(e) a light chain variable region CDR2 comprising SEQ ID NO: 34; and

(f) a light chain variable region CDR3 comprising SEQ ID NO: 39.

In another preferred embodiment, the antibody comprises:

(a) a heavy chain variable region CDR1 comprising SEQ ID NO: 15;

(b) a heavy chain variable region CDR2 comprising SEQ ID NO: 20;

(c) a heavy chain variable region CDR3 comprising SEQ ID NO: 25;

(d) a light chain variable region CDR1 comprising SEQ ID NO: 30;

(e) a light chain variable region CDR2 comprising SEQ ID NO: 35; and

(f) a light chain variable region CDR3 comprising SEQ ID NO: 40.

It is well known in the art that the CDR3 domain, independently from the CDR1 and/or CDR2 domain(s), alone can determine the binding specificity of an antibody for a cognate antigen and that multiple antibodies can predictably be generated having the same binding specificity based on a common CDR3 sequence. See, for example, Klimka et al., British J of Cancer 83(2):252-260 (2000) (describing the production of a humanized anti-CD30 antibody using only the heavy chain variable domain CDR3 of murine anti-CD30 antibody Ki-4); Beiboer et al., J. Mol. Biol. 296:833-849 (2000) (describing recombinant epithelial glycoprotein-2 (EGP-2) antibodies using only the heavy chain CDR3 sequence of the parental murine MOC-31 anti-EGP-2 antibody); Rader et al., Proc. Natl. Acad. Sci. U.S.A. 95:8910-8915 (1998) (describing a panel of humanized anti-integrin αᵥβ₃ antibodies using a heavy and light chain variable CDR3 domain of a murine anti-integrin αᵥβ₃ antibody LM609 wherein each member antibody comprises a distinct sequence outside the CDR3 domain and capable of binding the same epitope as the parent murine antibody with affinities as high or higher than the parent murine antibody); Barbas et al., J. Am. Chem. Soc. 116:2161-2162 (1994) (disclosing that the CDR3 domain provides the most significant contribution to antigen binding); Barbas et al., Proc. Natl. Acad. Sci. U.S.A. 92:2529-2533 (1995) (describing the grafting of heavy chain CDR3 sequences of three Fabs (SI-1, SI-40, and SI-32) against human placental DNA onto the heavy chain of an anti-tetanus toxoid Fab thereby replacing the existing heavy chain CDR3 and demonstrating that the CDR3 domain alone conferred binding specificity); Ditzel et al., J. Immunol. 157:739-749 (1996) (describing grafting studies wherein transfer of only the heavy chain CDR3 of a parent polyspecific Fab LNA3 to a heavy chain of a monospecific IgG tetanus toxoid-binding Fab p313 antibody was sufficient to retain binding specificity of the parent Fab); Berezov et al., BIAjournal 8: Scientific Review 8 (2001) (describing peptide mimetics based on the CDR3 of an anti-HER2 monoclonal antibody; Igarashi et al., J. Biochem (Tokyo) 117:452-7 (1995) (describing a 12 amino acid synthetic polypeptide corresponding to the CDR3 domain of an anti-phosphatidylserine antibody); Bourgeois et al., J. Virol 72:807-10 (1998) (showing that a single peptide derived form the heavy chain CDR3 domain of an anti-respiratory syncytial virus (RSV) antibody was capable of neutralizing the virus in vitro); Levi et al., Proc. Natl. Acad. Sci. U.S.A. 90:4374-8 (1993) (describing a peptide based on the heavy chain CDR3 domain of a murine anti-HIV antibody); Polymenis and Stoller, J. Immunol. 152:5218-5329 (1994) (describing enabling binding of an scFv by grafting the heavy chain CDR3 region of a Z-DNA-binding antibody) and Xu and Davis, Immunity 13:37-45 (2000) (describing that diversity at the heavy chain CDR3 is sufficient to permit otherwise identical IgM molecules to distinguish between a variety of hapten and protein antigens). See also, U.S. Pat. Nos. 6,951,646; 6,914,128; 6,090,382; 6,818,216; 6,156,313; 6,827,925; 5,833,943; 5,762,905 and 5,760,185, describing patented antibodies defined by a single CDR domain. Each of these references is hereby incorporated by reference in its entirety.

Accordingly, within certain aspects, the present disclosure provides monoclonal antibodies comprising one or more heavy and/or light chain CDR3 domain from a non-human antibody, such as a mouse or rat antibody, wherein the monoclonal antibody is capable of specifically binding to B7-H4. Within some embodiments, such inventive antibodies comprising one or more heavy and/or light chain CDR3 domain from a non-human antibody (a) are capable of competing for binding with; (b) retain the functional characteristics; (c) bind to the same

epitope; and/or (d) have a similar binding affinity as the corresponding parental non-human antibody.

Within other aspects, the present disclosure provides monoclonal antibodies comprising one or more heavy and/or light chain CDR3 domain from a first human antibody, such as, for example, a human antibody obtained from a non-human animal, wherein the first human antibody is capable of specifically binding to B7-H4 and wherein the CDR3 domain from the first human antibody replaces a CDR3 domain in a human antibody that is lacking binding specificity for B7-H4 to generate a second human antibody that is capable of specifically binding to B7-H4. Within some embodiments, such inventive antibodies comprising one or more heavy and/or light chain CDR3 domain from the first human antibody (a) are capable of competing for binding with; (b) retain the functional characteristics; (c) bind to the same epitope; and/or (d) have a similar binding affinity as the corresponding parental first human antibody.

### Antibodies Having Particular Germline Sequences

In certain embodiments, an antibody of this disclosure comprises a heavy chain variable region from a particular germline heavy chain immunoglobulin gene and/or a light chain variable region from a particular germline light chain immunoglobulin gene.

For example, in a preferred embodiment, this disclosure provides an isolated monoclonal antibody or an antigen-binding portion thereof, comprising a heavy chain variable region that is the product of or derived from a human V_{H} 4-34 gene, wherein the antibody specifically binds B7-H4. In another preferred embodiment, this disclosure provides an isolated monoclonal antibody or an antigen-binding portion thereof, comprising a heavy chain variable region that is the product of or derived from a human V_{H} 3-53 gene, wherein the antibody specifically binds B7-H4.

In another preferred embodiment, this disclosure provides an isolated monoclonal antibody or an antigen-binding portion thereof, comprising a heavy chain variable region that is the product of or derived from a combined human V_{H} 3-9/D3-10/JH6b gene, wherein the antibody specifically binds B7-H4.

In another preferred embodiment, this disclosure provides an isolated monoclonal antibody or an antigen-binding portion thereof, comprising a light chain variable region that is the product of or derived from a human V_{K} A27 gene, wherein the antibody specifically binds B7-H4.

In another preferred embodiment, this disclosure provides an isolated monoclonal antibody or an antigen-binding portion thereof, comprising a light chain variable region that is the product of or derived from a combined human V_{K} L6/JK1 gene, wherein the antibody specifically binds B7-H4.

In yet another preferred embodiment, this disclosure provides an isolated monoclonal antibody or antigen-binding portion thereof, wherein the antibody:

(a) comprises a heavy chain variable region that is the product of or derived from a human V_{H} 4-34 gene, a human V_{H} 3-53 gene or a combined human V_{H} 3-9/D3-10/JH6b gene (which genes encode the amino acid sequences set forth in SEQ ID NOs: 51, 52 and 53, respectively);

(b) comprises a light chain variable region that is the product of or derived from a human V_{K} A27 gene or a combined human V_{K} L6/JK1 gene (which genes encode the amino acid sequences set forth in SEQ ID NOs: 54 and 55, respectively); and

(c) the antibody specifically binds to B7-H4, typically human B7-H4. Examples of antibodies having V_{H} and V_{K} of V_{H} 4-34 and V_{K} A27, respectively, are 1G11 and 13D1 2. Examples of antibodies having V_{H} and V_{K} of V_{H} 3-53 and V_{K} A27, respectively, are 2A7 and 2F9. An example of an antibody having V_{H} and V_{K} of V_{H} 3-9/D 3-10/JH6b and V_{K} L6/JK1, respectively, is 12E1.

As used herein, a human antibody comprises heavy or light chain variable regions that is “the product of or “derived from” a particular germline sequence if the variable regions of the antibody are obtained from a system that uses human germline immunoglobulin genes.

Such systems include immunizing a transgenic mouse carrying human immunoglobulin genes with the antigen of interest or screening a human immunoglobulin gene library displayed on phage with the antigen of interest. A human antibody that is “the product of or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e. greatest % identity) to the sequence of the human antibody. A human antibody that is “the product of or “derived from” a particular human germline immunoglobulin sequence may contain amino acid differences as compared to the germline sequence, due to, for example, naturally-occurring somatic mutations or intentional introduction of site-directed mutation. However, a selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 95% or even at least 96%, 97%, 98% or 99% identical in amino acid sequence to the amino acid sequence encoded by the germline immunoglobulin gene. Typically, a human antibody derived from a particular human germline sequence will display no more than 10 amino acid differences from the amino acid sequence encoded by the human germline immunoglobulin gene. In certain cases, the human antibody may display no more than 5 or even no more than 4, 3, 2 or 1 amino acid difference from the amino acid sequence encoded by the germline immunoglobulin gene.

### Homologous Antibodies

In yet another embodiment, an antibody of this disclosure comprises heavy and light chain variable regions comprising amino acid sequences that are homologous to the amino acid sequences of the preferred antibodies described herein and wherein the antibodies retain the desired functional properties of the anti-B7-H4 antibodies of this disclosure.

For example, this disclosure provides an antibody-partner molecule conjugate comprising a monoclonal antibody or antigen binding portion thereof, comprising a heavy chain variable region and a light chain variable region, wherein:

(a) the heavy chain variable region comprises an amino acid sequence that is at least 80% homologous to an amino acid sequence selected from the group consisting of SEQ ID NOs: 1, 2, 3, 4; and 5;

(b) the light chain variable region comprises an amino acid sequence that is at least 80% homologous to an amino acid sequence selected from the group consisting of SEQ ID NOs: 6, 7, 8; 9 and 10;

(c) the antibody binds to human B7-H4 with a KD of 1×10⁻⁷ M or less;

(d) the antibody binds to human CHO cells transfected with B7-H4; and/or

(e) the antibody inhibits tumor growth of B7-H4-expressing tumor cells in vivo when conjugated to a cytotoxin.

In various embodiments, the antibody can be, for example, a human antibody, a humanized antibody or a chimeric antibody.

In other embodiments, the V_{H} and/or V_{L} amino acid sequences may be 85%, 90%, 95%, 96%, 97%, 98% or 99% homologous to the sequences set forth above. An antibody having V_{H} and V_{L} regions having high (i.e. 80% or greater) homology to the V_{H} and V_{L} regions of the sequences set forth above, can be obtained by mutagenesis (e.g., site-directed or PCR-mediated mutagenesis) of nucleic acid molecules encoding SEQ ID NOs: 41, 42, 43, 44, 45, 46, 47, 48, 49 and 50, followed by testing of the encoded altered antibody for retained function (i.e., the functions set forth in (c), (d), and (e) above), using the functional assays described herein.

As used herein, the percent homology between two amino acid sequences is equivalent to the percent identity between the two sequences. The percent identity between the two sequences is a function of the number of identical positions shared by the sequences (i.e. % homology=# of identical positions/total # of positions×100), taking into account the number of gaps and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present disclosure can further be used as a “query sequence” to perform a search against public databases to, for example, to identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the antibody molecules of this disclosure. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) are useful. See www.ncbi.nlm.nih.gov.

Antibodies with Conservative Modifications

In certain embodiments, an antibody of this disclosure comprises a heavy chain variable region comprising CDR1, CDR2 and CDR3 sequences and a light chain variable region comprising CDR1, CDR2 and CDR3 sequences, wherein one or more of these CDR sequences comprise specified amino acid sequences based on the preferred antibodies described herein (e.g., 1G11, 2A7, 2F9, 12E1 or 13D12) or conservative modifications thereof and wherein the antibodies retain the desired functional properties of the anti-B7-H4 antibodies of this disclosure.

Accordingly, this disclosure provides an antibody-partner molecule conjugate comprising a monoclonal antibody or antigen binding portion thereof, comprising a heavy chain variable region comprising CDR1, CDR2 and CDR3 sequences and a light chain variable region comprising CDR1, CDR2 and CDR3 sequences, wherein:

(a) the heavy chain variable region CDR3 sequence comprises an amino acid sequence selected from the group consisting of amino acid sequences of SEQ ID NOs: 21, 22, 23, 24 and 25 and conservative modifications thereof;

(b) the light chain variable region CDR3 sequence comprises an amino acid sequence selected from the group consisting of amino acid sequence of SEQ ID NOs: 36, 37, 38, 39 and 40 and conservative modifications thereof;

(c) the antibody binds to human B7-H4 with a KD of 1×10⁻⁷ M or less;

(d) the antibody binds to human CHO cells transfected with B7-H4; and/or

(e) the antibody inhibits tumor growth of B7-H4-expressing tumor cells in vivo when conjugated to a cytotoxin.

In a preferred embodiment, the heavy chain variable region CDR2 sequence comprises an amino acid sequence selected from the group consisting of amino acid sequences of SEQ ID NOs: 16, 17, 18, 19 and 20 and conservative modifications thereof; and the light chain variable region CDR2 sequence comprises an amino acid sequence selected from the group consisting of amino acid sequences of SEQ ID NOs: 31, 32, 33, 34 and 35 and conservative modifications thereof. In another preferred embodiment, the heavy chain variable region CDR1 sequence comprises an amino acid sequence selected from the group consisting of amino acid sequences of SEQ ID NOs: 11, 12, 13, 14 and 15 and conservative modifications thereof and the light chain variable region CDR1 sequence comprises an amino acid sequence selected from the group consisting of amino acid sequences of SEQ ID NOs: 26, 27, 28, 29 and 30 and conservative modifications thereof.

In various embodiments, the antibody can be, for example, human antibodies, humanized antibodies or chimeric antibodies.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of this disclosure by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody of this disclosure can be replaced with other amino acid residues from the same side chain family and the altered antibody can be tested for retained function.

Antibodies that Bind to the Same Epitope as Anti-B7-H4 Antibodies of this Disclosure

In another embodiment, this disclosure provides antibodies that bind to the same epitope on human B7-H4 recognized by any of the B7-H4 monoclonal antibodies of this disclosure (i.e. antibodies that have the ability to cross-compete for binding to B7-H4 with any of the monoclonal antibodies of this disclosure). In preferred embodiments, the reference antibody for cross-competition studies can be the monoclonal antibody 1G11 (having V_{H} and V_{L} sequences as shown in SEQ ID NOs: 1 and 6, respectively) or the monoclonal antibody 2A7 (having V_{H} and V_{L} sequences as shown in SEQ ID NOs: 2 and 7, respectively) or the monoclonal antibody 2F9 (having V_{H} and V_{L} sequences as shown in SEQ ID NOs: 3 and 8, respectively) or the monoclonal antibody 12E1 (having V_{H} and V_{L} sequences as shown in SEQ ID NOs: 4 and 9, respectively) or the monoclonal antibody 13D12 (having V_{H} and V_{L} sequences as shown in SEQ ID NOs: 5 and 10, respectively). Such cross-competing antibodies can be identified based on their ability to cross-compete with 1G11, 2A7, 2F9, 12E1 or 13D1 2 in standard B7-H4 binding assays. For example, BIAcore® system analysis, ELISA assays or flow cytometry may be used to demonstrate cross-competition with the antibodies of the current disclosure. The ability of a test antibody to inhibit the binding of for example, 1G11, 2A7, 2F9, 12E1 or 13D12 to human B7-H4 demonstrates that the test antibody can compete with 1G11, 2A7, 2F9, 12E1 or 13D12 for binding to human B7-H4 and thus binds to the same epitope on human B7-H4 as 1G11, 2A7, 2F9, 12E1 or 13D12. In a preferred embodiment, the antibody that binds to the same epitope on human B7-H4 as is recognized by 1G11, 2A7, 2F9, 12E1 or 13D12 is a human monoclonal antibody.

### Engineered and Modified Antibodies

An antibody of this disclosure further can be prepared using an antibody having one or more of the V_{H} and/or V_{L} sequences disclosed herein as starting material to engineer a modified antibody, which modified antibody may have altered properties from the starting antibody. An antibody can be engineered by modifying one or more residues within one or both variable regions (i.e. V_{H} and/or V_{L}), for example within one or more CDR regions and/or within one or more framework regions. Additionally or alternatively, an antibody can be engineered by modifying residues within the constant region(s), for example to alter the effector function(s) of the antibody. One type of variable region engineering that can be performed is CDR grafting.

Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties. (see, e.g., Riechmann, L. et al. (1998) Nature 332:323-327; Jones, P. et al. (1986) Nature 321:522-525; Queen, C. et al. (1989) Proc. Natl. Acad. See. U.S.A. 86:10029-10033; U.S. Pat. No. 5,225,539 to Winter, and U.S. Pat. Nos. 5,530,101; 5,585,089; 5,693,762 and 6,180,370 to Queen et al.)

Accordingly, another embodiment of this disclosure pertains to an isolated monoclonal antibody or antigen binding portion thereof, comprising a heavy chain variable region comprising CDR1, CDR2 and CDR3 sequences comprising an amino acid sequence selected from the group consisting of SEQ ID NOs: 11, 12, 13, 14 and 15; SEQ ID NOs: 16, 17, 18, 19 and 20; and SEQ ID NOs: 21, 22, 23, 24 and 25; respectively and a light chain variable region comprising CDR1, CDR2 and CDR3 sequences comprising an amino acid sequence selected from the group consisting of SEQ ID NOs: 26, 27, 28, 29 and 30; SEQ ID NOs: 31, 32, 33, 34 and 35; and SEQ ID NOs: 36, 37, 38, 39 and 40; respectively. Thus, such antibodies contain the V_{H} and V_{I}, CDR sequences of monoclonal antibodies 1G11, 2A7, 2F9, 12E1 or 13D12 yet may contain different framework sequences from these antibodies.

Such framework sequences can be obtained from public DNA databases or published references that include germline antibody gene sequences. For example, germline DNA sequences for human heavy and light chain variable region genes can be found in the “VBase” human germline sequence database (available on the Internet at www.mrc-cpe.cam.ac.uk/vbase), as well as in Kabat, E. A., et al. (1991) Sequences of Proteins of Immunological Interest, Fifth Edition, U.S. Department of Health and Human Services, NIH Publication No. 91-3242; Tomlinson, I. M., et al. (1992) “The Repertoire of Human Germline V_{H} Sequences Reveals about Fifty Groups of V_{H} Segments with Different Hypervariable Loops” J. Mol. Biol. 227:776-798; and Cox, J. P. L. et al. (1994) “A Directory of Human Germ-line V_{H} Segments Reveals a Strong Bias in their Usage” Eur. J. Immunol. 24:827-836; the contents of each of which are expressly incorporated herein by reference. As another example, the germline DNA sequences for human heavy and light chain variable region genes can be found in the Genbank database. For example, the following heavy chain germline sequences found in the HCo7 HuMAb mouse are available in the accompanying Genbank accession numbers: 1-69 (NG_{—}0010109, NT_{—}024637 and BC070333), 3-33 (NG_{—}0010109 and NT_{—}024637) and 3-7 (NG_{—}0010109 and NT_{—}024637). As another example, the following heavy chain germline sequences found in the HCo12 HuMAb mouse are available in the accompanying Genbank accession numbers: 1-69 (NG_{—}0010109, NT_{—}024637 and BC070333), 5-51 (NG_{—}0010109 and NT_{—}024637), 4-34 (NG_{—}0010109 and NT_{—}024637), 3-30.3 (CAJ556644) and (AJ406678). Yet another source of human heavy and light chain germline sequences is the database of human immunoglobulin genes available from MGT (http://imgt.cines.fr).

Antibody protein sequences are compared against a compiled protein sequence database using one of the sequence similarity searching methods called the Gapped BLAST (Altschul et al. (1997) Nucleic Acids Research 25:3389-3402), which is well known to those skilled in the art. BLAST is a heuristic algorithm in that a statistically significant alignment between the antibody sequence and the database sequence is likely to contain high-scoring segment pairs (HSP) of aligned words. Segment pairs whose scores cannot be improved by extension or trimming is called a hit. Briefly, the nucleotide sequences of VBASE origin (http://vbase.mrc-cpe.cam.ac.uk/vbase1/list2.php) are translated and the region between and including FR1 through FR3 framework region is retained. The database sequences have an average length of 98 residues. Duplicate sequences which are exact matches over the entire length of the protein are removed. A BLAST search for proteins using the program blastp with default, standard parameters except the low complexity filter, which is turned off, and the substitution matrix of BLOSUM62, filters for top 5 hits yielding sequence matches. The nucleotide sequences are translated in all six frames and the frame with no stop codons in the matching segment of the database sequence is considered the potential hit. This is in tarn confirmed using the BLAST program tblastx, which translates the antibody sequence in all six frames and compares those translations to the VBASE nucleotide sequences dynamically translated in all six frames. Other human germline sequence databases, such as that available from IMGT (http://imgt.cines.fr), can be searched similarly to VBASE as described above.

The identities are exact amino acid matches between the antibody sequence and the protein database over the entire length of the sequence. The positives (identities+substitution match) are not identical but amino acid substitutions guided by the BLOSUM62 substitution matrix. If the antibody sequence matches two of the database sequences with same identity, the hit with most positives would be decided to be the matching sequence hit.

Preferred framework sequences for use in the antibodies of this disclosure are those that are structurally similar to the framework sequences used by selected antibodies of this disclosure, e.g., similar to the V_{H} 4-34 framework sequences (SEQ ID NO: 51) and/or the V_{H} 3-53 framework sequences (SEQ ID NO: 52) and/or the combined V_{H} 3-9/D3-10/JH6b framework sequences (SEQ ID NO: 53) and/or the V_{K} A27 framework sequences (SEQ ID NO: 54) and/or the combined V_{K} L6/JK1 framework sequences (SEQ ID NO: 55) used by preferred monoclonal antibodies of this disclosure. The V_{H} CDR1, CDR2 and CDR3 sequences and the V_{K} CDR1, CDR2 and CDR3 sequences, can be grafted onto framework regions that have the identical sequence as that found in the germline immunoglobulin gene from which the framework sequence derive or the CDR sequences can be grafted onto framework regions that contain one or more mutations as compared to the germline sequences. For example, it has been found that in certain instances it is beneficial to mutate residues within the framework regions to maintain or enhance the antigen binding ability of the antibody (see e.g., U.S. Pat. Nos. 5,530,101; 5,585,089; 5,693,762 and 6,180,370 to Queen et al).

Another type of variable region modification is to mutate amino acid residues within the V_{H} and/or V_{K} CDR1, CDR2 and/or CDR3 regions to thereby improve one or more binding properties (e.g., affinity) of the antibody of interest. Site-directed mutagenesis or PCR-mediated mutagenesis can be performed to introduce the mutation(s) and the effect on antibody binding or other functional property of interest, can be evaluated in in vitro or in vivo assays as described herein and provided in the Examples. Typically conservative modifications (as discussed above) are introduced. The mutations may be amino acid substitutions, additions or deletions, but are typically substitutions. Moreover, typically no more than one, two, three, four or five residues within a CDR region are altered.

Accordingly, in another embodiment, this disclosure provides antibody-partner molecule conjugate comprising anti-B7-H4 monoclonal antibodies or antigen binding portions thereof, comprising a heavy chain variable region comprising: (a) a V_{H} CDR1 region comprising an amino acid sequence selected from the group consisting of SEQ ID NOs: 11, 12, 13, 14 and 15 or an amino acid sequence having one, two, three, four or five amino acid substitutions, deletions or additions as compared to SEQ ID NOs: 11, 12, 13, 14 and 15; (b) a V_{H} CDR2 region comprising an amino acid sequence selected from the group consisting of SEQ ID NOs: 16, 17, 18, 19 and 20 or an amino acid sequence having one, two, three, four or five amino acid substitutions, deletions or additions as compared to SEQ ID NOs: 16, 17, 18, 19 and 20; (c) a V_{H} CDR3 region comprising an amino acid sequence selected from the group consisting of SEQ ID NOs: 21, 22, 23, 24 and 25 or an amino acid sequence having one, two, three, four or five amino acid substitutions, deletions or additions as compared to SEQ ID NOs: 21, 22, 23, 24 and 25; (d) a V_{K} CDR1 region comprising an amino acid sequence selected from the group consisting of SEQ ID NOs: 26, 27, 28, 29 and 30 or an amino acid sequence having one, two, three, four or five amino acid substitutions, deletions or additions as compared to SEQ ID NOs: 26, 27, 28, 29 and 30; (e) a V_{K} CDR2 region comprising an amino acid sequence selected from the group consisting of SEQ ID NOs: 31, 32, 33, 34 and 35 or an amino acid sequence having one, two, three, four or five amino acid substitutions, deletions or additions as compared to SEQ ID NOs: 31, 32, 33, 34 and 35; and (f) a V_{K} CDR3 region comprising an amino acid sequence selected from the group consisting of SEQ ID NOs: 36, 37, 38, 39 and 40 or an amino acid sequence having one, two, three, four or five amino acid substitutions, deletions or additions as compared to SEQ ID NOs: 36, 37, 38, 39 and 40.

Engineered antibodies of this disclosure include those in which modifications have been made to framework residues within V_{H} and/or V_{K}, e.g. to improve the properties of the antibody. Typically such framework modifications are made to decrease the immunogenicity of the antibody. For example, one approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived.

For example, for 1G11, amino acid residue #71 (within FR3) of V_{H} is an alanine whereas this residue in the corresponding V_{H} 4-34 germline sequence is a valine. To return the framework region sequences to their germline configuration, the somatic mutations can be “backmutated” to the germline sequence by, for example, site-directed mutagenesis or PCR-mediated mutagenesis (e.g., residue #71 of FR3 of the V_{H} of 1G11 can be “backmutated” from alanine to valine). Such “backmutated” antibodies are also intended to be encompassed by this disclosure.

As another example, for 1G11, amino acid residue #81 (within FR3) of V_{H} is an arginine whereas this residue in the corresponding V_{H} 4-34 germline sequence is a lysine. To return the framework region sequences to their germline configuration, for example, residue #81 of FR3 of the V_{H} of 1G11 can be “backmutated” from arginine to lysine. Such “backmutated” antibodies are also intended to be encompassed by this disclosure.

As another example, for 13D1 2, amino acid residue #83 (within FR3) of V_{H} is an asparagine whereas this residue in the corresponding V_{H} 4-34 germline sequence is a serine. To return the framework region sequences to their germline configuration, for example, residue #83 of FR3 of the V_{H} of 13D1 2 can be “backmutated” from asparagine to serine. Such “backmutated” antibodies are also intended to be encompassed by this disclosure.

As another example, for 2A7, amino acid residue #67 (within FR3) of V_{H} is a valine whereas this residue in the corresponding V_{H} 3-53 germline sequence is an phenylalanine.

To return the framework region sequences to their germline configuration, for example, residue #67 of FR3 of the V_{H} of 2A7 can be “backmutated” from valine to phenylalanine. Such “backmutated” antibodies are also intended to be encompassed by this disclosure.

As another example, for 2F9, amino acid residue #28 (within FR1) of V_{H} is a isoleucine whereas this residue in the corresponding V_{H} 3-53 germline sequence is a threonine. To return the framework region sequences to their germline configuration, for example, residue #28 of FR1 of the V_{H} of 2F9 can be “backmutated” from isoleucine to threonine. Such “backmutated” antibodies are also intended to be encompassed by this disclosure.

As another example, for 12E1, amino acid residue #23 (within FR1) of V_{H} is a valine whereas this residue in the corresponding V_{H} 3-9 germline sequence is an alanine. To return the framework region sequences to their germline configuration, for example, residue #23 of FR1 of the V_{H} of 12E1 can be “backmutated” from valine to alanine. Such “backmutated” antibodies are also intended to be encompassed by this disclosure.

As another example, for 1G11, amino acid residue #7 (within FR1) of V_{K} is a phenylalanine whereas this residue in the corresponding V_{K} A27 germline sequence is a serine. To return the framework region sequences to their germline configuration, for example, residue #7 of FR1 of the V_{K} of 1G11 can be “backmutated” from phenylalanine to serine. Such “backmutated” antibodies are also intended to be encompassed by this disclosure.

As another example, for 1G11, amino acid residue #47 (within FR2) of V_{K} is a valine whereas this residue in the corresponding V_{K} A27 germline sequence is a leucine. To return the framework region sequences to their germline configuration, for example, residue #47 of FR2 of the V_{K} of 1G11 can be “backmutated” from valine to leucine. Such “backmutated” antibodies are also intended to be encompassed by this disclosure.

Another type of framework modification involves mutating one or more residues within the framework region or even within one or more CDR regions, to remove T cell epitopes to thereby reduce the potential immunogenicity of the antibody. This approach is also referred to as “deimmunization” and is described in further detail in U.S. Patent Publication No. 20030153043 by Can et al.

In addition or alternative to modifications made within the framework or CDR regions, antibodies of the invention may be engineered to include modifications within the Fc region, typically to alter one or more functional properties of the antibody, such as serum half-life, complement fixation, Fc receptor binding, and/or antigen-dependent cellular cytotoxicity. Furthermore, an antibody of the invention may be chemically modified (e.g., one or more chemical moieties can be attached to the antibody) or be modified to alter its glycosylation, again to alter one or more functional properties of the antibody. Each of these embodiments is described in further detail below. The numbering of residues in the Fc region is that of the EU index of Kabat.

In one embodiment, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In another embodiment, the Fc hinge region of an antibody is mutated to decrease the biological half life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In another embodiment, the antibody is modified to increase its biological half life. Various approaches are possible. For example, one or more of the following mutations can be introduced: T252L, T254S, T256F, as described in U.S. Pat. No. 6,277,375 to Ward. Alternatively, to increase the biological half life, the antibody can be altered within the CH1 or C_{L} region to contain a salvage receptor binding epitope taken from two loops of a CH2 domain of an Fc region of an IgG, as described in U.S. Pat. Nos. 5,869,046 and 6,121,022 by Presta et al.

In yet other embodiments, the Fc region is altered by replacing at least one amino acid residue with a different amino acid residue to alter the effector function(s) of the antibody. For example, one or more amino acids selected from amino acid residues 234, 235, 236, 237, 297, 318, 320 and 322 can be replaced with a different amino acid residue such that the antibody has an altered affinity for an effector ligand but retains the antigen-binding ability of the parent antibody. The effector ligand to which affinity is altered can be, for example, an Fc receptor or the Cl component of complement. This approach is described in further detail in U.S. Pat. Nos. 5,624,821 and 5,648,260, both by Winter et al.

In another example, one or more amino acids selected from amino acid residues 329, 331 and 322 can be replaced with a different amino acid residue such that the antibody has altered C1q binding and/or reduced or abolished complement dependent cytotoxicity (CDC). This approach is described in further detail in U.S. Pat. No. 6,194,551 by Idusogie et al.

In another example, one or more amino acid residues within amino acid positions 231 and 239 are altered to thereby alter the ability of the antibody to fix complement. This approach is described further in PCT Publication WO 94/29351 by Bodmer et al.

In yet another example, the Fc region is modified to increase the ability of the antibody to mediate antibody dependent cellular cytotoxicity (ADCC) and/or to increase the affinity of the antibody for an Fcγ receptor by modifying one or more amino acids at the following positions: 238, 239, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 329, 330, 331, 333, 334, 335, 337, 338, 340, 360, 373, 376, 378, 382, 388, 389, 398, 414, 416, 419, 430, 434, 435, 437, 438 or 439. This approach is described further in PCT Publication WO 00/42072 by Presta. Moreover, the binding sites on human IgG1 for FcγR1, Fc-γRII, Fc-γRIII and FcRn have been mapped and variants with improved binding have been described (see Shields, R. L. et al. (2001) J. Biol. Chem. 276:6591-6604). Specific mutations at positions 256, 290, 298, 333, 334 and 339 were shown to improve binding to Fc-γRIII. Additionally, the following combination mutants were shown to improve FcγRIII binding: T256A/S298A, S298A/E333A, S298A/K224A and S298A/E333A/K334A.

In still another embodiment, the C-terminal end of an antibody of the present invention is modified by the introduction of a cysteine residue as is described in U.S. Provisional Application Ser. No. 60/957,271, which is hereby incorporated by reference in its entirety. Such modifications include, but are not limited to, the replacement of an existing amino acid residue at or near the C-terminus of a full-length heavy chain sequence, as well as the introduction of a cysteine-containing extension to the c-terminus of a full-length heavy chain sequence. In preferred embodiments, the cysteine-containing extension comprises the sequence alanine-alanine-cysteine (from N-terminal to C-terminal).

In preferred embodiments the presence of such C-terminal cysteine modifications provide a location for conjugation of a partner molecule, such as a therapeutic agent or a marker molecule. In particular, the presence of a reactive thiol group, due to the C-terminal cysteine modification, can be used to conjugate a partner molecule employing the disulfide linkers described in detail below. Conjugation of the antibody to a partner molecule in this manner allows for increased control over the specific site of attachment. Furthermore, by introducing the site of attachment at or near the C-terminus, conjugation can be optimized such that it reduces or eliminates interference with the antibody's functional properties, and allows for simplified analysis and quality control of conjugate preparations.

In still another embodiment, the glycosylation of an antibody is modified. For example, an aglycoslated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 to Co et al., Additional approaches for altering glycosylation are described in further detail in U.S. Pat. No. 7,214,775 to Hanai et al., U.S. Pat. No. 6,737,056 to Presta, U.S. Pub No. 20070020260 to Presta, PCT Publication No. WO/2007/084926 to Dickey et al., PCT Publication No. WO/2006/089294 to Zhu et al., and PCT Publication No. WO/2007/055916 to Ravetch et al., each of which is hereby incorporated by reference in its entirety.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, the cell lines Ms704, Ms705, and Ms709 lack the fucosyltransferase gene, FUT8 (alpha (1,6) fucosyltransferase), such that antibodies expressed in the Ms704, Ms705, and Ms709 cell lines lack fucose on their carbohydrates. The Ms704, Ms705, and Ms709 FUT8^{-/-} cell lines were created by the targeted disruption of the FUT8 gene in CHO/DG44 cells using two replacement vectors (see U.S. Patent Publication No. 20040110704 by Yamane et al. and Yamane-Ohnuki et al. (2004) Biotechnol Bioeng 87:614-22). As another example, EP 1,176,195 by Hanai et al. describes a cell line with a functionally disrupted FUT8 gene, which encodes a fucosyl transferase, such that antibodies expressed in such a cell line exhibit hypofucosylation by reducing or eliminating the alpha 1,6 bond-related enzyme. Hanai et al. also describe cell lines which have a low enzyme activity for adding fucose to the N-acetylglucosamine that binds to the Fc region of the antibody or does not have the enzyme activity, for example the rat myeloma cell line YB2/0 (ATCC CRL 1662). PCT Publication WO 03/035835 by Presta describes a variant CHO cell line, Lec13 cells, with reduced ability to attach fucose to Asn(297)-linked carbohydrates, also resulting in hypofucosylation of antibodies expressed in that host cell (see also Shields, R. L. et al. (2002)J. Biol. Chem. 277:26733-26740). PCT Publication WO 99/54342 by Umana et al. describes cell lines engineered to express glycoprotein-modifying glycosyl transferases (e.g., beta(1,4)-N-acetylglucosaminyltransferase III (GnTIII)) such that antibodies expressed in the engineered cell lines exhibit increased bisecting GlcNac structures which results in increased ADCC activity of the antibodies (see also Umana et al. (1999) Nat. Biotech. 17:176-180). Alternatively, the fucose residues of the antibody may be cleaved off using a fucosidase enzyme. For example, the fucosidase alpha-L-fucosidase removes fucosyl residues from antibodies (Tarentino, A. L. et al. (1975) Biochem. 14:5516-23).

Additionally or alternatively, an antibody can be made that has an altered type of glycosylation, wherein that alteration relates to the level of sialyation of the antibody. Such alterations are described in PCT Publication No. WO/2007/084926 to Dickey et al, and PCT Publication No. WO/2007/055916 to Ravetch et al., both of which are incorporated by reference in their entirety. For example, one may employ an enzymatic reaction with sialidase, such as, for example, Arthrobacter ureafacens sialidase. The conditions of such a reaction are generally described in the U.S. Pat. No. 5,831,077, which is hereby incorporated by reference in its entirety. Other non-limiting examples of suitable enzymes are neuraminidase and N-Glycosidase F, as described in Schloemer et al., J. Virology, 15(4), 882-893 (1975) and in Leibiger et al., Biochem J., 338, 529-538 (1999), respectively. Desialylated antibodies may be further purified by using affinity chromatography.

Alternatively, one may employ methods to increase the level of sialyation, such as by employing sialytransferase enzymes. Conditions of such a reaction are generally described in Basset et al., Scandinavian Journal of Immunology, 51(3), 307-311 (2000).

Another modification of the antibodies herein that is contemplated by the invention is pegylation. An antibody can be pegylated to, for example, increase the biological (e.g., serum) half life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies of the invention. See for example, EP 0 154 316 by Nishimura et al. and EP 0 401 384 by Ishikawa et al.

### Antibody Fragments and Antibody Mimetics

The conjugates of this invention are not limited traditional antibodies as the antigen binding component and may be practiced through the use of antibody fragments and antibody mimetics. A wide variety of antibody fragment and antibody mimetic technologies have now been developed and are widely known in the art.

Domain Antibodies (dAbs) are the smallest functional binding units of antibodies molecular weight approximately 13 kDa—and correspond to the variable regions of either the heavy (VH) or light (VL) chains of antibodies. Further details on domain antibodies and methods of their production are found in U.S. Pat. Nos. 6,291,158; 6,582,915; 6,593,081; 6,172,197; and 6,696,245; US 2004/0110941; EP 1433846, 0368684 and 0616640; WO 2005/035572, 2004/101790, 2004/081026, 2004/058821, 2004/003019 and 2003/002609, each of which is herein incorporated by reference in its entirety.

Nanobodies are antibody-derived proteins that contain the unique structural and functional properties of naturally-occurring heavy-chain antibodies. These heavy-chain antibodies contain a single variable domain (VHH) and two constant domains (CH2 and CH3). Importantly, the cloned and isolated VHH domain is a stable polypeptide harboring the full antigen-binding capacity of the original heavy-chain antibody. Nanobodies have a high homology with the VH domains of human antibodies and can be further humanized without any loss of activity. Importantly, Nanobodies have a low immunogenic potential.

Nanobodies combine the advantages of conventional antibodies with important features of small molecule drugs. Like conventional antibodies, Nanobodies show high target specificity and affinity and low inherent toxicity. Furthermore, Nanobodies are extremely stable, can be administered by means other than injection (see, e.g., WO 2004/041867) and are easy to manufacture. Other advantages of Nanobodies include recognizing uncommon or hidden epitopes as a result of their small size, binding into cavities or active sites of protein targets with high affinity and selectivity due to their unique 3-dimensional, drug format flexibility, tailoring of half-life and ease and speed of drug discovery.

Nanobodies are encoded by single genes and are efficiently produced in almost all prokaryotic and eukaryotic hosts, e.g., E. coli (see, e.g., U.S. Pat. No. 6,765,087, which is herein incorporated by reference in its entirety), molds (for example Aspergillus or Trichoderma) and yeast (for example Saccharomyces, Kluyveromyces, Hansenula or Pichia) (see, e.g., U.S. Pat. No. 6,838,254, which is herein incorporated by reference in its entirety).

The Nanoclone method (see, e.g., WO 06/079372, which is herein incorporated by reference in its entirety) generates Nanobodies against a desired target, based on automated high-throughout selection of B-cells and could be used in the context of the instant invention.

UniBodies are another antibody fragment technology, based upon the removal of the hinge region of IgG4 antibodies. The deletion of the hinge region results in a molecule that is essentially half the size of a traditional IgG4 antibody and has a univalent binding region rather than a bivalent binding region. Furthermore, because UniBodies are about smaller, they may show better distribution over larger solid tumors with potentially advantageous efficacy. Further details on UniBodies may be obtained by reference to WO 2007/059782, which is incorporated by reference in its entirety.

Affibody molecules are affinity proteins based on a 58-amino acid residue protein domain derived from a three helix bundle IgG-binding domain of staphylococcal protein A. This domain has been used as a scaffold for the construction of combinatorial phagemid libraries, from which Affibody variants targeting the desired molecules can be selected using phage display technology (Nord et al., Nat Biotechnol 1997; 15:772-7; Ronmark et al., Eur J Biochem 2002; 269:2647-55). The simple, robust structure and low molecular weight (6 kDa) of Affibody molecules makes them suitable for a wide variety of applications, such as detection reagents and inhibitors of receptor interactions. Further details on Affibodies are found in U.S. Pat. No. 5,831,012 which is incorporated by reference in its entirety. Labeled Affibodies may also be useful in imaging applications for determining abundance of isoforms.

DARPins (Designed Ankyrin Repeat Proteins) embody DRP (Designed Repeat Protein) antibody mimetic technology that exploits the binding abilities of non-antibody polypeptides. Repeat proteins, such as ankyrin and leucine-rich repeat proteins, are ubiquitous binding molecules that, unlike antibodies, occur intra- and extracellularly. Their unique modular architecture features repeating structural units (repeats) that stack together to form elongated repeat domains displaying variable and modular target-binding surfaces. Based on this modularity, combinatorial libraries of polypeptides with highly diversified binding specificities can be generated. This strategy includes the consensus design of self-compatible repeats displaying variable surface residues and their random assembly into repeat domains. Additional information regarding DARPins and other DRP technologies can be found in US 2004/0132028 and WO 02/20565, both of which are incorporated by reference.

Anticalins are another antibody mimetic technology. In this case the binding specificity is derived from lipocalins, a family of low molecular weight proteins that are naturally and abundantly expressed in human tissues and body fluids. Lipocalins have evolved to perform a range of functions in vivo associated with the physiological transport and storage of chemically sensitive or insoluble compounds. Lipocalins have a robust intrinsic structure comprising a highly conserved β-barrel which supports four loops at one terminus of the protein. These loops form the entrance to a binding pocket and conformational differences in this part of the molecule account for the variation in binding specificity between individual lipocalins.

While the overall structure of hypervariable loops supported by a conserved β-sheet framework is reminiscent of immunoglobulins, lipocalins differ considerably from antibodies in terms of size, being composed of a single polypeptide chain of 160-180 amino acids, which is marginally larger than a single immunoglobulin domain.

Lipocalins can be cloned and their loops subjected to engineering to create Anticalins Libraries of structurally diverse Anticalins have been generated and Anticalin display allows the selection and screening of binding function, followed by the expression and production of soluble protein for further analysis in prokaryotic or eukaryotic systems. Studies have demonstrated that Anticalins can be developed that are specific for virtually any human target protein and binding affinities in the nanomolar or higher range can be obtained. Additional information regarding Anticalins can be found in U.S. Pat. No. 7,250,297 and WO 99/16873, both of which are hereby incorporated by reference in their entirety.

Avimers are another type of antibody mimetic technology useful in the context of the instant invention. Avimers are evolved from a large family of human extracellular receptor domains by in vitro exon shuffling and phage display, generating multidomain proteins with binding and inhibitory properties. Linking multiple independent binding domains has been shown to create avidity and results in improved affinity and specificity compared to conventional single-epitope binding proteins. Other potential advantages include simple and efficient production of multi-target-specific molecules in Escherichia coli, improved thermostability and resistance to proteases. Avimers with sub-nanomolar affinities have been obtained against a variety of targets. Additional information regarding Avimers can be found in US 2006/0286603, 2006/0234299, 2006/0223114, 2006/0177831, 2006/0008844, 2005/0221384, 2005/0164301, 2005/0089932, 2005/0053973, 2005/0048512, 2004/0175756, all of which are hereby incorporated by reference in their entirety.

Versabodies are another antibody mimetic technology that can be used in the context of the instant invention. Versabodies are small proteins of 3-5 kDa with >15% cysteines, which form a high disulfide density scaffold replacing the hydrophobic core that typical proteins have. This replacement results in a protein that is smaller, is more hydrophilic (i.e., less prone to aggregation and non-specific binding), is more resistant to proteases and heat, and has a lower density of T-cell epitopes, because the residues that contribute most to MHC presentation are hydrophobic. these properties are well-known to affect immunogenicity, and together they are expected to cause a large decrease in immunogenicity.

Given the structure of Versabodies, these antibody mimetics offer a versatile format that includes multi-valency, multi-specificity, a diversity of half-life mechanisms, tissue targeting modules and the absence of the antibody Fc region. Furthermore, Versabodies are manufactured in E. coli at high yields, and because of their hydrophilicity and small size, Versabodies are highly soluble and can be formulated to high concentrations. Versabodies are exceptionally heat stable and offer extended shelf-life. Additional information regarding Versabodies can be found in US 2007/0191272, which is hereby incorporated by reference in its entirety.

The above descriptions of antibody fragment and mimetic technologies is not intended to be comprehensive. A variety of additional technologies including alternative polypeptide-based technologies, such as fusions of complementarity determining regions as outlined in Qui et al., Nature Biotechnology, 25(8) 921-929 (2007), as well as nucleic acid-based technologies, such as the RNA aptamer technologies described in U.S. Pat. Nos. 5,789,157; 5,864,026; 5,712,375; 5,763,566; 6,013,443; 6,376,474; 6,613,526; 6,114,120; 6,261,774; and 6,387,620; all of which are hereby incorporated by reference, could be used in the context of the instant invention.

### Antibody Physical Properties

The antibodies of the present disclosure may be further characterized by the various physical properties of the anti-B7-H4 antibodies. Various assays may be used to detect and/or differentiate different classes of antibodies based on these physical properties.

In some embodiments, antibodies of the present disclosure may contain one or more glycosylation sites in either the light or heavy chain variable region. The presence of one or more glycosylation sites in the variable region may result in increased immunogenicity of the antibody or an alteration of the pK of the antibody due to altered antigen binding (Marshall et al (1972) Annu Rev Biochem 41:673-702; Gala F A and Morrison S L (2004) J Immunol 172:5489-94; Wallick et al (1988) J Exp Med 168:1099-109; Spiro R G (2002) Glycobiology 12:43R-56R; Parekh et al (1985) Nature 316:452-7; Mimura et al. (2000) Mol Immunol 37:697-706). Glycosylation has been known to occur at motifs containing an N-X-S/T sequence. Variable region glycosylation may be tested using a Glycoblot assay, which cleaves the antibody to produce a Fab, and then tests for glycosylation using an assay that measures periodate oxidation and Schiff base formation. Alternatively, variable region glycosylation may be tested using Dionex light chromatography (Dionex-LC), which cleaves saccharides from a Fab into monosaccharides and analyzes the individual saccharide content. In some instances, it is preferred to have an anti-B7-H4 antibody that does not contain variable region glycosylation. This can be achieved either by selecting antibodies that do not contain the glycosylation motif in the variable region or by mutating residues within the glycosylation motif using standard techniques well known in the art.

In a preferred embodiment, the antibodies of the present disclosure do not contain asparagine isomerism sites. A deamidation or isoaspartic acid effect may occur on N-G or D-G sequences, respectively. The deamidation or isoaspartic acid effect results in the creation of isoaspartic acid which decreases the stability of an antibody by creating a kinked structure off a side chain carboxy terminus rather than the main chain. The creation of isoaspartic acid can be measured using an iso-quant assay, which uses a reverse-phase HPLC to test for isoaspartic acid.

Each antibody will have a unique isoelectric point (pI), but generally antibodies will fall in the pH range of between 6 and 9.5. The pI for an IgG1 antibody typically falls within the pH range of 7-9.5 and the pI for an IgG4 antibody typically falls within the pH range of 6-8. Antibodies may have a pI that is outside this range. Although the effects are generally unknown, there is speculation that antibodies with a pI outside the normal range may have some unfolding and instability under in vivo conditions. The isoelectric point may be tested using a capillary isoelectric focusing assay, which creates a pH gradient and may utilize laser focusing for increased accuracy (Janini et al (2002) Electrophoresis 23:1605-11; Ma et al. (2001) Chromatographia 53:S75-89; Hunt et al (1998) J Chromatogr A 800:355-67). In some instances, it is preferred to have an anti-B7-H4 antibody that contains a pI value that falls in the normal range. This can be achieved either by selecting antibodies with a pI in the normal range, or by mutating charged surface residues using standard techniques well known in the art.

Each antibody will have a melting temperature that is indicative of thermal stability (Krishnamurthy R and Maiming M C (2002) Curr Pharm Biotechnol 3:361-71). A higher thermal stability indicates greater overall antibody stability in vivo. The melting point of an antibody may be measure using techniques such as differential scanning calorimetry (Chen et al (2003) Pharm Res 20:1952-60; Ghirlando et al (1999) Immunol Lett 68:47-52). T_{M1} indicates the temperature of the initial unfolding of the antibody. T_{M2} indicates the temperature of complete unfolding of the antibody. Generally, it is preferred that the T_{M1} of an antibody of the present disclosure is greater than 60° C., preferably greater than 65° C., even more preferably greater than 70° C. Alternatively, the thermal stability of an antibody may be measure using circular dichroism (Murray et al. (2002) J. Chromatogr Sci 40:343-9).

In a preferred embodiment, antibodies are selected that do not rapidly degrade. Fragmentation of an anti-B7-H4 antibody may be measured using capillary electrophoresis (CE) and MALDI-MS, as is well understood in the art (Alexander A J and Hughes D E (1995) Anal Chem 67:3626-32).

In another preferred embodiment, antibodies are selected that have minimal aggregation effects. Aggregation may lead to triggering of an unwanted immune response and/or altered or unfavorable pharmacokinetic properties. Generally, antibodies are acceptable with aggregation of 25% or less, preferably 20% or less, even more preferably 15% or less, even more preferably 10% or less and even more preferably 5% or less. Aggregation may be measured by several techniques well known in the art, including size-exclusion column (SEC) high performance liquid chromatography (HPLC), and light scattering to identify monomers, dimers, trimers or multimers.

### Methods of Engineering Antibodies

As discussed above, the anti-B7-H4 antibodies having V_{H} and V_{K} sequences disclosed herein can be used to create new anti-B7-H4 antibodies by modifying the V_{H} and/or V_{K} sequences or the constant region(s) attached thereto. Thus, in another aspect of this disclosure, the structural features of an anti-B7-H4 antibody of this disclosure, e.g. 1G11, 2A7, 2F9, 12E1 or 13D1 2, are used to create structurally related anti-B7-H4 antibodies that retain at least one functional property of the antibodies of this disclosure, such as binding to human B7-H4. For example, one or more CDR regions of 1G11, 2A7, 2F9, 12E1 or 13D1 2 or mutations thereof, can be combined recombinantly with known framework regions and/or other CDRs to create additional, recombinantly-engineered, anti-B7-H4 antibodies of this disclosure, as discussed above. Other types of modifications include those described in the previous section. The starting material for the engineering method is one or more of the V_{H} and/or V_{K} sequences provided herein or one or more CDR regions thereof. To create the engineered antibody, it is not necessary to actually prepare (i.e. express as a protein) an antibody having one or more of the V_{H} and/or VR sequences provided herein or one or more CDR regions thereof. Rather, the information contained in the sequence(s) is used as the starting material to create a “second generation” sequence(s) derived from the original sequence(s) and then the “second generation” sequence(s) is prepared and expressed as a protein.

Accordingly, in another embodiment, this disclosure provides a method for preparing an anti-B7-H4 antibody comprising:

(a) providing: (i) a heavy chain variable region antibody sequence comprising a CDR1 sequence selected from the group consisting of SEQ ID NOs: 11, 12, 13, 14 and 15, a CDR2 sequence selected from the group consisting of SEQ ID NOs: 16, 17, 18, 19 and 20 and/or a CDR3 sequence selected from the group consisting of SEQ ID NOs: 21, 22, 23, 24 and 25; and/or (ii) a light chain variable region antibody sequence comprising a CDR1 sequence selected from the group consisting of SEQ ID NOs: 26, 27, 28, 29 and 30, a CDR2 sequence selected from the group consisting of SEQ ID NOs: 31, 32, 33, 34 and 35 and/or a CDR3 sequence selected from the group consisting of SEQ ID NOs: 36, 37, 38, 39 and 40;

(b) altering at least one amino acid residue within the heavy chain variable region antibody sequence and/or the light chain variable region antibody sequence to create at least one altered antibody sequence; and

(c) expressing the altered antibody sequence as a protein.

Standard molecular biology techniques can be used to prepare and express the altered antibody sequence. Typically, the antibody encoded by the altered antibody sequence(s) is one that retains one, some or all of the functional properties of the anti-B7-H4 antibodies described herein, which functional properties include, but are not limited to:

(a) binds to human B7-H4 with a KD of 1×10⁻⁷M or less;

(b) binds to human or CHO cells transfected with B7-H4;

(d) the ability to mediate ADCC against B7-H4-expressing cells; and/or

(e) inhibits growth of B7-H4-expressing cells in vivo when conjugated to a cytotoxin.

The functional properties of the altered antibodies can be assessed using standard assays available in the art and/or described herein, such as those set forth in the Examples (e.g., flow cytometry, binding assays).

In certain embodiments of the methods of engineering antibodies of this disclosure, mutations can be introduced randomly or selectively along all or part of an anti-B7-H4 antibody coding sequence and the resulting modified anti-B7-H4 antibodies can be screened for binding activity and/or other functional properties as described herein. Mutational methods have been described in the art. For example, PCT Publication WO 02/092780 by Short describes methods for creating and screening antibody mutations using saturation mutagenesis, synthetic ligation assembly or a combination thereof. Alternatively, PCT Publication WO 03/074679 by Lazar et al. describes methods of using computational screening methods to optimize physiochemical properties of antibodies.

Nucleic Acid Molecules Encoding Antibodies of this Disclosure

Another aspect of this disclosure pertains to nucleic acid molecules that encode the antibodies of this disclosure. The nucleic acids may be present in whole cells, in a cell lysate or in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art. See, F. Ausubel, et al, ed. (1987) Current Protocols in Molecular Biology, Greene Publishing and Wiley Interscience, New York. A nucleic acid of this disclosure can be, for example, DNA or RNA and may or may not contain intronic sequences. In a preferred embodiment, the nucleic acid is a cDNA molecule.

Nucleic acids of this disclosure can be obtained using standard molecular biology techniques. For antibodies expressed by hybridomas hybridomas prepared from transgenic mice carrying human immunoglobulin genes as described further below), cDNAs encoding the light and heavy chains of the antibody made by the hybridoma can be obtained by standard PCR amplification or cDNA cloning techniques. For antibodies obtained from an immunoglobulin gene library {e.g., using phage display techniques), nucleic acid encoding the antibody can be recovered from the library. Preferred nucleic acids molecules of this disclosure are those encoding the V_{H} and V_{L} sequences of the 1G11, 2A7, 2F9, 12E1 or 13D12 monoclonal antibodies. DNA sequences encoding the V_{H} sequences of 1G11, 2A7, 2F9, 12E1 and 13D12 are shown in SEQ ID NOs: 41, 42, 43, 44 and 45, respectively. DNA sequences encoding the V_{L} sequences of 1G11, 2A7, 2F9, 12E1 and 13D12 are shown in SEQ ID NOs: 46, 47, 48, 49 and 50, respectively. Once DNA fragments encoding V_{H} and V_{L} segments are obtained, these DNA fragments can be further manipulated by standard recombinant DNA techniques, for example to convert the variable region genes to full-length antibody chain genes, to Fab fragment genes or to a scFv gene. In these manipulations, a V_{L}- or V_{K}-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the V_{H} region can be converted to a full-length heavy chain gene by operatively linking the V_{H}-encoding DNA to another DNA molecule encoding heavy chain constant regions (CH15 CH2 and CH3). The sequences of human heavy chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region, but most typically is an IgG1 or IgG4 constant region. For a Fab fragment heavy chain gene, the V_{H}-encoding DNA can be operatively linked to another DNA molecule encoding only the heavy chain CH1 constant region.

The isolated DNA encoding the V_{L} region can be converted to a full-length light chain gene (as well as a Fab light chain gene) by operatively linking the V_{L}-encoding DNA to another DNA molecule encoding the light chain constant region, CL. The sequences of human light chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. In preferred embodiments, the light chain constant region can be a kappa or lambda constant region.

To create a scFv gene, the V_{H}- and V_{L}-encoding DNA fragments are operatively linked to another fragment encoding a flexible linker, e.g., encoding the amino acid sequence (Gly₄-Ser)₃, such that the V_{H} and V_{L} sequences can be expressed as a contiguous single-chain protein, with the V_{L} and V_{H} regions joined by the flexible linker (see e.g., Bird et al. (1988) Science 242:423-426; Huston et al. (1988) Proc. Natl. Acad. Sci. USA 85:5879-5883; McCafferty et al., (1990) Nature 348:552-554).

Production of Monoclonal Antibodies of this Disclosure

Monoclonal antibodies (mAbs) of the present disclosure can be produced by a variety of techniques, including conventional monoclonal antibody methodology e.g., the standard somatic cell hybridization technique of Kohler and Milstein (1975) Nature 256: 495. Although somatic cell hybridization procedures are preferred, in principle, other techniques for producing monoclonal antibody can be employed e.g., viral or oncogenic transformation of B lymphocytes.

The preferred animal system for preparing hybridomas is the murine system. Hybridoma production in the mouse is a very well-established procedure. Immunization protocols and techniques for isolation of immunized splenocytes for fusion are known in the art. Fusion partners (e.g., murine myeloma cells) and fusion procedures are also known.

Chimeric or humanized antibodies of the present disclosure can be prepared based on the sequence of a non-human monoclonal antibody prepared as described above. DNA encoding the heavy and light chain immunoglobulins can be obtained from the non-human hybridoma of interest and engineered to contain non-murine (e.g., human) immunoglobulin sequences using standard molecular biology techniques. For example, to create a chimeric antibody, murine variable regions can be linked to human constant regions using methods known in the art (see e.g., U.S. Pat. No. 4,816,567 to Cabilly et al.). To create a humanized antibody, murine CDR regions can be inserted into a human framework using methods known in the art (see e.g., U.S. Pat. No. 5,225,539 to Winter, and U.S. Pat. Nos. 5,530,101; 5,585,089; 5,693,762 and 6,180,370 to Queen et al.).

In a preferred embodiment, the antibodies of this disclosure are human monoclonal antibodies. Such human monoclonal antibodies directed against human B7-H4 can be generated using transgenic or transchromosomic mice carrying parts of the human immune system rather than the mouse system. These transgenic and transchromosomic mice include mice referred to herein as the HuMAb Mouse® and KM Mouse®, respectively, and are collectively referred to herein as “human Ig mice.”

The HuMAb Mouse® (Medarex®, Inc.) contains human immunoglobulin gene miniloci that encode unrearranged human heavy (μ and γ) and κ light chain immunoglobulin sequences, together with targeted mutations that inactivate the endogenous μ and κ chain loci (see e.g., Lonberg, et al. (1994) Nature 368(6474): 856-859). Accordingly, the mice exhibit reduced expression of mouse IgM or K, and in response to immunization, the introduced human heavy and light chain transgenes undergo class switching and somatic mutation to generate high affinity human IgGκ monoclonal antibodies (Lonberg, N. et al. (1994), supra; reviewed in Lonberg, N. (1994) Handbook of Experimental Pharmacology 113:49-101; Lonberg, N. and Huszar, D. (1995) Intern. Rev. Immunol. 13: 65-93, and Harding, F. and Lonberg, N. (1995) Ann. N.Y. Acad. Sci. 764:536-546). Preparation and use of the HuMAb Mouse®, and the genomic modifications carried by such mice, is further described in Taylor, L. et al. (1992) Nucleic Acids Research 20:6287-6295; Chen, J. et al. (1993) International Immunology 5: 647-656; Tuaillon et al. (1993) Proc. Natl. Acad. Sci. USA 90:3720-3724; Choi et al. (1993) Nature Genetics 4:117-123; Chen, J. et al. (1993) EMBO J. 12: 821-830; Tuaillon et al. (1994) J. Immunol. 152:2912-2920; Taylor, L. et al. (1994) International Immunology 6: 579-591; and Fishwild, D. et al. (1996) Nature Biotechnology 14: 845-851, the contents of all of which are hereby specifically incorporated by reference in their entirety. See further, U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg and Kay; U.S. Pat. No. 5,545,807 to Surani et al.; PCT Publication Nos. WO 92/03918, WO 93/12227, WO 94/25585, WO 97/13852, WO 98/24884 and WO 99/45962, all to Lonberg and Kay; and PCT Publication No. WO 01/14424 to Korman et al. Transgenic mice carrying human lambda light chain genes also can be used, such as those described in PCT Publication No. WO 00/26373 by Bruggemann. For example, a mouse carrying a human lambda light chain transgene can be crossbred with a mouse carrying a human heavy chain transgene (e.g., HCo7), and optionally also carrying a human kappa light chain transgene (e.g., KCo5) to create a mouse carrying both human heavy and light chain transgenes.

In another embodiment, human antibodies of this disclosure can be raised using a mouse that carries human immunoglobulin sequences on transgenes and transchomosomes, such as a mouse that carries a human heavy chain transgene and a human light chain transchromosome. This mouse is referred to herein as a “KM Mouse®,” and is described in detail in PCT Publication WO 02/43478 to Ishida et al.

Still further, alternative transgenic animal systems expressing human immunoglobulin genes are available in the art and can be used to raise anti-B7-H4 antibodies of this disclosure. For example, an alternative transgenic system referred to as the Xenomouse (Abgenix, Inc.) can be used; such mice are described in, for example, U.S. Pat. Nos. 5,939,598; 6,075,181; 6,114,598; 6,150,584 and 6,162,963 to Kucherlapati et cal.

Moreover, alternative transchromosomic animal systems expressing human immunoglobulin genes are available in the art and can be used to raise anti-B7-H4 antibodies of this disclosure. For example, mice carrying both a human heavy chain transchromosome and a human light chain tranchromosome, referred to as “TC mice” can be used; such mice are described in Tomizuka et al. (2000) Proc. Natl. Acad. Sci. USA 97:722-727. Furthermore, cows carrying human heavy and light chain transchromosomes have been described in the art (e.g., Kuroiwa et al. (2002) Nature Biotechnology 20:889-894 and PCT application No. WO 2002/092812) and can be used to raise anti-B7-H4 antibodies of this disclosure.

Human monoclonal antibodies of this disclosure can also be prepared using phage display methods for screening libraries of human immunoglobulin genes. Such phage display methods for isolating human antibodies are established in the art. See for example: U.S. Pat. Nos. 5,223,409; 5,403,484; and 5,571,698 to Ladner et al.; U.S. Pat. Nos. 5,427,908 and 5,580,717 to Dower et al.; U.S. Pat. Nos. 5,969,108 and 6,172,197 to McCafferty et al.; and U.S. Pat. Nos. 5,885,793; 6,521,404; 6,544,731; 6,555,313; 6,582,915 and 6,593,081 to Griffiths et al.

Human monoclonal antibodies of this disclosure can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

In another embodiment, human anti-B7-H4 antibodies are prepared using a combination of human Ig mouse and phage display techniques, as described in U.S. Pat. No. 6,794,132 by Buechler et al. More specifically, the method first involves raising an anti-B7-H4 antibody response in a human Ig mouse (such as a HuMab mouse or KM mouse as described above) by immunizing the mouse with one or more B7-H4 antigens, followed by isolating nucleic acids encoding human antibody chains from lymphatic cells of the mouse and introducing these nucleic acids into a display vector (e.g., phage) to provide a library of display packages. Thus, each library member comprises a nucleic acid encoding a human antibody chain and each antibody chain is displayed from the display package. The library then is screened with B7-H4 protein to isolate library members that specifically bind to B7-H4. Nucleic acid inserts of the selected library members then are isolated and sequenced by standard methods to determine the light and heavy chain variable sequences of the selected B7-H4 binders. The variable regions can be converted to full-length antibody chains by standard recombinant DNA techniques, such as cloning of the variable regions into an expression vector that carries the human heavy and light chain constant regions such that the V_{H} region is operatively linked to the C_{H} region and the V_{L} region is operatively linked to the C_{L} region.

### Immunization of Human Ig Mice

When human Ig mice are used to raise human antibodies of this disclosure, such mice can be in with a purified or enriched preparation of a B7-H4 antigen and/or recombinant B7-H4 protein, or cells expressing a B7-H4 protein, or a B7-H4 fusion protein, as described by Lonberg, N. et al. (1994) Nature 368(6474): 856-859; Fishwild, D. et al. (1996) Nature Biotechnology 14: 845-851; and PCT Publication WO 98/24884 and WO 01/14424. Preferably, the mice will be 6-16 weeks of age upon the first infusion. For example, a purified or recombinant preparation (5-50 μg) of B7-H4 antigen can be used to immunize the human Ig mice intraperitoneally and/or subcutaneously. Most preferably, the immunogen used to raise the antibodies of this disclosure is a B7-H4 fusion protein comprising the extracellular domain of a B7-H4 protein, fused at its N-terminus to a non-B7-H4 polypeptide (e.g., a His tag) (described further in Example 1).

Detailed procedures to generate fully human monoclonal antibodies that bind human B7-H4 are described in Example 1 below. Cumulative experience with various antigens has shown that the transgenic mice respond when initially immunized intraperitoneally (IP) with antigen in complete Freund's adjuvant, followed by every other week IP immunizations (up to a total of 6) with antigen in incomplete Freund's adjuvant. However, adjuvants other than Freund's are also found to be effective (e.g., RIBI adjuvant). In addition, whole cells in the absence of adjuvant are found to be highly immunogenic. The immune response can be monitored over the course of the immunization protocol with plasma samples being obtained by retroorbital bleeds. The plasma can be screened by ELISA (as described below), and mice with sufficient titers of anti-B7-H4 human immunoglobulin can be used for fusions. Mice can be boosted intravenously with antigen, for example 3 days before sacrifice and removal of the spleen. It is expected that 2-3 fusions for each immunization may need to be performed. Between 6 and 24 mice are typically immunized for each antigen. Usually both HCo7 and HCo12 strains are used. In addition, both HCo7 and HCo12 transgene can be bred together into a single mouse having two different human heavy chain transgenes (HCo7/HCo12). Alternatively or additionally, the KM Mouse® strain can be used.

### Generation of Hybridomas Producing Human Monoclonal Antibodies of the Invention

To generate hybridomas producing human monoclonal antibodies of this disclosure, splenocytes and/or lymph node cells from immunized mice can be isolated and fused to an appropriate immortalized cell line, such as a mouse myeloma cell line. The resulting hybridomas can be screened for the production of antigen-specific antibodies. For example, single cell suspensions of splenic lymphocytes from immunized mice can be fused to one-sixth the number of P3×63-Ag8.653 nonsecreting mouse myeloma cells (ATCC, CRL 1580) with 50% PEG. Alternatively, the single cell suspension of splenic lymphocytes from immunized mice can be fused using an electric field based electrofusion method, using a CytoPulse large chamber cell fusion electroporator (CytoPulse Sciences, Inc., Glen Burnie Md.). Cells are plated at approximately 2×10⁵ in flat bottom microtiter plate, followed by a two week incubation in selective medium containing 20% fetal Clone Serum, 18% “653” conditioned media, 5% origen (IGEN), 4 mM L-glutamine, 1 mM sodium pyruvate, 5 mM HEPES, 0.055 mM 2-mercaptoethanol, 50 units/ml penicillin, 50 mg/ml streptomycin, 50 mg/ml gentamycin and 1×HAT (Sigma; the HAT is added 24 hours after the fusion). After approximately two weeks, cells can be cultured in medium in which the HAT is replaced with HT. Individual wells can then be screened by ELISA for human monoclonal IgM and IgG antibodies. Once extensive hybridoma growth occurs, medium can be observed usually after 10-14 days. The antibody secreting hybridomas can be replated, screened again, and if still positive for human IgG, the monoclonal antibodies can be subcloned at least twice by limiting dilution. The stable subclones can then be cultured in vitro to generate small amounts of antibody in tissue culture medium for characterization.

To purify human monoclonal antibodies, selected hybridomas can be grown in two-liter spinner-flasks for monoclonal antibody purification. Supernatants can be filtered and concentrated before affinity chromatography with protein A-sepharose (Pharmacia, Piscataway, N.J.). Eluted IgG can be checked by gel electrophoresis and high performance liquid chromatography to ensure purity. The buffer solution can be exchanged into PBS, and the concentration can be determined by OD280 using 1.43 extinction coefficient. The monoclonal antibodies can be aliquoted and stored at −80° C.

### Generation of Transfectomas Producing Monoclonal Antibodies of the Invention

Antibodies of this disclosure also can be produced in a host cell transfectoma using, for example, a combination of recombinant DNA techniques and gene transfection methods as is well known in the art (e.g., Morrison, S. (1985) Science 229:1202).

For example, to express the antibodies, or antibody fragments thereof, DNAs encoding partial or full-length light and heavy chains, can be obtained by standard molecular biology techniques (e.g., PCR amplification or cDNA cloning using a hybridoma that expresses the antibody of interest) and the DNAs can be inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). The light and heavy chain variable regions of the antibodies described herein can be used to create full-length antibody genes of any antibody isotype by inserting them into expression vectors already encoding heavy chain constant and light chain constant regions of the desired isotype such that the V_{H} segment is operatively linked to the C_{H} segment(s) within the vector and the V_{L} segment is operatively linked to the C_{L} segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of this disclosure carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel (Gene Expression Technology. Methods in Enzymology 185, Academic Press, San Diego, Calif. (1990)). It will be appreciated by those skilled in the art that the design of the expression vector, including the selection of regulatory sequences, may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, (e.g., the adenovirus major late promoter (AdMLP) and polyoma. Alternatively, nonviral regulatory sequences may be used, such as the ubiquitin promoter or β-globin promoter. Still further, regulatory elements composed of sequences from different sources, such as the SRα promoter system, which contains sequences from the SV40 early promoter and the long terminal repeat of human T cell leukemia virus type 1 (Takebe, Y. et al. (1988) Mol. Cell. Biol. 8:466-472).

In addition to the antibody chain genes and regulatory sequences, the recombinant expression vectors of this disclosure may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification) and the neo gene (for G418 selection).

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies of this disclosure in either prokaryotic or eukaryotic host cells, expression of antibodies in eukaryotic cells, and most preferably mammalian host cells, is the most preferred because such eukaryotic cells, and in particular mammalian cells, are more likely than prokaryotic cells to assemble and secrete a properly folded and immunologically active antibody. Prokaryotic expression of antibody genes has been reported to be ineffective for production of high yields of active antibody (Boss, M. A. and Wood, C. R. (1985) Immunology Today 6:12-13).

Preferred mammalian host cells for expressing the recombinant antibodies of this disclosure include Chinese Hamster Ovary (CHO cells) (including dhfr⁻ CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) J. Mol. Biol. 159:601-621), NSO myeloma cells, COS cells and SP2 cells. In particular, for use with NSO myeloma cells, another preferred expression system is the GS gene expression system disclosed in WO 87/04462 (to Wilson), WO 89/01036 (to Bebbington) and EP 338,841 (to Bebbington). When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

### Characterization of Antibody Binding to Antigen

Antibodies of the invention can be tested for binding to human B7-H4 by, for example, standard ELISA. Briefly, microtiter plates are coated with purified and/or recombinant B7-H4 protein (e.g., a B7-H4 fusion protein as described in Example 1) at 1 μg/ml in PBS, and then blocked with 5% bovine serum albumin in PBS. Dilutions of antibody (e.g., dilutions of plasma from B7-H4-immunized mice) are added to each well and incubated for 1-2 hours at 37° C. The plates are washed with PBS/Tween and then incubated with secondary reagent (e.g., for human antibodies, a goat-anti-human IgG Fc-specific polyclonal reagent) conjugated to alkaline phosphatase for 1 hour at 37° C. After washing, the plates are developed with pNPP substrate (1 mg/ml), and analyzed at OD of 405-650. Preferably, mice that develop the highest titers will be used for fusions.

An ELISA assay as described above can also be used to screen for hybridomas that show positive reactivity with a B7-H4 protein. Hybridomas that bind with high avidity and/or affinity to a B7-H4 protein are subcloned and further characterized. One clone from each hybridoma, which retains the reactivity of the parent cells (by ELISA), can be chosen for making a 5-10 vial cell bank stored at −140° C., and for antibody purification.

To purify anti-B7-H4 antibodies, selected hybridomas can be grown in two-liter spinner-flasks for monoclonal antibody purification. Supernatants can be filtered and concentrated before affinity chromatography with protein A-sepharose (Pharmacia, Piscataway, N.J.). Eluted IgG can be checked by gel electrophoresis and high performance liquid chromatography to ensure purity. The buffer solution can be exchanged into PBS, and the concentration can be determined by OD280 using 1.43 extinction coefficient. The monoclonal antibodies can be aliquoted and stored at −80° C.

To determine if the selected anti-B7-H4 monoclonal antibodies bind to unique epitopes, each antibody can be biotinylated using commercially available reagents (Pierce, Rockford, Ill.). Competition studies using unlabeled monoclonal antibodies and biotinylated monoclonal antibodies can be performed using B7-H4 protein coated-ELISA plates as described above. Biotinylated mAb binding can be detected with a strep-avidin-alkaline phosphatase probe.

To determine the isotype of purified antibodies, isotype ELISAs can be performed using reagents specific for antibodies of a particular isotype. For example, to determine the isotype of a human monoclonal antibody, wells of microliter plates can be coated with 1 μg/ml of anti-human immunoglobulin overnight at 4° C. After blocking with 1% BSA, the plates are reacted with 1 μg/ml or less of test monoclonal antibodies or purified isotype controls, at ambient temperature for one to two hours. The wells can then be reacted with either human IgG1 or human IgM-specific alkaline phosphatase-conjugated probes. Plates are developed and analyzed as described above.

Anti-B7-H4 human IgGs can be further tested for reactivity with a B7-H4 antigen by Western blotting. Briefly, a B7-H4 protein can be prepared and subjected to sodium dodecyl sulfate polyacrylamide gel electrophoresis. After electrophoresis, the separated antigens are transferred to nitrocellulose membranes, blocked with 10% fetal calf serum, and probed with the monoclonal antibodies to be tested. Human IgG binding can be detected using anti-human IgG alkaline phosphatase and developed with BCIP/NBT substrate tablets (Sigma Chem. Co., St. Louis, Mo.).

The binding specificity of an antibody of this disclosure may also be determined by monitoring binding of the antibody to cells expressing a B7-H4 protein, for example by flow cytometry. Cells or cell lines that naturally expresses B7-H4 protein, such OVCAR3, NCI-11226, CFPAC-1 or KB cells (described further in Example 3), may be used or a cell line, such as a CHO cell line, may be transfected with an expression vector encoding B7-H4 such that B7-H4 is expressed on the surface of the cells. The transfected protein may comprise a tag, such as a myc-tag or a Ins-tag, preferably at the N-terminus, for detection using an antibody to the tag. Binding of an antibody of this disclosure to a B7-H4 protein may be determined by incubating the transfected cells with the antibody, and detecting bound antibody. Binding of an antibody to the tag on the transfected protein may be used as a positive control.

### Bispecific Molecules

In another aspect, the present disclosure features bispecific molecules comprising an anti-B7-H4 antibody, or a fragment thereof, of this disclosure. An antibody of this disclosure, or antigen-binding portions thereof, can be derivatized or linked to another functional molecule, e.g., another peptide or protein (e.g., another antibody or ligand for a receptor) to generate a bispecific molecule that binds to at least two different binding sites or target molecules. The antibody of this disclosure may in fact be derivatized or linked to more than one other functional molecule to generate multispecific molecules that bind to more than two different binding sites and/or target molecules; such multispecific molecules are also intended to be encompassed by the term “bispecific molecule” as used herein. To create a bispecific molecule of this disclosure, an antibody of this disclosure can be functionally linked (e.g., by chemical coupling, genetic fusion, noncovalent association or otherwise) to one or more other binding molecules, such as another antibody, antibody fragment, peptide or binding mimetic, such that a bispecific molecule results.

Accordingly, the present disclosure includes bispecific molecules comprising at least one first binding specificity for B7-H4 and a second binding specificity for a second target epitope. In a particular embodiment of this disclosure, the second target epitope is an Fc receptor, e.g., human FcγRI (CD64) or a human Fcα receptor (CD89). Therefore, this disclosure includes bispecific molecules capable of binding both to FcγR or FcαR expressing effector cells (e.g., monocytes, macrophages or polymorphonuclear cells (PMNs)), and to target cells expressing B7-H4 protein. These bispecific molecules target B7-H4 expressing cells to effector cell and trigger Fc receptor-mediated effector cell activities, such as phagocytosis of B7-H4 expressing cells, antibody dependent cell-mediated cytotoxicity (ADCC), cytokine release, or generation of superoxide anion.

In an embodiment of this disclosure in which the bispecific molecule is multispecific, the molecule can further include a third binding specificity, in addition to an anti-Fc binding specificity and an anti-B7-H4 binding specificity. In one embodiment, the third binding specificity is an anti-enhancement factor (EF) portion, e.g., a molecule which binds to a surface protein involved in cytotoxic activity and thereby increases the immune response against the target cell. The “anti-enhancement factor portion” can be an antibody, functional antibody fragment or a ligand that binds to a given molecule, e.g., an antigen or a receptor, and thereby results in an enhancement of the effect of the binding determinants for the Fc receptor or target cell antigen. The “anti-enhancement factor portion” can bind an Fc receptor or a target cell antigen. Alternatively, the anti-enhancement factor portion can bind to an entity that is different from the entity to which the first and second binding specificities bind. For example, the anti-enhancement factor portion can bind a cytotoxic T-cell (e.g. via CD2, CD3, CD8, CD28, CD4, CD40, ICAM-1 or other immune cell that results in an increased immune response against the target cell).

In one embodiment, the bispecific molecules of this disclosure comprise as a binding specificity at least one antibody, or an antibody fragment thereof; including, e.g., an Fab, Fab′, F(ab′)₂, Fv, Fd, dAb or a single chain Fv. The antibody may also be a light chain or heavy chain dimer, or any minimal fragment thereof such as a Fv or a single chain construct as described in U.S. Pat. No. 4,946,778 to Ladner et al., the contents of which is expressly incorporated by reference.

In one embodiment, the binding specificity for an Fcγ receptor is provided by a monoclonal antibody, the binding of which is not blocked by human immunoglobulin G (IgG). As used herein, the term “IgG receptor” refers to any of the eight γ-chain genes located on chromosome 1. These genes encode a total of twelve transmembrane or soluble receptor isoforms which are grouped into three Fcγ receptor classes: FcγRI (CD64), Fcγ RII (CD32), and FcγRIII (CD16). In one preferred embodiment, the Fcγ receptor is a human high affinity FcγRI. The human FcγRI is a 72 kDa molecule, which shows high affinity for monomeric IgG (10⁸-10⁹ M⁻¹).

The production and characterization of certain preferred anti-Fcγ monoclonal antibodies are described in PCT Publication WO 88/00052 and in U.S. Pat. No. 4,954,617 to Fanger et al., the teachings of which are fully incorporated by reference herein. These antibodies bind to an epitope of FcγRI, FcγRII or FcγRIII at a site which is distinct from the Fcγ binding site of the receptor and, thus, their binding is not blocked substantially by physiological levels of IgG. Specific anti-FcγRI antibodies useful in this disclosure are mAb 22, mAb 32, mAb 44, mAb 62 and mAb 197. The hybridoma producing mAb 32 is available from the American Type Culture Collection, ATCC Accession No. HB9469. In other embodiments, the anti-Fcγ receptor antibody is a humanized form of monoclonal antibody 22 (H22). The production and characterization of the 1122 antibody is described in Graziano, R. F. et al. (1995) J. Immunol 155 (10): 4996-5002 and PCT Publication WO 94/10332 to Tempest et al. The H22 antibody producing cell line was deposited at the American Type Culture Collection under the designation HA022CL1 and has the accession no. CRL 11177.

In still other preferred embodiments, the binding specificity for an Fc receptor is provided by an antibody that binds to a human IgA receptor, e.g., an Fc-alpha receptor (Fcα RI (CD89)), the binding of which is preferably not blocked by human immunoglobulin A (IgA). The term “IgA receptor” is intended to include the gene product of one α-gene (Fcα RI) located on chromosome 19. This gene is known to encode several alternatively spliced transmembrane isoforms of 55 to 110 kDa. FcαRI (CD89) is constitutively expressed on monocytes/macrophages, eosinophilic and neutrophilic granulocytes, but not on non-effector cell populations. FcαRI has medium affinity (≈5×10⁷ M⁻¹) for both IgA1 and IgA2, which is increased upon exposure to cytokines such as G-CSF or GM-CSF (Morton, H. C. et al. (1996) Critical Reviews in Immunology 16:423-440). Four FcαRI-specific monoclonal antibodies, identified as A3, A59, A62 and A77, which bind FcαRI outside the IgA ligand binding domain, have been described (Monteiro, R. C. et al. (1992) J. Immunol. 148:1764).

FcαRI and FcγRI are preferred trigger receptors for use in the bispecific molecules of this disclosure because they are (1) expressed primarily on immune effector cells, e.g., monocytes, PMNs, macrophages and dendritic cells; (2) expressed at high levels (e.g., 5,000-100,000 per cell); (3) mediators of cytotoxic activities (e.g., ADCC, phagocytosis); and (4) mediate enhanced antigen presentation of antigens, including self-antigens, targeted to them.

While human monoclonal antibodies are preferred, other antibodies which can be employed in the bispecific molecules of this disclosure are murine, chimeric and humanized monoclonal antibodies.

The bispecific molecules of the present disclosure can be prepared by conjugating the constituent binding specificities, e.g., the anti-FcR and anti-B7-H4 binding specificities, using methods known in the art. For example, each binding specificity of the bispecific molecule can be generated separately and then conjugated to one another. When the binding specificities are proteins or peptides, a variety of coupling or cross-linking agents can be used for covalent conjugation. Examples of cross-linking agents include protein A, carbodiimide, N-succinimidyl-5-acetyl-thioacetate (SATA), 5,5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl)cyclohaxane-1-carboxylate (sulfo-SMCC) (see e.g., Karpovsky et al. (1984) J. Exp. Med. 160:1686; Liu, M A et al. (1985) Proc. Natl. Acad. Sci. USA 82:8648). Other methods include those described in Paulus (1985) Behring Ins. Mitt. No. 78, 118-132; Brennan et al. (1985) Science 229:81-83, and Glennie et al. (1987) J. Immunol. 139: 2367-2375). Preferred conjugating agents are SATA and sulfo-SMCC, both available from Pierce Chemical Co. (Rockford, Ill.).

When the binding specificities are antibodies, they can be conjugated via sulfhydryl bonding of the C-terminus hinge regions of the two heavy chains. In a particularly preferred embodiment, the hinge region is modified to contain an odd number of sulfhydryl residues, preferably one, prior to conjugation.

Alternatively, both binding specificities can be encoded in the same vector and expressed and assembled in the same host cell. This method is particularly useful where the bispecific molecule is a mAb×mAb, mAb×Fab, Fab×F(ab′)₂ or ligand x Fab fusion protein. A bispecific molecule of this disclosure can be a single chain molecule comprising one single chain antibody and a binding determinant, or a single chain bispecific molecule comprising two binding determinants. Bispecific molecules may comprise at least two single chain molecules. Methods for preparing bispecific molecules are described for example in U.S. Pat. Nos. 5,260,203; 5,455,030; 4,881,175; 5,132,405; 5,091,513; 5,476,786; 5,013,653; 5,258,498; and 5,482,858, all of which are expressly incorporated herein by reference.

Binding of the bispecific molecules to their specific targets can be confirmed by, for example, enzyme-linked immunosorbent assay (ELISA), radioimmunoassay (RIA), FACS analysis, bioassay (e.g., growth inhibition), or Western Blot assay. Each of these assays generally detects the presence of protein-antibody complexes of particular interest by employing a labeled reagent (e.g., an antibody) specific for the complex of interest. For example, the FcR-antibody complexes can be detected using e.g., an enzyme-linked antibody or antibody fragment which recognizes and specifically binds to the antibody-FcR complexes. Alternatively, the complexes can be detected using any of a variety of other immunoassays. For example, the antibody can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a gamma counter or a scintillation counter or by autoradiography.

### Conjugates

In conjugates of this invention, the partner molecule is conjugated to an antibody by a chemical linker (sometimes referred to herein simply as “linker”). The partner molecule can be a therapeutic agent or a marker. The therapeutic agent can be, for example, a cytotoxin, a non-cytotoxic drug (e.g., an immunosuppressant), a radioactive agent, another antibody, or an enzyme. Preferably, the partner molecule is a cytotoxin. The marker can be any label that generates a detectable signal, such as a radiolabel, a fluorescent label, or an enzyme that catalyzes a detectable modification to a substrate. The antibody serves a targeting function: by binding to a target tissue or cell where its antigen is found, the antibody steers the conjugate to the target tissue or cell. There, the linker is cleaved, releasing the partner molecule to perform its desired biological function.

The ratio of partner molecules attached to an antibody can vary, depending on factors such as the amount of partner molecule employed during conjugation reaction and the experimental conditions. Preferably, the ratio of partner molecules to antibody is between 1 and 3, more preferably between 1 and 1.5. Those skilled in the art will appreciate that, while each individual molecule of antibody Z is conjugated to an integer number of partner molecules, a preparation of the conjugate may analyze for a non-integer ratio of partner molecules to antibody, reflecting a statistical average.

### Linkers

In some embodiments, the linker is a peptidyl linker, depicted herein as (L⁴)ₚ-F-(L¹)ₘ. Other linkers include hydrazine and disulfide linkers, depicted herein as (L⁴)ₚ-H-(L¹)ₘ, and (L⁴)ₚ-J-(L¹)ₘ, respectively. F, H, and J are peptidyl, hydrazine, and disulfide moieties, respectively, that are cleavable to release the partner molecule from the antibody, while L¹ and L⁴ are linker groups. F, H, J, L¹, and L⁴ are more fully defined herein below, along with the subscripts p and m. The preparation and use of these and other linkers are described in WO 2005/112919, the disclosure of which is incorporated herein by reference.

The use of peptidyl and other linkers in antibody-partner conjugates is described in US 2006/0004081; 2006/0024317; 2006/0247295; U.S. Pat. No. 6,989,452; U.S. Pat. No. 7,087,600; and U.S. Pat. No. 7,129,261; WO 2007/051081; 2007/038658; 2007/059404; and 2007/089100; all of which are incorporated herein by reference.

Additional linkers are described in U.S. Pat. Nos. 6,214,345; 2003/0096743; and 2003/0130189; de Groot et al., J. Med. Chem. 42, 5277 (1999); de Groot et al. J. Org. Chem. 43, 3093 (2000); de Groot et al., J. Med. Chem. 66, 8815, (2001); WO 02/083180; Carl et al., J. Med. Chem. Lett. 24, 479, (1981); Dubowchik et al., Bioorg & Med. Chem. Lett. 8, 3347 (1998), the disclosures of which are incorporated herein by reference.

In addition to connecting the antibody and the partner molecule, a linker can impart stability to the partner molecule, reduce its in vivo toxicity, or otherwise favorably affect its pharmacokinetics, bioavailability and/or pharmacodynamics. It is generally preferred that the linker is cleaved, releasing the partner molecule, once the conjugate is delivered to its site of action. Also preferably, the linkers are traceless, such that once cleaved, no trace of the linker's presence remains.

In another embodiment, the linkers are characterized by their ability to be cleaved at a site in or near a target cell such as at the site of therapeutic action or marker activity of the partner molecule. Such cleavage can be enzymatic in nature. This feature aids in reducing systemic activation of the partner molecule, reducing toxicity and systemic side effects. Preferred cleavable groups for enzymatic cleavage include peptide bonds, ester linkages, and disulfide linkages, such as the aforementioned F, H, and J moieties. In other embodiments, the linkers are sensitive to pH and are cleaved through changes in pH.

An important aspect is the ability to control the speed with which the linkers cleave. Often a linker that cleaves quickly is desired. In some embodiments, however, a linker that cleaves more slowly may be preferred. For example, in a sustained release formulation or in a formulation with both a quick release and a slow release component, it may be useful to provide a linker which cleaves more slowly. The aforecited WO 2005/112919 discloses hydrazine linkers that can be designed to cleave at a range of speeds, from very fast to very slow.

The linkers can also serve to stabilize the partner molecule against degradation while the conjugate is in circulation, before it reaches the target tissue or cell. This is a significant benefit since it prolongates the circulation half-life of the partner molecule. The linker also serves to attenuate the activity of the partner molecule so that the conjugate is relatively benign while in circulation but the partner molecule has the desired effect—for example is cytotoxic—after activation at the desired site of action. For therapeutic agent conjugates, this feature of the linker serves to improve the therapeutic index of the agent.

In addition to the cleavable peptide, hydrazine, or disulfide groups F, H, or J, respectively, one or more linker groups L¹ are optionally introduced between the partner molecule and F, H, or J, as the case may be. These linker groups L¹ may also be described as spacer groups and contain at least two functional groups. Depending on the value of the subscript m (i.e., the number of L¹ groups present) and the location of a particular group L¹, a chemical functionality of a group L¹ can bond to a chemical functionality of the partner molecule, of F, H or J, as the case may be, or of another linker group L¹ (if more than one L¹ is present). Examples of suitable chemical functionalities for spacer groups L¹ include hydroxy, mercapto, carbonyl, carboxy, amino, ketone, aldehyde, and mercapto groups.

The linkers L¹ can be a substituted or unsubstituted alkyl, substituted or unsubstituted aryl, substituted or unsubstituted heteroaryl or substituted or unsubstituted heteroalkyl group. In one embodiment, the alkyl or aryl groups may comprise between 1 and 20 carbon atoms. They may also comprise a polyethylene glycol moiety.

Exemplary groups L¹ include, for example, 6-aminohexanol, 6-mercaptohexanol, 10-hydroxydecanoic acid, glycine and other amino acids, 1,6-hexanediol, β-alanine, 2-aminoethanol, cysteamine (2-aminoethanethiol), 5-aminopentanoic acid, 6-aminohexanoic acid, 3-maleimidobenzoic acid, phthalide, α-substituted phthalides, the carbonyl group, aminal esters, nucleic acids, peptides and the like.

One function of the groups L¹ is to provide spatial separation between F, H or J, as the case may be, and the partner molecule, lest the latter interfere (e.g., via steric or electronic effects) with cleavage chemistry at F, H, or J. The groups L¹ also can serve to introduce additional molecular mass and chemical functionality into conjugate. Generally, the additional mass and functionality affects the serum half-life and other properties of the conjugate. Thus, through careful selection of spacer groups, conjugates with a range of serum half-lives can be produced. Optionally, one or more linkers L¹ can be a self-immolative group, as described herein below.

The subscript m is an integer selected from 0, 1, 2, 3, 4, 5, and 6. When multiple L¹ groups are present, they can be the same or different.

L⁴ is a linker moiety that provides spatial separation between F, H, or J, as the case may be, and the antibody, lest F, H, or J interfere with the antigen binding by the antibody or the antibody interfere with the cleavage chemistry at F, H, or J. Preferably, L⁴ imparts increased solubility or decreased aggregation properties to conjugates utilizing a linker that contains the moiety or modifies the hydrolysis rate of the conjugate. As in the case of L¹, L⁴ optionally is a self immolative group. In one embodiment, L⁴ is substituted alkyl, unsubstituted alkyl, substituted aryl, unsubstituted aryl, substituted heteroalkyl, or unsubstituted heteroalkyl, any of which may be straight, branched, or cyclic. The substitutions can be, for example, a lower (C₁-C₆) alkyl, alkoxy, alkylthio, alkylamino, or dialkyl-amino. In certain embodiments, L⁴ comprises a non-cyclic moiety. In another embodiment, L⁴ comprises a positively or negatively charged amino acid polymer, such as polylysine or polyarginine L⁴ can comprise a polymer such as a polyethylene glycol moiety. Additionally, L⁴ can comprise, for example, both a polymer component and a small molecule moiety.

In a preferred embodiment, L⁴ comprises a polyethylene glycol (PEG) moiety. The

PEG portion of L⁴ may be between 1 and 50 units long. Preferably, the PEG will have 1-12 repeat units, more preferably 3-12 repeat units, more preferably 2-6 repeat units, or even more preferably 3-5 repeat units and most preferably 4 repeat units. L⁴ may consist solely of the PEG moiety, or it may also contain an additional substituted or unsubstituted alkyl or heteroalkyl. It is useful to combine PEG as part of the L⁴ moiety to enhance the water solubility of the complex. Additionally, the PEG moiety reduces the degree of aggregation that may occur during the conjugation of the drug to the antibody.

The subscript p is 0 or 1; that is, the presence of L⁴ is optional. Where present, L⁴ has at least two functional groups, with one functional group binding to a chemical functionality in F, H, or J, as the case may be, and the other functional group binding to the antibody. Examples of suitable chemical functionalities of groups L⁴ include hydroxy, mercapto, carbonyl, carboxy, amino, ketone, aldehyde, and mercapto groups. As antibodies typically are conjugated via sulfhydryl groups (e.g., from unoxidized cysteine residues, the addition of sulfhydryl-containing extensions to lysine residues with iminothiolane, or the reduction of disulfide bridges), amino groups (e.g., from lysine residues), aldehyde groups (e.g., from oxidation of glycoside side chains), or hydroxyl groups (e.g., from serine residues), preferred chemical functionalities for attachment to the antibody are those reactive with the foregoing groups, examples being maleimide, sulfhydryl, aldehyde, hydrazine, semicarbazide, and carboxyl groups. The combination of a sulfhydryl group on the antibody and a maleimide group on L⁴ is preferred.

In some embodiments, L⁴ comprises

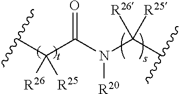

directly attached to the N-terminus of (AA¹)_{c}. R²⁰ is a member selected from H, substituted or unsubstituted alkyl, substituted or unsubstituted heteroalkyl, and acyl. Each R²⁵, R^{25′}, R²⁶, and R^{26′} is independently selected from H, substituted or unsubstituted alkyl, substituted or unsubstituted heteroalkyl, substituted or unsubstituted aryl, substituted or unsubstituted heteroaryl, and substituted or unsubstituted heterocycloalkyl; and s and t are independently integers from 1 to 6. Preferably, R²⁰, R²⁵, R^{25′}, R²⁶ and R^{26′} are hydrophobic. In some embodiments, R²⁰ is H or alkyl (preferably, unsubstituted lower alkyl). In some embodiments, R²⁵, R^{25′}, R²⁶ and R^{26′} are independently H or alkyl (preferably, unsubstituted C¹ to C⁴ alkyl). In some embodiments, R²⁵, R^{25′}, R²⁶ and R^{26′} are all H. In some embodiments, t is 1 and s is 1 or 2.

### Peptide Linkers (F)

As discussed above, the peptidyl linkers of the invention can be represented by the general formula: (L⁴)ₚ-F-(L¹)ₘ, wherein F represents the portion comprising the peptidyl moiety. In one embodiment, the F portion comprises an optional additional self-immolative linker L² and a carbonyl group, corresponding to a conjugate of formula (a):

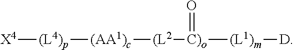

In this embodiment, L¹, L⁴, p, and in are as defined above. X⁴ is an antibody and D is a partner molecule. The subscript o is 0 or 1 and L², if present, represents a self-immolative linker. AA¹ represents one or more natural amino acids, and/or unnatural α-amino acids; c is an integer from 1 and 20. In some embodiments, c is in the range of 2 to 5 or c is 2 or 3.

In formula (a), AA¹ is linked, at its amino terminus, either directly to L⁴ or, when L⁴ is absent, directly to X⁴. In some embodiments, when L⁴ is present, L⁴ does not comprise a carboxylic acyl group directly attached to the N-terminus of (AA¹)_{c}.

In another embodiment, the F portion comprises an amino group and an optional spacer group L³ and L¹ is absent (i.e., m is 0), corresponding to a conjugate of formula (b):

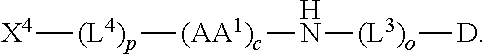

In this embodiment, X⁴, D, L⁴, AA¹, c, and p are as defined above. The subscript o is 0 or 1. L³, if present, is a spacer group comprising a primary or secondary amine or a carboxyl functional group, and either the amine of L³ forms an amide bond with a pendant carboxyl functional group of D or the carboxyl of L³ forms an amide bond with a pendant amine functional group of D.

### Self-Immolative Linkers

A self-immolative linker is a bifunctional chemical moiety which is capable of covalently linking together two spaced chemical moieties into a normally stable tripartate molecule, releasing one of said spaced chemical moieties from the tripartate molecule by means of enzymatic cleavage; and following said enzymatic cleavage, spontaneously cleaving from the remainder of the molecule to release the other of said spaced chemical moieties. In accordance with the present invention, the self-immolative spacer is covalently linked at one of its ends to the peptide moiety and covalently linked at its other end to the chemically reactive site of the drug moiety whose derivatization inhibits pharmacological activity, so as to space and covalently link together the peptide moiety and the drug moiety into a tripartate molecule which is stable and pharmacologically inactive in the absence of the target enzyme, but which is enzymatically cleavable by such target enzyme at the bond covalently linking the spacer moiety and the peptide moiety to thereby effect release of the peptide moiety from the tripartate molecule. Such enzymatic cleavage, in turn, will activate the self-immolating character of the spacer moiety and initiate spontaneous cleavage of the bond covalently linking the spacer moiety to the drug moiety, to thereby effect release of the drug in pharmacologically active form. See, for example, Carl et al., J. Med. Chem., 24 (3), 479-480 (1981); Carl et al., WO 81/01145 (1981); Toki et al., J. Org. Chem. 67, 1866-1872 (2002); Boyd et al., WO 2005/112919; and Boyd et al., WO 2007/038658, the disclosures of which are incorporated herein by reference.

One particularly preferred self-immolative spacer may be represented by the formula (c):

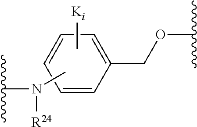

The aromatic ring of the aminobenzyl group may be substituted with one or more “K” groups. A “K” group is a substituent on the aromatic ring that replaces a hydrogen otherwise attached to one of the four non-substituted carbons that are part of the ring structure. The “K” group may be a single atom, such as a halogen, or may be a multi-atom group, such as alkyl, heteroalkyl, amino, intro, hydroxy, alkoxy, haloalkyl, and cyano. Each K is independently selected from the group consisting of substituted alkyl, unsubstituted alkyl, substituted heteroalkyl, unsubstituted heteroalkyl, substituted aryl, unsubstituted aryl, substituted heteroaryl, unsubstituted heteroaryl, substituted heterocycloalkyl, unsubstituted heterocycloalkyl, halogen, NO₂, NR²¹R²², NR²¹COR²², OCONR²¹R²², OCOR²¹, and OR²¹, wherein R²¹ and R²² are independently selected from the group consisting of H, substituted alkyl, unsubstituted alkyl, substituted heteroalkyl, unsubstituted heteroalkyl, substituted aryl, unsubstituted aryl, substituted heteroaryl, unsubstituted heteroaryl, substituted heterocycloalkyl and unsubstituted heterocycloalkyl. Exemplary K substituents include, but are not limited to, F, Cl, Br, I, NO₂, OH, OCH₃, NHCOCH₃, N(CH₃)₂, NHCOCF₃ and methyl. For “Kᵢ”, i is an integer of 0, 1, 2, 3, or 4. In one preferred embodiment, i is 0.

The ether oxygen atom of the above structure is connected to a carbonyl group (not shown). The line from the NR²⁴ functionality into the aromatic ring indicates that the amine functionality may be bonded to any of the five carbons that both form the ring and are not substituted by the —CH₂—O— group. Preferably, the NR²⁴ functionality of X is covalently bound to the aromatic ring at the para position relative to the —CH₂—O— group. R²⁴ is a member selected from the group consisting of H, substituted alkyl, unsubstituted alkyl, substituted heteroalkyl, and unsubstituted heteroalkyl. In a specific embodiment, R²⁴ is hydrogen.

In one embodiment, the invention provides a peptide linker of formula (a) above, wherein F comprises the structure:

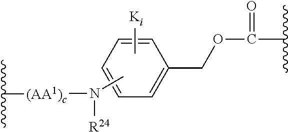

where R²⁴, AA¹, K, i, and c are as defined above.

In another embodiment, the peptide linker of formula (a) above comprises a —F-(L¹)ₘ- that comprises the structure:

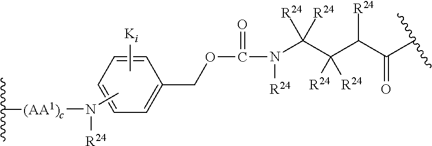

where R²⁴, AA¹, K, i, and c are as defined above.

In some embodiments, a self-immolative spacer L¹ or L² includes

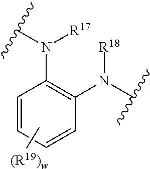

where each R¹⁷, R¹⁸, and R¹⁹ is independently selected from H, substituted or unsubstituted alkyl, substituted or unsubstituted heteroalkyl and substituted or unsubstituted aryl, and w is an integer from 0 to 4. In some embodiments, R¹⁷ and R¹⁸ are independently H or alkyl (preferably, unsubstituted C₁-C₄ alkyl). Preferably, R¹⁷ and R¹⁸ are C₁₋₄ alkyl, such as methyl or ethyl. In some embodiments, w is 0. It has been found experimentally that this particular self-immolative spacer cyclizes relatively quickly.

In some embodiments, L¹ or L² includes

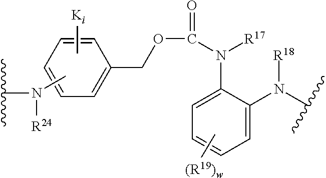

where R¹⁷, R¹⁸, R¹⁹, R²⁴, and K are as defined above.

### Spacer Groups

The spacer group L³ is characterized by comprises a primary or secondary amine or a carboxyl functional group, and either the amine of L³ forms an amide bond with a pendant carboxyl functional group of D or the carboxyl of L³ forms an amide bond with a pendant amine functional group of D. L³ can be selected from the group consisting of substituted or unsubstituted alkyl, substituted or unsubstituted heteroalkyl, substituted or unsubstituted aryl, substituted or unsubstituted heteroaryl, or substituted or unsubstituted heterocycloalkyl. In a preferred embodiment, L³ comprises an aromatic group. More preferably, L³ comprises a benzoic acid group, an aniline group or indole group. Non-limiting examples of structures that can serve as an -L³-NH— spacer include the following structures:

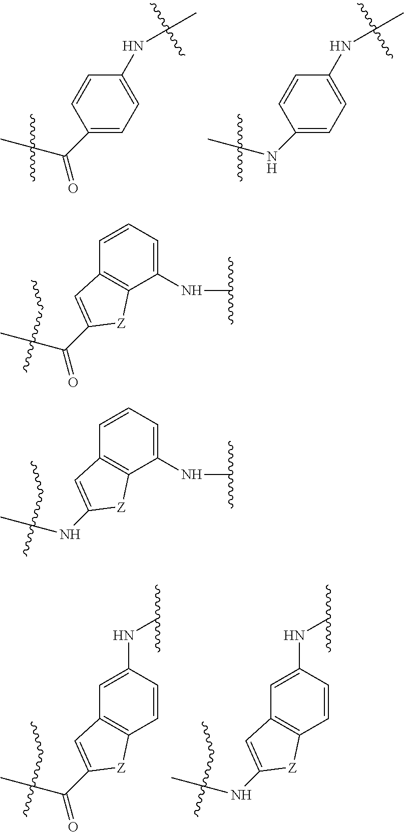

where Z is a member selected from O, S and NR²³, and where R²³ is a member selected from H, substituted or unsubstituted alkyl, substituted or unsubstituted heteroalkyl, and acyl.

Upon cleavage of the linker of the invention containing L³, the L³ moiety remains attached to the drug, D. Accordingly, the L³ moiety is chosen such that its attachment to D does not significantly alter the activity of D. In another embodiment, a portion of the drug D itself functions as the L³ spacer. For example, in one embodiment, the drug, D, is a duocarmycin derivative in which a portion of the drug functions as the L³ spacer. Non-limiting examples of such embodiments include those in which NH₂-(L³)-D has a structure selected from the group consisting of:

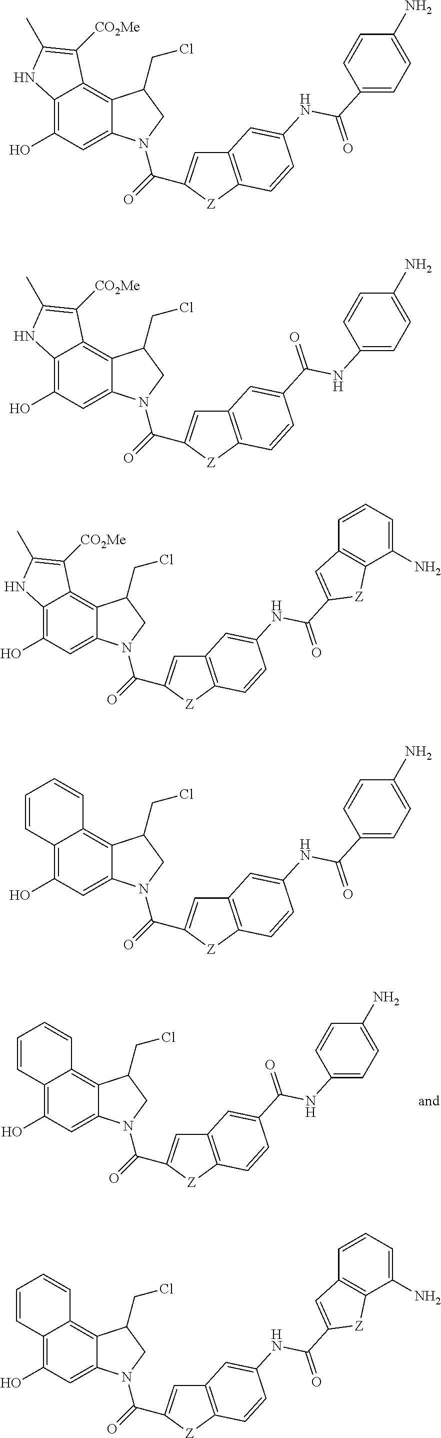

where Z is O, S or NR²³, where R²³ is H, substituted or unsubstituted alkyl, substituted or unsubstituted heteroalkyl, or acyl; and the NH₂ group on each structure reacts with (AA¹)_{c} to form -(AA¹)_{c}-NH—.

### Peptide Sequence (AA¹)_{c}

The group AA¹ represents a single amino acid or a plurality of amino acids joined together by amide bonds. The amino acids may be natural amino acids and/or unnatural α-amino acids. They may be in the L or the D configuration. In one embodiment, at least three different amino acids are used. In another embodiment, only two amino acids are used.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, citrulline, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium Such analogs have modified R groups (e.g., norleucine) or modified peptide backbones, but retain the same basic chemical structure as a naturally occurring amino acid. One amino acid that may be used in particular is citrulline, which is a precursor to arginine and is involved in the formation of urea in the liver. Amino acid mimetics refers to chemical compounds that have a structure that is different from the general chemical structure of an amino acid, but functions in a manner similar to a naturally occurring amino acid. The term “unnatural amino acid” is intended to represent the “D” stereochemical form of the twenty naturally occurring amino acids described above. It is further understood that the term unnatural amino acid includes homologues of the natural amino acids, and synthetically modified forms of the natural amino acids. The synthetically modified forms include, but are not limited to, amino acids having alkylene chains shortened or lengthened by up to two carbon atoms, amino acids comprising optionally substituted aryl groups, and amino acids comprised halogenated groups, preferably halogenated alkyl and aryl groups. When attached to a linker or conjugate of the invention, the amino acid is in the form of an “amino acid side chain”, where the carboxylic acid group of the amino acid has been replaced with a keto (C(O)) group. Thus, for example, an alanine side chain is —C(O)—CH(NH₂)—CH₃, and so forth.

The peptide sequence (AA¹)_{c} is functionally the amidification residue of a single amino acid (when c=1) or a plurality of amino acids joined together by amide bonds. The peptide sequence (AA¹)_{c} preferably is selected for enzyme-catalyzed cleavage by an enzyme in a location of interest in a biological system. For example, for conjugates that are targeted to but not internalized by a cell, a peptide is chosen that is cleaved by a protease that in the extracellular matrix, e.g., a protease released by nearby dying cells or a tumor-associated protease, such that the peptide is cleaved extracellularly. For conjugates that are designed for internalization by a cell, the sequence (AA¹)_{c} preferably is selected for cleavage by an endosomal or lysosomal protease. The number of amino acids within the peptide can range from 1 to 20; but more preferably there will be 1-8 amino acids, 1-6 amino acids or 1, 2, 3 or 4 amino acids comprising (AA¹)_{c}. Peptide sequences that are susceptible to cleavage by specific enzymes or classes of enzymes are well known in the art.

Preferably, (AA¹)_{c} contains an amino acid sequence (“cleavage recognition sequence”) that is a cleavage site by the protease. Many protease cleavage sequences are known in the art. See, e.g., Matayoshi et al. Science 247: 954 (1990); Dunn et al. Meth. Enzymol. 241: 254 (1994); Seidah et al. Meth. Enzymol. 244: 175 (1994); Thornberry, Meth. Enzymol. 244: 615 (1994); Weber et al. Meth. Enzymol. 244: 595 (1994); Smith et al. Meth. Enzymol. 244: 412 (1994); Bouvier et al. Meth. Enzymol. 248: 614 (1995), Hardy et al., in Amyloid Protein Precursor in Development, Aging, and Alzheimer's Disease, ed. Masters et al. pp. 190-198 (1994).

The peptide typically includes 3-12 (or more) amino acids. The selection of particular amino acids will depend, at least in part, on the enzyme to be used for cleaving the peptide, as well as, the stability of the peptide in vivo. One example of a suitable cleavable peptide is β-Ala-Leu-Ala-Leu (SEQ ID NO: 27). This can be combined with a stabilizing group to form succinyl-β-Ala-Leu-Ala-Leu (SEQ ID NO: 30). Other examples of suitable cleavable peptides are provided in the references cited below. Alternatively, linkers comprising a single amino acid residue can be used, as disclosed in WO 2008/103693, the disclosure of which is incorporated herein by reference.

In a preferred embodiment, the peptide sequence (AA¹)_{c} is chosen based on its ability to be cleaved by a lysosomal proteases, examples of which include cathepsins B, C, D, H, L and S. Preferably, the peptide sequence (AA¹)_{c} is capable of being cleaved by cathepsin B in vitro. Though cathepsin B is a lysosomal proteaste, it is believed that a certain concentration of it is found in the extracellular matrix surrounding tumor tissues.

In another embodiment, the peptide sequence (AA¹)_{c} is chosen based on its ability to be cleaved by a tumor-associated protease, such as a protease found extracellularly in the vicinity of tumor cells, examples of which include thimet oligopeptidase (TOP) and CD10. Or, the sequence (AA¹)_{c} is designed for selective cleavage by urokinase or tryptase.

As one illustrative example, CD10, also known as neprilysin, neutral endopeptidase (NEP), and common acute lymphoblastic leukemia antigen (CALLA), is a type II cell-surface zinc-dependent metalloprotease. Cleavable substrates suitable for use with CD10 include Leu-Ala-Leu and Ile-Ala-Leu.

Another illustrative example is based on matrix metalloproteases (MMP). Probably the best characterized proteolytic enzymes associated with tumors, there is a clear correlation of activation of MMPs within tumor microenvironments. In particular, the soluble matrix enzymes MMP2 (gelatinase A) and MMP9 (gelatinase B), have been intensively studied, and shown to be selectively activated during tissue remodeling including tumor growth. Peptide sequences designed to be cleaved by MMP2 and MMP9 have been designed and tested for conjugates of dextran and methotrexate (Chau et al., Bioconjugate Chem. 15:931-941 (2004)); PEG (polyethylene glycol) and doxorubicin (Bae et al., Drugs Exp. Clin. Res. 29:15-23 (2004)); and albumin and doxorubicin (Kratz et al., Bioorg. Med. Chem. Lett. 11:2001-2006 (2001)). Examples of suitable sequences for use with MMPs include, but are not limited to, Pro-Val-Gly-Leu-Ile-Gly (SEQ. ID NO: 21), Gly-Pro-Leu-Gly-Val (SEQ. ID NO: 22), Gly-Pro-Leu-Gly-Ile-Ala-Gly-Gln (SEQ. ID NO: 23), Pro-Leu-Gly-Leu (SEQ. ID NO: 24), Gly-Pro-Leu-Gly-Met-Leu-Ser-Gln (SEQ. ID NO: 25), and Gly-Pro-Leu-Gly-Leu-Trp-Ala-Gln (SEQ. ID NO: 26). (See, e.g., the previously cited references as well as Kline et al., Mol. Pharmaceut. 1:9-22 (2004) and Liu et al., Cancer Res. 60:6061-6067 (2000).)

Yet another example is type II transmembrane serine proteases. This group of enzymes includes, for example, hepsin, testisin, and TMPRSS4. Gln-Ala-Arg is one substrate sequence that is useful with matriptase/MT-SP1 (which is over-expressed in breast and ovarian cancers) and Leu-Ser-Arg is useful with hepsin (over-expressed in prostate and some other tumor types). (See, e.g., Lee et. al., J. Biol. Chem. 275:36720-36725 and Kurachi and Yamamoto, Handbook of Proeolytic Enzymes Vol. 2, 2^{nd} edition (Barrett A J, Rawlings N D & Woessner J F, eds) pp. 1699-1702 (2004).)

Suitable, but non-limiting, examples of peptide sequences suitable for use in the conjugates of the invention include Val-Cit, Cit-Cit, Val-Lys, Phe-Lys, Lys-Lys, Ala-Lys, Phe-Cit, Leu-Cit, Ile-Cit, Trp, Cit, Phe-Ala, Phe-N⁹-tosyl-Arg, Phe-N⁹-nitro-Arg, Phe-Phe-Lys, D-Phe-Phe-Lys, Gly-Phe-Lys, Leu-Ala-Leu, Ile-Ala-Leu, Val-Ala-Val, Ala-Len-Ala-Leu, β-Ala-Leu-Ala-Leu (SEQ ID NO: 27), Gly-Phe-Leu-Gly (SEQ. ID NO: 28), Val-Ala, Leu-Leu-Gly-Leu (SEQ ID NO: 29), Leu-Asn-Ala, and Lys-Leu-Val. Preferred peptides sequences are Val-Cit and Val-Lys.

In another embodiment, the amino acid located the closest to the drug moiety is selected from the group consisting of: Ala, Asn, Asp, Cit, Cys, Gln, Gln, Gly, Ile, Len, Lys, Met, Phe, Pro, Ser, Thr, Trp, Tyr, and Val. In yet another embodiment, the amino acid located the closest to the drug moiety is selected from the group consisting of: Ala, Asn, Asp, Cys, Gln, Gln, Gly, Ile, Leu, Met, Phe, Pro, Ser, Thr, Trp, Tyr, and Val.

One of skill in the art can readily evaluate an array of peptide sequences to determine their utility in the present invention without resort to undue experimentation. See, for example, Zimmerman, M., et al., (1977) Analytical Biochemistry 78:47-51; Lee, D., et al., (1999) Bioorganic and Medicinal Chemistry Letters 9:1667-72; and Rano, T. A., et al., (1997) Chemistry and Biology 4:149-55.

A conjugate of this invention may optionally contain two or more linkers. These linkers may be the same or different. For example, a peptidyl linker may be used to connect the drug to the ligand and a second peptidyl linker may attach a diagnostic agent to the complex. Other uses for additional linkers include linking analytical agents, biomolecules, targeting agents, and detectable labels to the antibody-partner complex.

### Hydrazine Linkers (H)

In another embodiment, the conjugate of the invention comprises a hydrazine self-immolative linker, wherein the conjugate has the structure:

X⁴-(L⁴)ₚ-H-(L¹)ₘ-D

wherein D, L¹, L⁴, p, m, and X⁴ are as defined above and described further herein, and H is a linker comprising the structure:

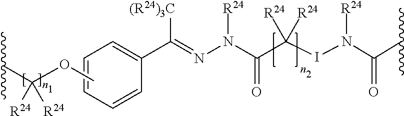

wherein n₁ is an integer from 1-10; n₂ is 0, 1, or 2; each R²⁴ is a member independently selected from the group consisting of H, substituted alkyl, unsubstituted alkyl, substituted heteroalkyl, and unsubstituted heteroalkyl; and I is either a bond (i.e., the bond between the carbon of the backbone and the adjacent nitrogen) or:

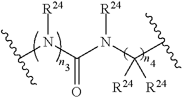

wherein n₃ is 0 or 1, with the proviso that when n₃ is 0, n₂ is not 0; and n₄ is 1, 2, or 3.

In one embodiment, the substitution on the phenyl ring is a para substitution. In preferred embodiments, n₁ is 2, 3, or 4 or n₁ is 3. In preferred embodiments, n₂ is 1. In preferred embodiments, I is a bond (i.e., the bond between the carbon of the backbone and the adjacent nitrogen). In one aspect, the hydrazine linker, H, can form a 6-membered self immolative linker upon cleavage, for example, when n₃ is 0 and n₄ is 2. In another aspect, the hydrazine linker, H, can form two 5-membered self immolative linkers upon cleavage. In yet other aspects, H forms a 5-membered self immolative linker, H forms a 7-membered self immolative linker, or H forms a 5-membered self immolative linker and a 6-membered self immolative linker, upon cleavage. The rate of cleavage is affected by the size of the ring formed upon cleavage. Thus, depending upon the rate of cleavage desired, an appropriate size ring to be formed upon cleavage can be selected.

Another hydrazine structure, H, has the formula:

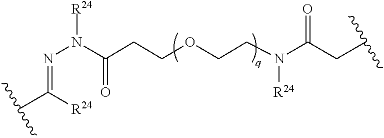

where q is 0, 1, 2, 3, 4, 5, or 6; and each R²⁴ is a member independently selected from the group consisting of H, substituted alkyl, unsubstituted alkyl, substituted heteroallyl, and unsubstituted heteroallyl. This hydrazine structure can also form five-, six-, or seven-membered rings and additional components can be added to form multiple rings.

The preparation, cleavage chemistry and cyclization kinetics of the various hydrazine linkers is disclosed in WO 2005/112919, the disclosure of which is incorporated herein by reference.

### Disulfide Linkers (J)

In yet another embodiment, the linker comprises an enzymatically cleavable disulfide group. In one embodiment, the invention provides a cytotoxic antibody-partner compound having a structure according to Formula (d):

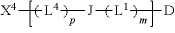

wherein D, L¹, L⁴, p, m, and X⁴ are as defined above and described further herein, and J is a disulfide linker comprising a group having the structure:

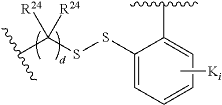

wherein each R²⁴ is a member independently selected from the group consisting of H, substituted alkyl, unsubstituted alkyl, substituted heteroalkyl, and unsubstituted heteroalkyl; each K is a member independently selected from the group consisting of substituted alkyl, unsubstituted alkyl, substituted heteroalkyl, unsubstituted heteroalkyl, substituted aryl, unsubstituted aryl, substituted heteroaryl, unsubstituted heteroaryl, substituted heterocycloalkyl, unsubstituted heterocycloalkyl, halogen, NO₂, NR²¹R²², NR²¹COR²², OCONR²¹R²², OCOR²¹, and OR²¹ wherein R²¹ and R²² are independently selected from the group consisting of H, substituted alkyl, unsubstituted alkyl, substituted heteroalkyl, unsubstituted heteroalkyl, substituted aryl, unsubstituted aryl, substituted heteroaryl, unsubstituted heteroaryl, substituted heterocycloalkyl and unsubstituted heterocycloalkyl; i is an integer of 0, 1, 2, 3, or 4; and d is an integer of 0, 1, 2, 3, 4, 5, or 6.

The aromatic ring of a disulfide linker can be substituted with one or more “K” groups. A “K” group is a substituent that replaces a hydrogen otherwise attached to one of the four non-substituted carbons that are part of the ring structure. The “K” group may be a single atom, such as a halogen, or may be a multi-atom group, such as alkyl, heteroalkyl, amino, nitro, hydroxy, alkoxy, haloalkyl, and cyano. Exemplary K substituents include, but are not limited to, F, Cl, Br, I, NO₂, OH, OCH₃, NHCOCH₃, N(CH₃)₂, NHCOCF₃ and methyl. For “Kᵢ”, i is an integer of 0, 1, 2, 3, or 4. In a specific embodiment, i is 0.

In a preferred embodiment, the linker comprises an enzymatically cleavable disulfide group of the following formula:

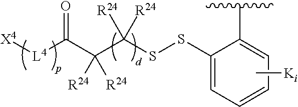

wherein L⁴, X⁴, p, and R²⁴ are as described above, and d is 0, 1, 2, 3, 4, 5, or 6. In a particular embodiment, d is 1 or 2.

A more specific disulfide linker is shown in the formula below:

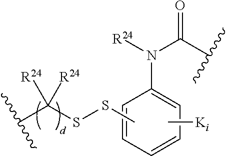

Preferably, d is 1 or 2 and each K is H.

Another disulfide linker is shown in the formula below:

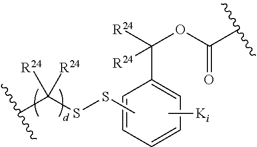

Preferably, d is 1 or 2 and each K is H.

In various embodiments, the disulfides are ortho to the amine. In another specific embodiment, a is 0. In preferred embodiments, R²⁴ is independently selected from H and CH₃.

The preparation and use of disulfide linkers such as those described above is disclosed in WO 2005/112919, the disclosure of which is incorporated herein by reference.

For further discussion of types of cytotoxins, linkers and the conjugation of therapeutic agents to antibodies, see also U.S. Pat. No. 7,087,600; U.S. Pat. No. 6,989,452; U.S. Pat. No. 7,129,261; US 2006/0004081; US 2006/0247295; WO 02/096910; WO 2007/051081; WO 2005/112919; WO 2007/059404; WO 2008/083312; WO 2008/103693; Saito et al. (2003) Adv. Drug Deliv. Rev. 55:199-215; Trail et al. (2003) Cancer Immunol. Immunother. 52:328-337; Payne. (2003) Cancer Cell 3:207-212; Allen (2002) Nat. Rev. Cancer 2:750-763; Pastan and Kreitman (2002) Curr. Opin. Investig. Drugs 3:1089-1091; Senter and Springer (2001) Adv. Drug Deliv. Rev. 53:247-264, each of which is hereby incorporated by reference.

### Cytotoxins as Partner Molecules

In one aspect, the present invention features an antibody conjugated to a partner molecule, such as a cytotoxin, a drug (e.g., an immunosuppressant) or a radiotoxin. Such conjugates are also referred to as “immunotoxins.” A cytotoxin or cytotoxic agent includes any agent that is detrimental to (e.g., kills) cells. Herein, “cytotoxin” includes compounds that are in a prodrug form and are converted in vivo to the actual toxic species.

Examples of partner molecules of the present invention include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Examples of partner molecules also include, for example, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclophosphamide, busulfan, tubulysin, dibromomannitol, streptozotocin, mitomycin C, cisplatin, anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). Other preferred examples of partner molecules that can be conjugated to an antibody of the invention include calicheamicins, maytansines and auristatins, and derivatives thereof.

Preferred examples of partner molecule are analogs and derivatives of CC-1065 and the structurally related duocarmycins. Despite its potent and broad antitumor activity, CC-1065 cannot be used in humans because it causes delayed death in experimental animals, prompting a search for analogs or derivatives with a better therapeutic index.

Many analogues and derivatives of CC-1065 and the duocannycins are known in the art. The research into the structure, synthesis and properties of many of the compounds has been reviewed. See, for example, Boger et al., Angew. Chem. Int. Ed. Engl. 35: 1438 (1996); and Boger et al., Chem. Rev. 97: 787 (1997). Other disclosures relating to CC-1065 analogs or derivatives include: U.S. Pat. No. 5,101,038; U.S. Pat. No. 5,641,780; U.S. Pat. No. 5,187,186; U.S. Pat. No. 5,070,092; U.S. Pat. No. 5,703,080; U.S. Pat. No. 5,070,092; U.S. Pat. No. 5,641,780; U.S. Pat. No. 5,101,038; U.S. Pat. No. 5,084,468; U.S. Pat. No. 5,739,350; U.S. Pat. No. 4,978,757, U.S. Pat. No. 5,332,837 and U.S. Pat. No. 4,912,227; WO 96/10405; and EP 0,537,575 A1

In a particularly preferred aspect, the partner molecule is a CC-1065/duocarmycin analog having a structure according to the following formula (e):

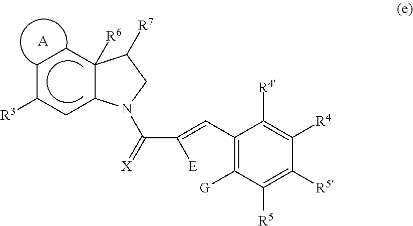

in which ring system A is a member selected from substituted or unsubstituted aryl substituted or unsubstituted heteroaryl and substituted or unsubstituted heterocycloalkyl groups. Exemplary ring systems A include phenyl and pyrrole.

The symbols E and G are independently selected from H, substituted or unsubstituted alkyl, substituted or unsubstituted heteroalkyl, a heteroatom, a single bond or E and G are optionally joined to form a ring system selected from substituted or unsubstituted aryl, substituted or unsubstituted heteroaryl and substituted or unsubstituted heterocycloalkyl.

The symbol X represents a member selected from O, S and NR²³. R²³ is a member selected from H, substituted or unsubstituted alkyl, substituted or unsubstituted heteroalkyl, and acyl.

The symbol R³ represents a member selected from (═O), SR¹¹, NHR¹¹ and OR¹¹, in which R¹¹ is H, substituted or unsubstituted alkyl, substituted or unsubstituted heteroalkyl, monophosphates, diphosphates, triphosphates, sulfonates, acyl, C(O)R¹²R¹³, C(O)OR¹², C(O)NR¹²R¹³, P(O)(OR¹²)₂, C(O)CHR¹²R¹³, SR¹² or SiR¹²R¹³R¹⁴. The symbols R¹², R¹³, and R¹⁴ independently represent H, substituted or unsubstituted alkyl, substituted or unsubstituted heteroalkyl and substituted or unsubstituted aryl, where R¹² and R¹³ together with the nitrogen or carbon atom to which they are attached are optionally joined to form a substituted or unsubstituted heterocycloalkyl ring system having from 4 to 6 members, optionally containing two or more heteroatoms.

R⁴, R⁴′, R⁵ and R⁵′ are members independently selected from H, substituted or unsubstituted alkyl, substituted or unsubstituted aryl, substituted or unsubstituted heteroaryl, substituted or unsubstituted heterocycloalkyl, halogen, NO₂, NR¹⁵R¹⁶, NC(O)R¹⁵, OC(O)NR¹⁵R¹⁶, OC(O)OR¹⁵, C(O)R¹⁵, SR¹⁵, OR¹⁵, CR¹⁵═NR¹⁶, and O(CH₂)ₙN(CH₃)₂, where n is an integer from 1 to 20, or any adjacent pair of R⁴, R⁴′, R⁵ and R⁵′, together with the carbon atoms to which they are attached, are joined to form a substituted or unsubstituted cycloalkyl or heterocycloalkyl ring system having from 4 to 6 members. R¹⁵ and R¹⁶ independently represent H, substituted or unsubstituted alkyl, substituted or unsubstituted hetero-alkyl, substituted or unsubstituted aryl, substituted or unsubstituted heteroaryl, substituted or unsubstituted heterocycloalkyl and substituted or unsubstituted peptidyl, where R¹⁵ and R¹⁶ together with the nitrogen atom to which they are attached are optionally joined to form a substituted or unsubstituted heterocycloalkyl ring system having from 4 to 6 members, optionally containing two or more heteroatoms. One exemplary structure is aniline.

One of R³, R⁴, R⁴′, R⁵, and R⁵′ joins the cytotoxin to a linker or enzyme cleavable substrate of the present invention, as described herein, for example to L¹ or L³, if present or to F, H, or J.

R⁶ is a single bond which is either present or absent. When R⁶ is present, R⁶ and R⁷ are joined to form a cyclopropyl ring. R⁷ is CH₂—X¹ or —CH₂—. When R⁷ is —CH₂— it is a component of the cyclopropane ring. The symbol X¹ represents a leaving group such as a halogen, for example Cl, Br or F. The combinations of R⁶ and R⁷ are interpreted in a manner that does not violate the principles of chemical valence.

X¹ may be any leaving group. Useful leaving groups include, but are not limited to, halogens, azides, sulfonic esters (e.g., alkylsulfonyl, arylsulfonyl), oxonium ions, alkyl perchlorates, ammonioalkanesulfonate esters, alkylfluorosulfonates and fluorinated compounds (e.g., triflates, nonaflates, tresylates) and the like. Particular halogens useful as leaving groups are F, Cl and Br.

The curved line within the six-membered ring indicates that the ring may have one or more degrees of unsaturation, and it may be aromatic. Thus, ring structures such as those set forth below, and related structures, are within the scope of Formula (f):

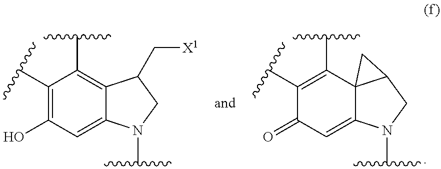

In one embodiment, R¹¹ includes a moiety, X⁵, that does not self-cyclize and links the drug to L¹ or L³, if present, or to F, H, or J. The moiety, X⁵, is preferably cleavable using an enzyme and, when cleaved, provides the active drug. As an example, R¹¹ can have the following structure (with the right side coupling to the remainder of the drug):

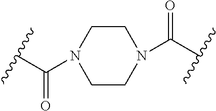

In some embodiments, at least one of R⁴, R⁴′, R⁵, and R⁵′ links said drug to L¹, if present, or to F, H, J, or X², and R³ is selected from SR¹¹, NHR¹¹ and OR¹¹. R¹¹ is selected from —SO(OH)₂, —PO(OH)₂, -AAₙ, —Si(CH₃)₂C(CH₃)₃, —C(O)OPhNH(AA)ₘ,

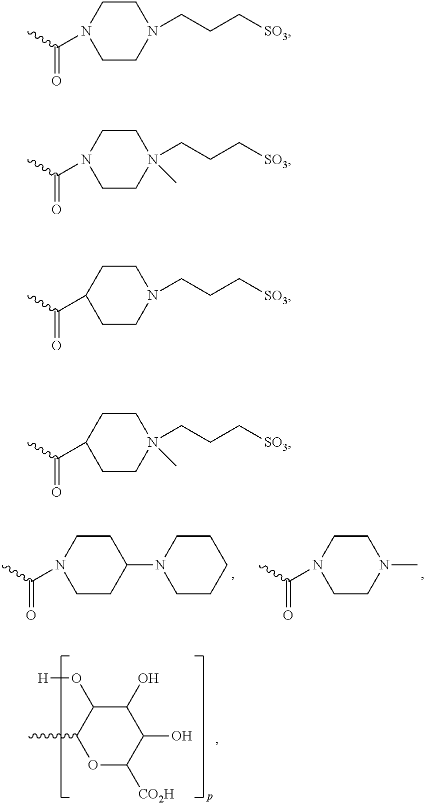

or any other sugar or combination of sugars

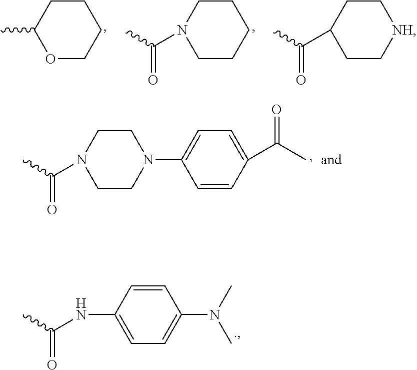

and pharmaceutically acceptable salts thereof, where n is any integer in the range of 1 to 10, m is any integer in the range of 1 to 4, p is any integer in the range of 1 to 6, and AA is any natural or non-natural amino acid. Where the compound of formula (e) is conjugated via R⁴, R⁴′, R⁵, or R⁶, R³ preferably comprises a cleavable blocking group whose presence blocks the cytotoxic activity of the compound but is cleavable under conditions found at the intended site of action by a mechanism different from that for cleavage of the linker conjugating the cytotoxin to the antibody. In this way, if there is adventitious cleavage of the conjugate in the plasma, the blocking group attenuates the cytotoxicity of the released cytotoxin. For instance, if the conjugate has a hydrazone or disulfide linker, the blocking group can be an enzymatically cleavable amide. Or, if the linker is a peptidyl one cleavable by a protease, the blocking group can be an ester or carbamate cleavable by a carboxyesterase.

For example, in a preferred embodiment, D is a cytotoxin having a structure (j):

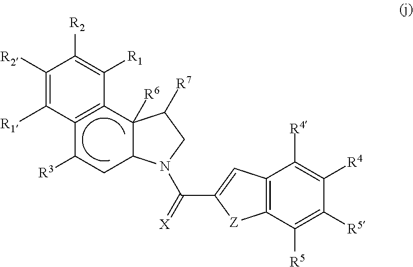

In this structure, R³, R⁶, R⁷, R⁴, R⁴′, R⁵, R⁵′ and X are as described above for Formula (e). Z is a member selected from O, S and NR²³, where R²³ is a member selected from H, substituted or unsubstituted alkyl, substituted or unsubstituted heteroalkyl, and acyl.

R¹ is H, substituted or unsubstituted lower alkyl, C(O)R⁸, or CO₂R⁸, wherein R⁸ is a member selected from NR⁹R¹⁰ and OR⁹, in which R⁹ and R¹⁰ are members independently selected from H, substituted or unsubstituted alkyl and substituted or unsubstituted heteroalkyl.

R^{1′} is H, substituted or unsubstituted lower alkyl, or C(O)R⁸, wherein R⁸ is a member selected from NR⁹R¹⁰ and OR⁹, in which R⁹ and R¹⁰ are members independently selected from H, substituted or unsubstituted alkyl and substituted or unsubstituted heteroalkyl.

R² is H, or substituted or unsubstituted lower alkyl or unsubstituted heteroalkyl or cyano or alkoxy; and R^{2′} is H, or substituted or unsubstituted lower alkyl or unsubstituted heteroalkyl.

One of R³, R⁴, R⁴′, R⁵, or R⁵′ links the cytotoxin to L¹ or L³, if present, or to F, H, or J.

A further embodiment has the formula:

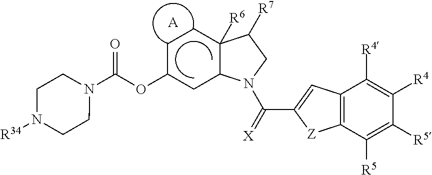

In this structure, A, R⁶, R⁷, X, R⁴, R^{4′}, R⁵, and R^{5′} are as described above for Formula (e). Z is a member selected from O, S and NR²³, where R²³ is a member selected from H, substituted or unsubstituted alkyl, substituted or unsubstituted heteroalkyl, and acyl;

R³⁴ is C(═O)R³³ or C₁-C₆ alkyl, where R³³ is selected from H, substituted or unsubstituted alkyl, substituted or unsubstituted aryl, substituted or unsubstituted heteroaryl, substituted or unsubstituted heterocycloalkyl, halogen, NO₂, NR¹⁵R¹⁶, NC(O)R¹⁵, OC(O)NR¹⁵R¹⁶, OC(O)OR¹⁵, C(O)R¹⁵, SR¹⁵, OR¹⁵, CR¹⁵═NR¹⁶, and O(CH₂)ₙN(CH₃)₂, where n is an integer from 1 to 20. R¹⁵ and R¹⁶ independently represent H, substituted or unsubstituted alkyl, substituted or unsubstituted heteroalkyl, substituted or unsubstituted aryl, substituted or unsubstituted heteroaryl, substituted or unsubstituted heterocycloalkyl and substituted or unsubstituted peptidyl, where R¹⁵ and R¹⁶ together with the nitrogen atom to which they are attached are optionally joined to form a substituted or unsubstituted heterocycloalkyl ring system having from 4 to 6 members, optionally containing two or more heteroatoms.

Preferably, A is substituted or unsubstituted phenyl or substituted or unsubstituted pyrrole. Further, any selection of substituents described herein for R¹¹ is also applicable to R³³.

A preferred partner molecule has a structure represented by formula (I)

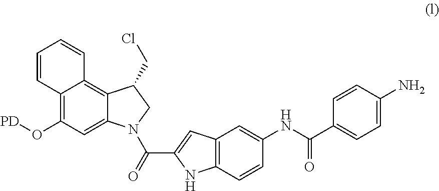

In formula (I), PD represents a prodrugging group (sometimes also referred to as a protecting group). Compound (I) is hydrolyzed in situ (preferably enzymatically) to release the compound of formula (II). As those skilled in the art will recognize, compound (II) belongs to the class of compounds known as CBI compounds (Boger et al., J. Org. Chem. 2001, 66, 6654-6661 and Boger et al., US 2005/0014700 A1 (2005). CBI compounds are converted in situ (or, when administered to a patient, in vivo) to their cyclopropyl derivatives such as compound (III), bind to the minor groove of DNA, and then alkylate DNA on an adenine group, with the cyclopropyl derivative believed to be the actual alkylating species.

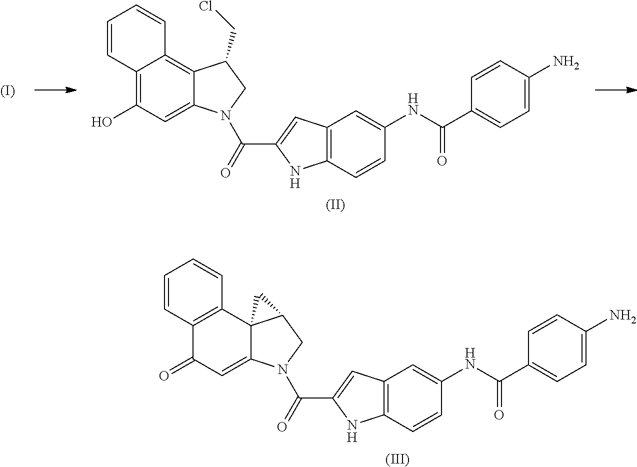

Non-limiting examples of suitable prodrugging groups PD include esters, carbamates, phosphates, and glycosides, as illustrated following:

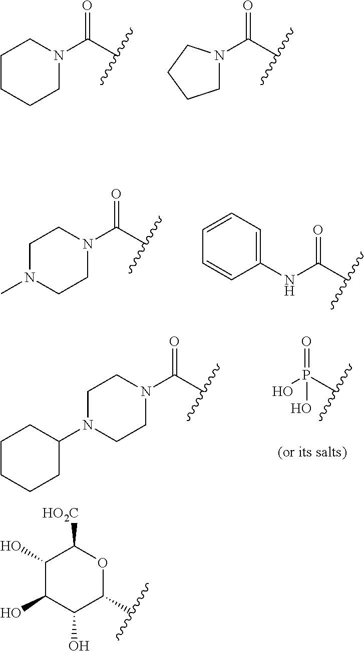

Preferred prodrugging groups PD are carbamates (exemplified by the first five structures above), which are hydrolyzable by carboxyesterases; phosphates (the sixth structure above), which are hydrolyzable by alkaline phosphatase, and β-glucuronic acid derivatives, which are hydrolyzable by β-glucuronidase. A specific preferred partner molecule is a carbamate prodrugged one, represented by formula (IV):

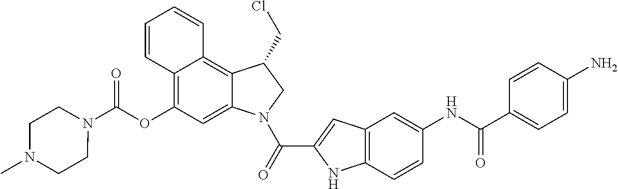

### Markers as Partner Molecules

Where the partner molecule is a marker, it can be any moiety having or generating a detectable physical or chemical property, thereby indicating its presence in a particular tissue or cell. Markers (sometimes also called reporter groups) have been well developed in the area of immunoassays, biomedical research, and medical diagnosis. A marker may be detected by spectroscopic, photochemical, biochemical, immunochemical, electrical, optical or chemical means. Examples include magnetic beads (e.g., DYNABEADS™), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic beads (e.g., polystyrene, polypropylene, latex, etc.).

The marker is preferably a member selected from the group consisting of radioactive isotopes, fluorescent agents, fluorescent agent precursors, chromophores, enzymes and combinations thereof. Examples of suitable enzymes are horseradish peroxidase, alkaline phosphatase, β-galactosidase, and glucose oxidase. Fluorescent agents include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal producing systems that may be used, see U.S. Pat. No. 4,391,904.

Markers can be attached by indirect means: a ligand molecule (e.g., biotin) is covalently bound to an antibody. The ligand then binds to another molecule (e.g., streptavidin), which is either inherently detectable or covalently bound to a signal system, such as a detectable enzyme, a fluorescent compound, or a chemiluminescent compound.

### Examples of Conjugates

Specific examples of partner molecule-linker combinations suitable for conjugation to an antibody of this invention are shown following:

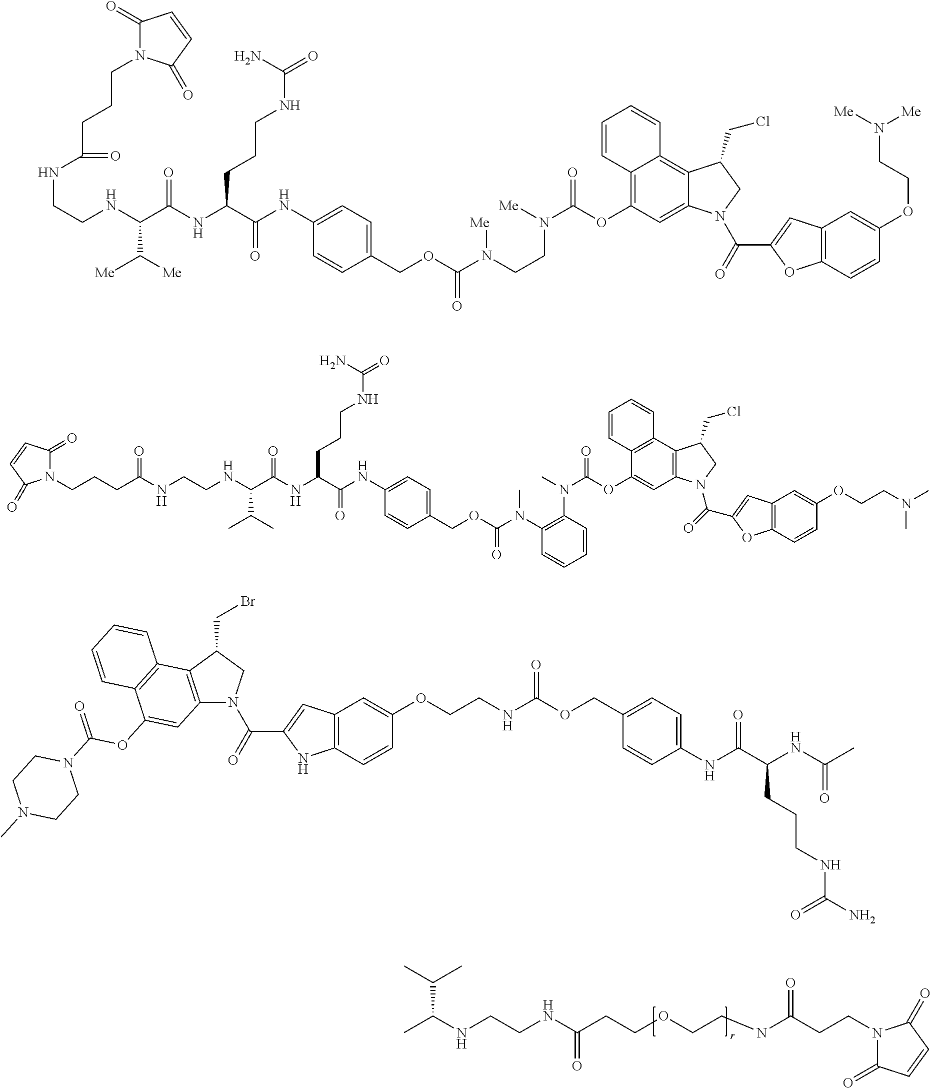

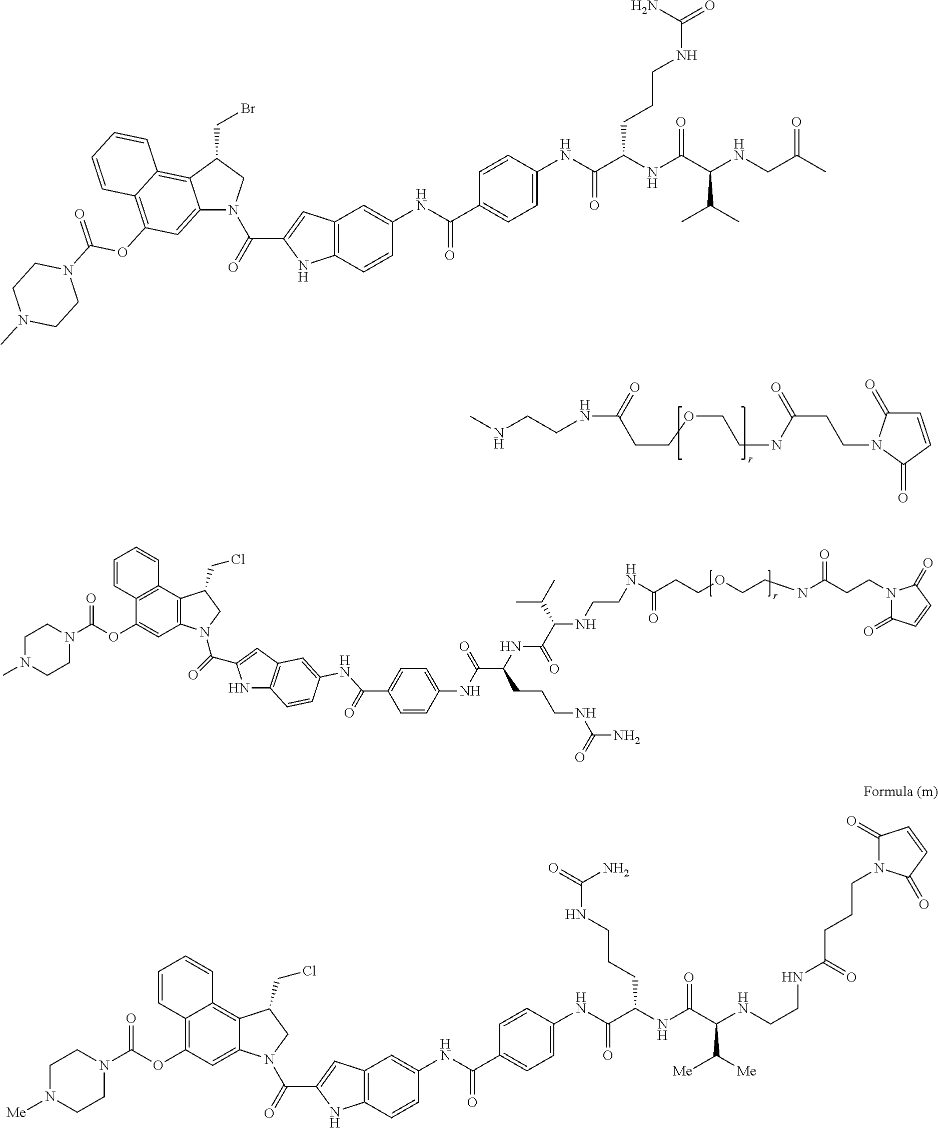

### Toxin B

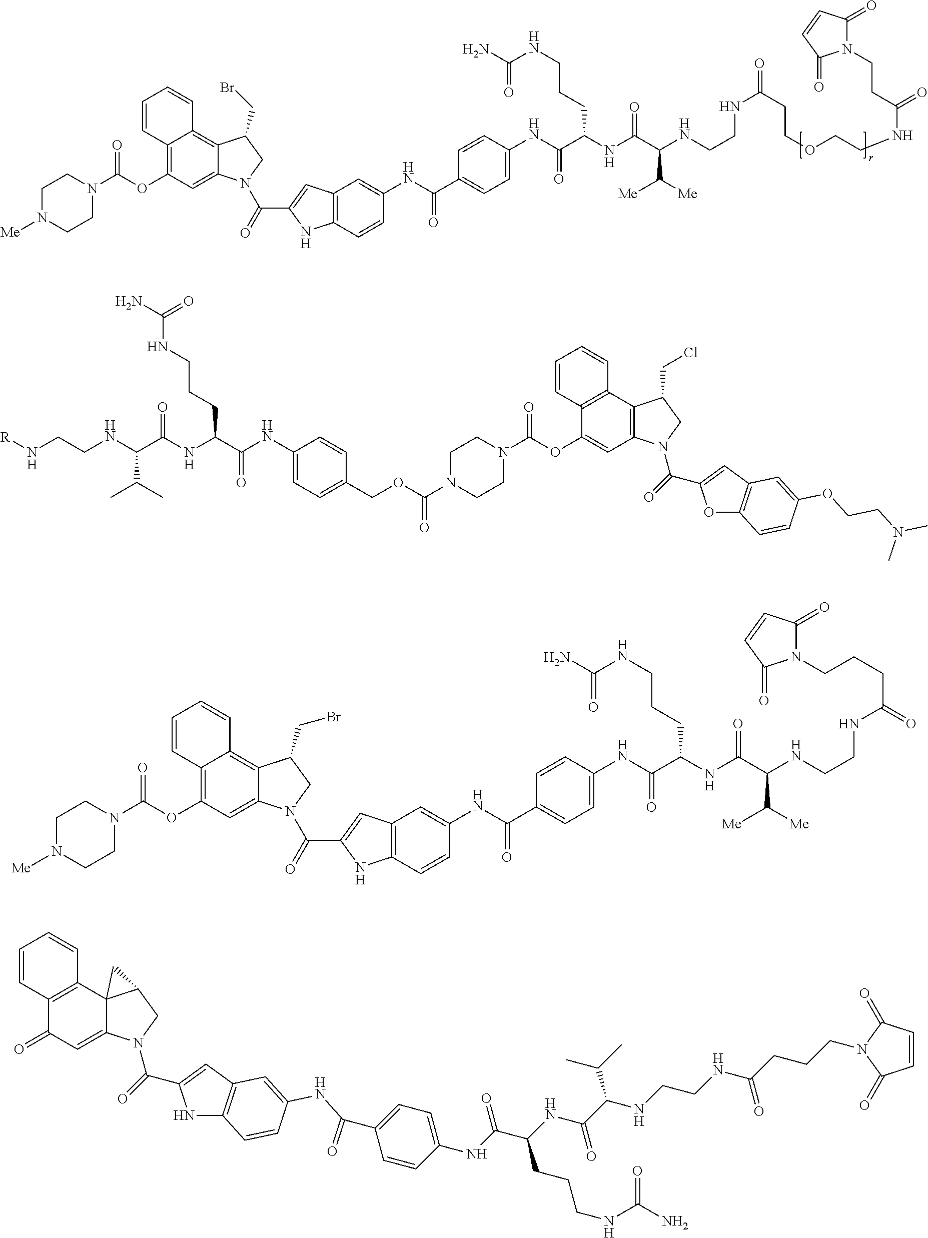

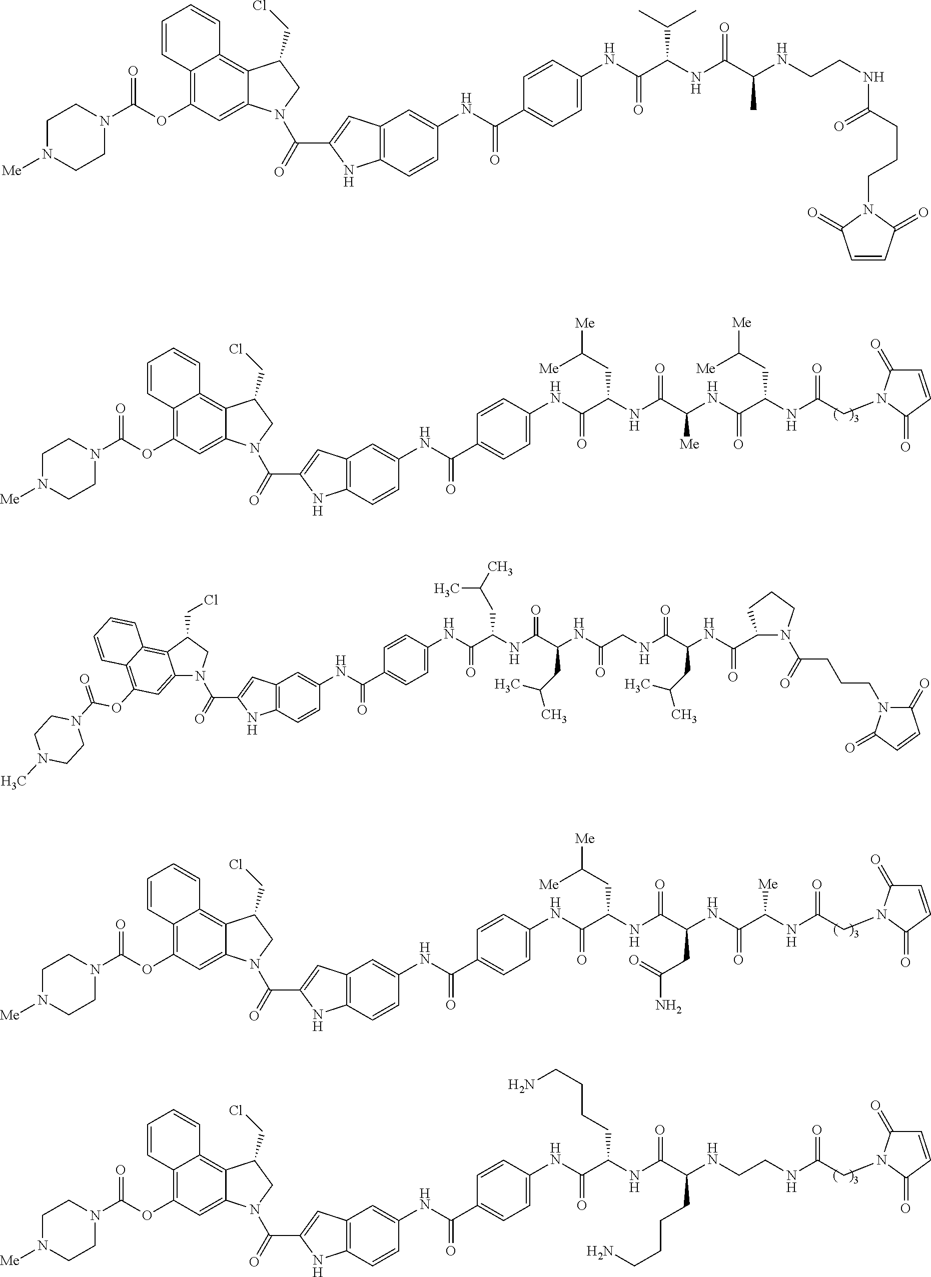

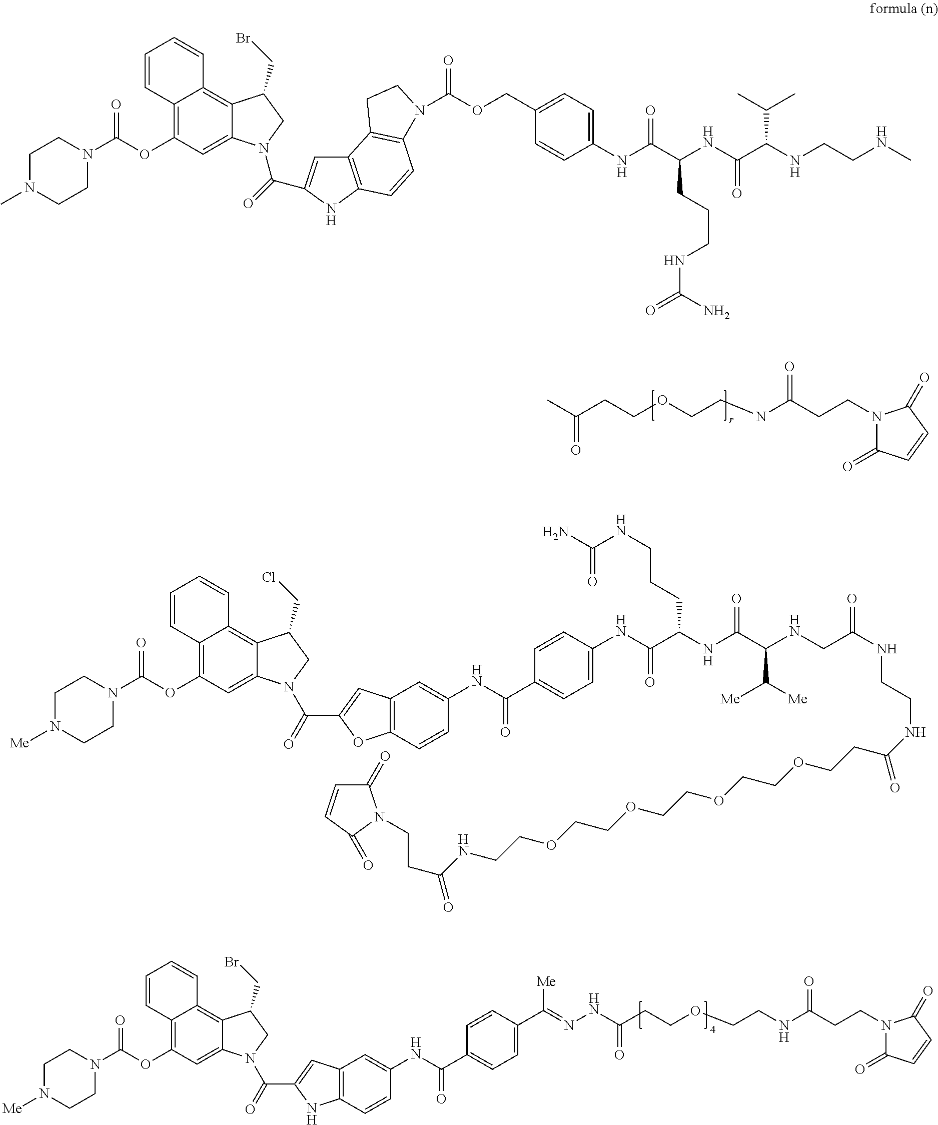

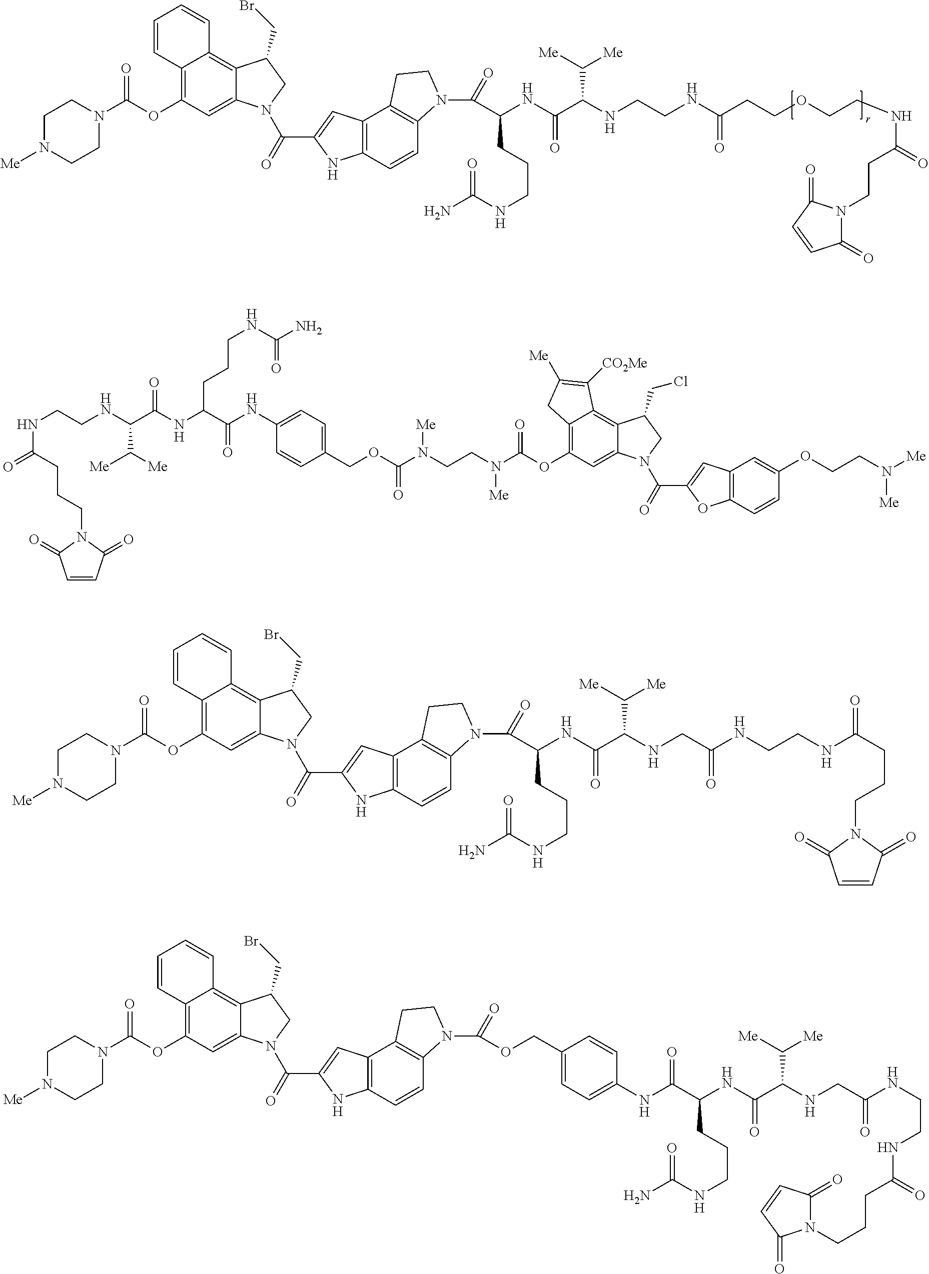

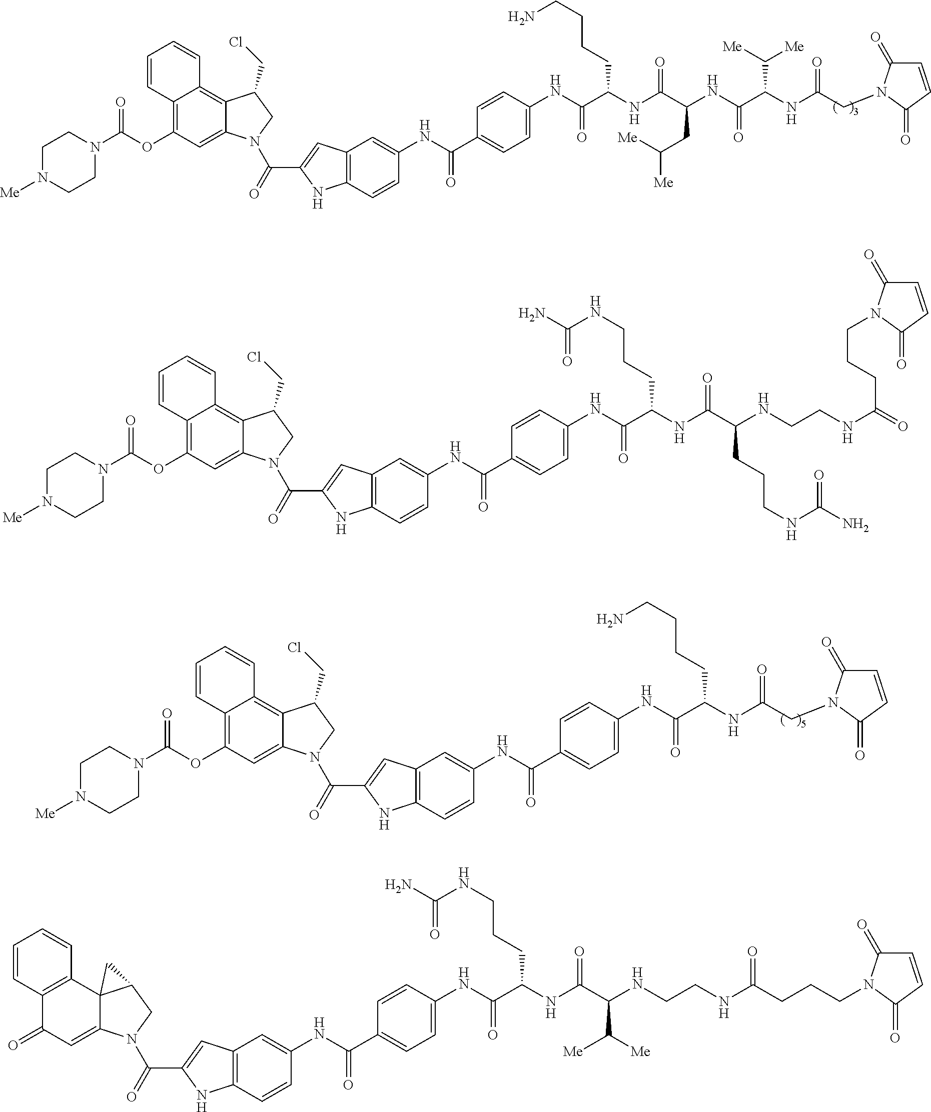

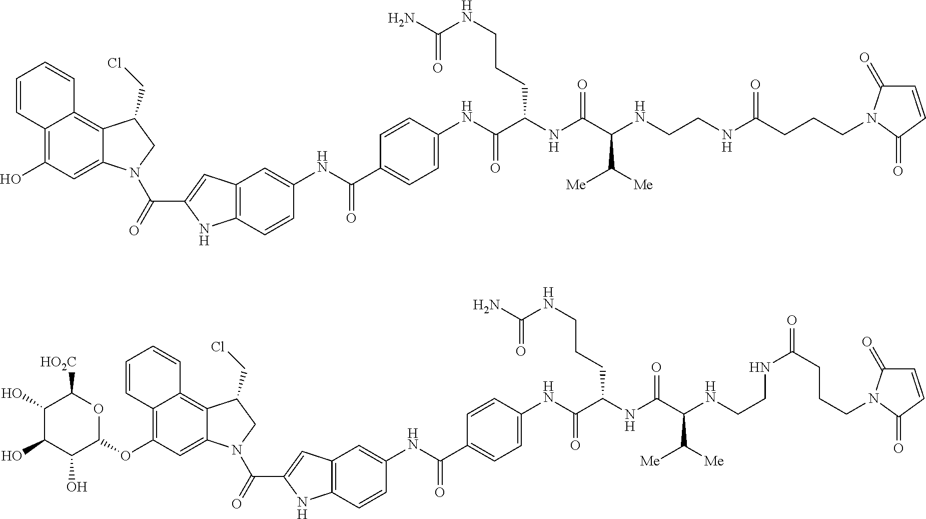

In the foregoing compounds, where the subscript r is present in a formula, it is an integer in the range of 0 to 24, preferably 4. R, wherever it occurs, is

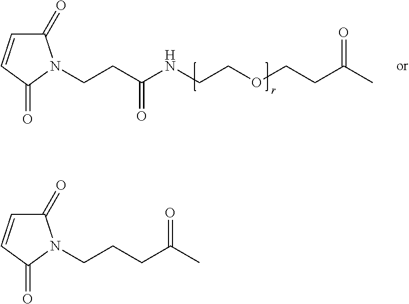

Each of the foregoing compounds has a maleimide group and is ready for conjugation to an antibody via a sulfhydryl group thereon.

### Pharmaceutical Compositions

In another aspect, the present disclosure provides a composition, e.g., a pharmaceutical composition, containing one or a combination of monoclonal antibodies, or antigen-binding portion(s) thereof, of the present disclosure, formulated together with a pharmaceutically acceptable carrier. Such compositions may include one or a combination of (e.g., two or more different) antibodies, or immunoconjugates or bispecific molecules of this disclosure. For example, a pharmaceutical composition of this disclosure can comprise a combination of antibodies (or immunoconjugates or bispecifics) that bind to different epitopes on the target antigen or that have complementary activities.

Pharmaceutical compositions of this disclosure also can be administered in combination therapy, i.e., combined with other agents. For example, the combination therapy can include an anti-B7-H4 antibody of the present disclosure combined with at least one other anti-cancer agent. Examples of therapeutic agents that can be used in combination therapy are described in greater detail below in the section on uses of the antibodies of this disclosure.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound, i.e., antibody, immunoconjugate, or bispecific molecule, may be coated in a material to protect the compound from the action of acids and other natural conditions that may inactivate the compound.

The pharmaceutical compounds of this disclosure may include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition of this disclosure also may include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and nonaqueous carriers that may be employed in the pharmaceutical compositions of this disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of this disclosure is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable earner.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of this disclosure are dictated by and directly dependent on (a) the unique characteristics of the active compound and the particular therapeutic effect to be achieved, and (b) the limitations inherent in the art of compounding such an active compound for the treatment of sensitivity in individuals.

For administration of the antibody, the dosage ranges from about 0.0001 to 100 mg/kg, and more usually 0.01 to 5 mg/kg, of the host body weight. For example dosages can be 0.3 mg/kg body weight, 1 mg/kg body weight, 3 mg/kg body weight, 5 mg/kg body weight or 10 mg/kg body weight or within the range of 1-10 mg/kg. An exemplary treatment regime entails administration once per week, once every two weeks, once every three weeks, once every four weeks, once a month, once every 3 months or once every three to 6 months. Preferred dosage regimens for an anti-B7-H4 antibody of this disclosure include 1 mg/kg body weight or 3 mg/kg body weight via intravenous administration, with the antibody being given using one of the following dosing schedules: (i) every four weeks for six dosages, then every three months; (ii) every three weeks; (iii) 3 mg/kg body weight once followed by 1 mg/kg body weight every three weeks.

In some methods, two or more monoclonal antibodies with different binding specificities are administered simultaneously, in which case the dosage of each antibody administered falls within the ranges indicated. Antibody is usually administered on multiple occasions. Intervals between single dosages can be, for example, weekly, monthly, every three months or yearly. Intervals can also be irregular as indicated by measuring blood levels of antibody to the target antigen in the patient. In some methods, dosage is adjusted to achieve a plasma antibody concentration of about 1-1000 μg/ml and in some methods about 25-300 μg/ml.

Alternatively, antibody can be administered as a sustained release formulation, in which case less frequent administration is required. Dosage and frequency vary depending on the half-life of the antibody in the patient. In general, human antibodies show the longest half life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

For use in the prophylaxis and/or treatment of diseases related to abnormal cellular proliferation, a circulating concentration of administered compound of about 0.001 μM to 20 μM is preferred, with about 0.01 μM to 5 μM being preferred.

Patient doses for oral administration of the compounds described herein, typically range from about 1 mg/day to about 10,000 mg/day, more typically from about 10 mg/day to about 1,000 mg/day, and most typically from about 50 mg/day to about 500 mg/day. Stated in terms of patient body weight, typical dosages range from about 0.01 to about 150 mg/kg/day, more typically from about 0.1 to about 15 mg/kg/day, and most typically from about 1 to about 10 mg/kg/day, for example 5 mg/kg/day or 3 mg/kg/day.

In at least some embodiments, patient doses that retard or inhibit tumor growth can be 1 μmol/kg/day or less. For example, the patient doses can be 0.9, 0.6, 0.5, 0.45, 0.3, 0.2, 0.15, or 0.1 μmol/kg/day or less (referring to moles of the drug). Preferably, the antibody-drug conjugate retards growth of the tumor when administered in the daily dosage amount over a period of at least five days. In at least some embodiments, the tumor is a human-type tumor in a SCID mouse. As an example, the SCID mouse can be a CB17.SCID mouse (available from Taconic, Germantown, N.Y.).

Actual dosage levels of the active ingredients in the pharmaceutical compositions of the present disclosure may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present disclosure employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A “therapeutically effective dosage” of an anti-B7-H4 antibody of this disclosure preferably results in a decrease in severity of disease symptoms, an increase in frequency and duration of disease symptom-free periods, or a prevention of impairment or disability due to the disease affliction. For example, for the treatment of tumor-bearing subjects, a “therapeutically effective dosage” preferably inhibits tumor growth by at least about 20%, more preferably by at least about 40%, even more preferably by at least about 60%, and still more preferably by at least about 80% relative to untreated subjects. The ability of a compound to inhibit tumor growth can be evaluated in an animal model system predictive of efficacy in human tumors. Alternatively, this property of a composition can be evaluated by examining the ability of the compound to inhibit cell growth, such inhibition can be measured in vitro by assays known to the skilled practitioner. A therapeutically effective amount of a therapeutic compound can decrease tumor size, or otherwise ameliorate symptoms in a subject. One of ordinary skill in the art would be able to determine such amounts based on such factors as the subject's size, the severity of the subject's symptoms, and the particular composition or route of administration selected.

A composition of the present disclosure can be administered via one or more routes of administration using one or more of a variety of methods known in the art. As will be appreciated by the skilled artisan, the route and/or mode of administration will vary depending upon the desired results. Preferred routes of administration for antibodies of this disclosure include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Alternatively, an antibody of this disclosure can be administered via a non-parenteral route, such as a topical, epidermal or mucosal route of administration, for example, intranasally, orally, vaginally, rectally, sublingually or topically.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Therapeutic compositions can be administered with medical devices known in the art. For example, in a preferred embodiment, a therapeutic composition of this disclosure can be administered with a needleless hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules useful in the present disclosure include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

In certain embodiments, the human monoclonal antibodies of this disclosure can be formulated to ensure proper distribution in vivo. For example, the blood-brain barrier (BBB) excludes many highly hydrophilic compounds. To ensure that the therapeutic compounds of this disclosure cross the BBB (if desired), they can be formulated, for example, in liposomes.

For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., V.V. Ranade (1989) J. Clin. Pharmacol. 29:685). Exemplary targeting moieties include folate or biotin (see, e.g., U.S. Pat. No. 5,416,016 to Low et al.); mannosides (Umezawa et al., (1988) Biochem. Biophys. Res. Commun. 153:1038); antibodies (P. G. Bloeman et al. (1995) FEBS Lett. 357:140; M. Owais et al. (1995) Antimicrob. Agents Chemother. 39:180); surfactant protein A receptor (Briscoe et al. (1995) Am. J. Physiol. 1233:134); p120 (Schreier et al. (1994) J. Biol. Chem. 269:9090); see also K. Keinanen; M. L. Laukkanen (1994) FEBS Lett. 346:123; J. J. Killion; I. J. Fidler (1994) Immunomethods 4:273.

Uses and Methods of this Disclosure

The antibody-partner molecule conjugates comprising antibodies, particularly the human antibodies, antibody compositions and methods of the present disclosure have numerous in vitro and in vivo diagnostic and therapeutic utilities involving, for example, detection of B7-H4, treatment of cancer or enhancement of immune response by blockade of B7-H4. In a preferred embodiment, the antibodies of the present disclosure are human antibodies. For example, these molecules can be administered to cells in culture, in vitro or ex vivo or to human subjects, e.g., in vivo, to treat, prevent and to diagnose a variety of disorders or to enhance immunity in a variety of situations.

As used herein, the term “subject” is intended to include human and non-human animals. The term “non-human animals” includes all vertebrates, e.g., mammals and non-mammals, such as non-human primates, sheep, dogs, cats, cows, horses, chickens, amphibians and reptiles. Preferred subjects include human patients having disorders associated with B7-H4 expression or in need of enhancement of an immune response. The methods are particularly suitable for treating human patients having a disorder associated with aberrant B7-H4 expression. The methods are also particularly suitable for treating human patients having a disorder that can be treated by augmenting the T-cell mediated immune response. To achieve antigen-specific enhancement of immunity, the anti-B7-H4 antibodies can be administered together with an antigen of interest. When antibodies to B7-H4 are administered together with another agent, the two can be administered in either order or simultaneously.

Given the specific binding of the antibodies of this disclosure for B7-H4, the antibodies of this disclosure can be used to specifically detect B7-H4 expression on the surface of cells and, moreover, can be used to purify B7-H4 via immunoaffinity purification.

B7-H4 is expressed in a variety of human cancers, including breast cell carcinomas, metastatic breast cancers, ovarian cell carcinomas, metastatic ovarian cancers and renal cell carcinomas (Tringler et al (2005) Clinical Cancer Res. U: 1842-48; Salceda et al. (2005) Exp Cell Res. 306:128-41; Tringler et al. (2006) Gynecol Oncol. 100:44-52; Krambeck et al. (2006) Proc Natl Acad Sci USA 103:10391-6; Chen et al. (2006) Kidney Int. Epub; Sun et al. (2006) Lung Cancer 53:143-51; Bignotti et al. (2006) Gynecol Oncol. 103:405-16; Kryczek et al. (2006) J Exp Med 203:871-81; Simon et al. (2006) Cancer Res. 66:1570-5). An anti-B7-H4 antibody may be used alone to inhibit the growth of cancerous tumors. Alternatively, an anti-B7-H4 antibody may be used in conjunction with other immunogenic agents, standard cancer treatments or other antibodies, as described below.

The B and T lymphocyte attenuator (BTLA) was found to be the receptor for B7-H4 and has an inhibitory effect on immune responses, similar to cytotoxic T lymphocyte antigen-4 (CTLA-4) and programmed death-1 (PD-1) (Carreno and Collins (2003) Trends Immunol 24:524-7). B7-H4 functions by negatively regulating T cell immunity by the inhibition of T-cell proliferation, cytokine production and cell cycle production (Choi et al. (2003) J Immunol. 171:4650-4). A B7-H4-Ig fusion protein inhibits T-cell activation, whereas blockade of B7-H4 by antibodies can enhance the immune response in the patient (Sica et al. (2003) Immunity 18:849-61).

In one aspect, the present disclosure relates to treatment of a subject in vivo using an anti-B7-H4 antibody such that growth of cancerous tumors is inhibited. An anti-B7-H4 antibody may be used alone to inhibit the growth of cancerous tumors. Alternatively, an anti-B7-H4 antibody may be used in conjunction with other immunogenic agents, standard cancer treatments or other antibodies, as described below.

Accordingly, in one embodiment, this disclosure provides a method of inhibiting growth of tumor cells in a subject, comprising administering to the subject a therapeutically effective amount of an anti-B7-H4 antibody or antigen-binding portion thereof. Preferably, the antibody is a human anti-B7-H4 antibody (such as any of the human anti-human B7-H4 antibodies described herein). Additionally or alternatively, the antibody may be a chimeric or humanized anti-B7-H4 antibody.

Preferred cancers whose growth may be inhibited using the antibodies of this disclosure include cancers typically responsive to immunotherapy. Non-limiting examples of preferred cancers for treatment include breast cancer (e.g., breast cell carcinoma), ovarian cancer (e.g., ovarian cell carcinoma) and renal cell carcinoma (RCC). Examples of other cancers that may be treated using the methods of this disclosure include melanoma (e.g., metastatic malignant melanoma), prostate cancer, colon cancer, lung cancer, bone cancer, pancreatic cancer, skin cancer, brain tumors, chronic or acute leukemias including acute myeloid leukemia, chronic myeloid leukemia, acute lymphoblastic leukemia, chronic lymphocytic leukemia, lymphomas (e.g., Hodgkin's and non-Hodgkin's lymphoma, lymphocytic lymphoma, primary CNS lymphoma, T-cell lymphoma) nasopharangeal carcinomas, cancer of the head or neck, cutaneous or intraocular malignant melanoma, uterine cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the breast gland, sarcoma of soft tissue, cancer of the urethra, cancer of the penis, solid tumors of childhood, cancer of the bladder, cancer of the kidney or ureter, carcinoma of the breast or pelvis, neoplasm of the central nervous system (CNS), tumor angiogenesis, spinal axis tumor, brain stem glioma, pituitary adenoma, Kaposi's sarcoma, epidermoid cancer, squamous cell cancer, environmentally induced cancers including those induced by asbestos, e.g., mesothelioma and combinations of said cancers.

Optionally, antibodies to B7-H4 can be combined with an immunogenic agent, such as cancerous cells, purified tumor antigens (including recombinant proteins, peptides and carbohydrate molecules), cells and cells transfected with genes encoding immune stimulating cytokines (He et al, J. Immunol. 173:4919-28 (2004)). Non-limiting examples of tumor vaccines that can be used include peptides of melanoma antigens, such as peptides of gp100, MAGE antigens, Trp-2, MARTl and/or tyrosinase or tumor cells transfected to express the cytokine GM-CSF. In humans, some tumors have been shown to be immunogenic such as melanomas. It is anticipated that by raising the threshold of T cell activation by B7-H4 blockade, tumors may be activated in responses in the host.

B7-H4 blockade is likely to be most effective when combined with a vaccination protocol. Many experimental strategies for vaccination against tumors have been devised (see, Rosenberg, “Development of Cancer Vaccines” ASCO Educational Book Spring: 60-62 (2000); Logothetis, ASCO Educational Book Spring: 300-302 (2000); Khayat, ASCO Educational Book Spring: 414-428 (2000); Foon, ASCO Educational Book Spring: 730-738 (2000); see also Restifo and Sznol, Cancer Vaccines, Ch. 61, pp. 3023-3043 in De Vita et al. (ed.) Cancer: Principles and Practice of Oncology, Fifth Edition (1997)). In one of these strategies, a vaccine is prepared using autologous or allogeneic tumor cells. Typically, these cellular vaccines are most effective when the tumor cells are transduced to express GM-CSF. GM-CSF has been shown to be a potent activator of antigen presentation for tumor vaccination (Dranoff et al. Proc. Natl. Acad. Sci. U.S.A. 90: 3539-43 (1993)).

The study of gene expression and large scale gene expression patterns in various tumors has led to the definition of so called tumor specific antigens (Rosenberg, Immunity 10:281-7 (1999)). In many cases, these tumor specific antigens are differentiation antigens expressed in the tumors and in the cell from which the tumor arose, for example melanocyte antigens gp100, MAGE antigens and Trp-2. More importantly, many of these antigens can be shown to be the targets of tumor specific T cells found in the host. B7-H4 blockade may be used in conjunction with a collection of recombinant proteins and/or peptides expressed in a tumor in order to generate an immune response to these proteins. These proteins are normally viewed by the immune system as self antigens and are therefore tolerant to them. The tumor antigen may also include the protein telomerase, which is required for the synthesis of telomeres of chromosomes and which is expressed in more than 85% of human cancers and in only a limited number of somatic tissues (Kim et al, Science 266:2011-2013 (1994)). (These somatic tissues may be protected from immune attack by various means). Tumor antigen may also be “neo-antigens” expressed in cancer cells because of somatic mutations that alter protein sequence or create fusion proteins between two unrelated sequences (i.e. bcr-abl in the Philadelphia chromosome) or idiotype from B cell tumors.

Other tumor vaccines may include the proteins from viruses implicated in human cancers such a Human Papilloma Viruses (HPV), Hepatitis Viruses (HBV and HCV) and Kaposi's Herpes Sarcoma Virus (KHSV). Another form of tumor specific antigen which may be used in conjunction with B7-H4 blockade is purified heat shock proteins (HSP) isolated from the tumor tissue itself. These heat shock proteins contain fragments of proteins from the tumor cells and these HSPs are highly efficient at delivery to antigen presenting cells for eliciting tumor immunity (Suot and Srivastava Science 269:1585-1588 (1995)); Tamura et al. Science 278:117-120 (1997)).

Dendritic cells (DC) are potent antigen presenting cells that can be used to prime antigen-specific responses. DCs can be produced ex vivo and loaded with various protein and peptide antigens as well as tumor cell extracts (Nestle, F. et al. (1998) Nature Medicine 4: 328-332). DCs may also be transduced by genetic means to express these tumor antigens as well. DCs have also been fused directly to tumor cells for the purposes of immunization (Kugler, A. et al. (2000) Nature Medicine 6:332-336). As a method of vaccination, DC immunization may be effectively combined with PD-1 blockade to activate more potent anti-tumor responses.

B7-H4 blockade may also be combined with standard cancer treatments. B7-H4 blockade may be effectively combined with chemotherapeutic regimes. In these instances, it may be possible to reduce the dose of chemotherapeutic reagent administered (Mokyr, M. et al. (1998) Cancer Research 58: 5301-5304). An example of such a combination is an anti-B7-H4 antibody in combination with decarbazine for the treatment of various cancers. Another example of such a combination is an anti-B7-H4 antibody in combination with interleukin-2 (IL-2) for the treatment of various cancers. The scientific rationale behind the combined use of B7-H4 blockade and chemotherapy is that cell death, that is a consequence of the cytotoxic action of most chemotherapeutic compounds, should result in increased levels of tumor antigen in the antigen presentation pathway. Other combination therapies that may result in synergy with B7-H4 blockade through cell death are radiation, surgery and hormone deprivation. Each of these protocols creates a source of tumor antigen in the host. Angiogenesis inhibitors may also be combined with B7-H4 blockade. Inhibition of angiogenesis leads to tumor cell death which may feed tumor antigen into host antigen presentation pathways.

B7-H4 blocking antibodies can also be used in combination with bispecific antibodies that target Fc alpha or Fc gamma receptor-expressing effectors cells to tumor cells (see, e.g., U.S. Pat. Nos. 5,922,845 and 5,837,243). Bispecific antibodies can be used to target two separate antigens. For example anti-Fc receptor/anti tumor antigen (e.g., Her-2/neu) bispecific antibodies have been used to target macrophages to sites of tumor. This targeting may more effectively activate tumor specific responses. The T cell arm of these responses would by augmented by the use of B7-H4 blockade. Alternatively, antigen may be delivered directly to DCs by the use of bispecific antibodies which bind to tumor antigen and a dendritic cell specific cell surface marker.

Tumors evade host immune surveillance by a large variety of mechanisms. Many of these mechanisms may be overcome by the inactivation of proteins which are expressed by the tumors and which are immunosuppressive. These include among others TGF-beta (Kehrl, J. et al (1986) J. Exp. Med. 163: 1037-1050), IL-10 (Howard, M. & O'Gara, A. (1992) Immunology Today 13: 198-200) and Fas ligand (Hahne, M. et al (1996) Science 274: 1363-1365). Antibodies to each of these entities may be used in combination with anti-PD-1 to counteract the effects of the immunosuppressive agent and favor tumor immune responses by the host.

Other antibodies which may be used to activate host immune responsiveness can be used in combination with anti-B7-H4. These include molecules on the surface of dendritic cells which activate DC function and antigen presentation. Anti-CD40 antibodies are able to substitute effectively for T cell helper activity (Ridge, J. et al. (1998) Nature 393: 474-478) and can be used in conjunction with B7-H4 antibodies. Activating antibodies to T cell costimulatory molecules such as CTLA-4 (e.g., U.S. Pat. No. 5,811,097), OX-40 (Weinberg, A. et al. (2000) Immunol 164: 2160-2169), 4-1BB (Melero, I. et al. (1997) Nature Medicine 3: 682-685 (1997), PD-1 (del Rio et al. (2005) Eur J Immunol 35:3545-60) and ICOS (Hutloff, A. et al (1999) Nature 397: 262-266) may also provide for increased levels of T cell activation.

Bone marrow transplantation is currently being used to treat a variety of tumors of hematopoietic origin. While graft versus host disease is a consequence of this treatment, therapeutic benefit may be obtained from graft vs. tumor responses. B7-H4 blockade can be used to increase the effectiveness of the donor engrafted tumor specific T cells.

There are also several experimental treatment protocols that involve ex vivo activation and expansion of antigen specific T cells and adoptive transfer of these cells into recipients in order to identify antigen-specific T cells against tumor (Greenberg, R. & Riddell, S. (1999) Science 285: 546-51). These methods may also be used to activate T cell responses to infectious agents such as CMV. Ex vivo activation in the presence of anti-B7-H4 antibodies may be expected to increase the frequency and activity of the adoptively transferred T cells.

Given the expression of B7-H4 on various tumor cells, the human antibodies, antibody compositions and methods of the present disclosure can be used to treat a subject with a tumorigenic disorder, e.g., a disorder characterized by the presence of tumor cells expressing B7-H4 including, for example, breast cancer (e.g., breast cell carcinoma), ovarian cancer (e.g., ovarian cell carcinoma), and renal cancer. Examples of other cancers that may be treated using the methods of the instant disclosure include melanoma (e.g., metastatic malignant melanoma), prostate cancer, colon cancer and lung cancer, bone cancer, pancreatic cancer, skin cancer, cancer of the head or neck, cutaneous or intraocular malignant melanoma, uterine cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, acute lymphocytic leukemia (ALL), chronic lymphocytic leukemia (CLL), Burkitt's lymphoma, anaplastic large-cell lymphomas (ALCL), multiple myeloma, cutaneous T-cell lymphomas, nodular small cleaved-cell lymphomas, lymphocytic lymphomas, peripheral T-cell lymphomas, Lennert's lymphomas, immunoblastic lymphomas, T-cell leukemia/lymphomas (ATLL), adult T-cell leukemia (T-ALL), entroblastic/centrocytic (cb/cc) follicular lymphomas cancers, diffuse large cell lymphomas of B lineage, angioimmunoblastic lymphadenopathy (AILD)-like T cell lymphoma, HIV associated body cavity based lymphomas, embryonal carcinomas, undifferentiated carcinomas of the rhino-pharynx (e.g., Schmincke's tumor), Castleman's disease, Kaposi's Sarcoma, multiple myeloma, Waldenstrom's macroglobulinemia and other B-cell lymphomas, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, glioblastoma, brain tumors, nasopharangeal carcinomas, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers. The present disclosure is also useful for treatment of metastatic cancers.

Accordingly, in one embodiment, this disclosure provides a method of inhibiting growth of tumor cells in a subject, comprising administering to the subject a therapeutically effective amount of an anti-B7-H4 antibody or antigen-binding portion thereof. Typically, the antibody is a human anti-B7-H4 antibody (such as any of the human anti-human B7-H4 antibodies described herein). Additionally or alternatively, the antibody may be a chimeric or humanized anti-B7-H4 antibody.

Other methods of this disclosure are used to treat patients that have been exposed to particular toxins or pathogens. Accordingly, another aspect of this disclosure provides a method of treating an infectious disease in a subject comprising administering to the subject an anti-B7-H4 antibody or antigen-binding portion thereof, such that the subject is treated for the infectious disease. Preferably, the antibody is a human anti-human B7-H4 antibody (such as any of the human anti-B7-H4 antibodies described herein). Additionally or alternatively, the antibody can be a chimeric or humanized antibody.

Similar to its application to tumors as discussed above, antibody mediated B7-H4 blockade can be used alone or as an adjuvant, in combination with vaccines, to stimulate the immune response to pathogens, toxins and self-antigens. Examples of pathogens for which this therapeutic approach may be particularly useful, include pathogens for which there is currently no effective vaccine or pathogens for which conventional vaccines are less than completely effective. These include, but are not limited to HIV, Hepatitis (A, B, & C), Influenza, Herpes, Giardia, Malaria, Leishmania, Staphylococcus aureus, Pseudomonas Aeruginosa. PD-1 blockade is particularly useful against established infections by agents such as HIV that present altered antigens over the course of the infections. These novel epitopes are recognized as foreign at the time of anti-human B7-H4 administration, thus provoking a strong T cell response that is not dampened by negative signals through B7-H4.

Some examples of pathogenic viruses causing infections treatable by methods of this disclosure include HIV, hepatitis (A, B or C), herpes virus (e.g., VZV, HSV-I, HAV-6, HSV-II and CMV, Epstein Barr virus), adenovirus, influenza virus, flaviviruses. echovirus, rhinovirus, coxsackie virus, cornovirus, respiratory syncytial virus, mumps virus, rotavirus, measles virus, rubella virus, parvovirus, vaccinia virus, HTLV virus, dengue virus, papilloma virus, molluscum virus, poliovirus, rabies virus, JC virus and arboviral encephalitis virus.

Some examples of pathogenic bacteria causing infections treatable by methods of this disclosure include chlamydia, rickettsial bacteria, mycobacteria, staphylococci, streptococci, pneumonococci, meningococci and conococci, klebsiella, proteus, serratia, pseudomonas, legionella, diphtheria, salmonella, bacilli, cholera, tetanus, botulism, anthrax, plague, leptospirosis and Lymes disease bacteria.

Some examples of pathogenic fungi causing infections treatable by methods of this disclosure include Candida (albicans, krusei, glabrata, tropicalis, etc.), Cryptococcus neoformans, Aspergillus (finnigatus, niger, etc.), Genus Mucorales (mucor, absidia, rhizophus), Sporothrix schenkii, Blastomyces dennatitidis, Paracoccidioides brasiliensis, Coccidioides immitis and Histoplasma capsulatum.

Some examples of pathogenic parasites causing infections treatable by methods of this disclosure include Entamoeba histolytica, Balantidium coli, Naegleriafowleri, Acanthamoeba sp., Giardia lambia, Cryptosporidium sp., Pneumocystis carinii, Plasmodium vivax, Babesia microti, Trypanosoma brucei, Trypanosoma cruzi, Leishmania donovani, Toxoplasma gondi, Nippostrongylus brasiliensis.

In all of the above methods, B7-H4 blockade can be combined with other forms of immunotherapy such as cytokine treatment (e.g., interferons, GM-CSF, G-CSF, IL-2) or bispecific antibody therapy, which provides for enhanced presentation of tumor antigens (see, e.g., Holliger (1993) Proc. Natl. Acad. Sci. USA 90:6444-6448; Poljak (1994) Structure 2:1121-1123).

Autoimmune reactions anti-B7-H4 antibodies may provoke and amplify autoimmune responses. Indeed, induction of anti-tumor responses using tumor cell and peptide vaccines reveals that many anti-tumor responses involve anti-self reactivities (depigmentation observed in anti-CTLA-4+GM-CSF-modified Bl 6 melanoma in van Elsas et al. supra; depigmentation in Trp-2 vaccinated mice (Overwijk, W. et al. (1999) Proc. Natl. Acad. Sci. U.S.A. 96: 2982-2987); autoimmune prostatitis evoked by TRAMP tumor cell vaccines (Hurwitz, A. (2000) supra), melanoma peptide antigen vaccination and vitilago observed in human clinical trials (Rosenberg, S A and White, D E (1996) J. Immunother Emphasis Tumor Immunol 19 (1): 81-4).

Therefore, it is possible to consider using anti-B7-H4 blockade in conjunction with various self proteins in order to devise vaccination protocols to efficiently generate immune responses against these self proteins for disease treatment. For example, Alzheimer's disease involves inappropriate accumulation of Aβ peptide in amyloid deposits in the brain; antibody responses against amyloid are able to clear these amyloid deposits (Schenk et al., (1999) Nature 400: 173-177).

Other self proteins may also be used as targets such as IgE for the treatment of allergy and asthma and TNFα for rheumatoid arthritis. Finally, antibody responses to various hormones may be induced by the use of anti-B7-H4 antibody. Neutralizing antibody responses to reproductive hormones may be used for contraception. Neutralizing antibody response to hormones and other soluble factors that are required for the growth of particular tumors may also be considered as possible vaccination targets. Analogous methods as described above for the use of anti-B7-H4 antibody can be used for induction of therapeutic autoimmune responses to treat patients having an inappropriate accumulation of other self-antigens, such as amyloid deposits, including Aβ in Alzheimer's disease, cytokines such as TNFα and IgE. Vaccines Anti-B7-H4 antibodies may be used to stimulate antigen-specific immune responses by coadministration of an anti-B7-H4 antibody with an antigen of interest (e.g., a vaccine). Accordingly, in another aspect this disclosure provides a method of enhancing an immune response to an antigen in a subject, comprising administering to the subject: (i) the antigen; and (ii) an anti-B7-H4 antibody or antigen-binding portion thereof, such that an immune response to the antigen in the subject is enhanced. Preferably, the antibody is a human anti-human B7-H4 antibody (such as any of the human anti-B7-H4 antibodies described herein). Additionally or alternatively, the antibody can be a chimeric or humanized antibody. The antigen can be, for example, a tumor antigen, a viral antigen, a bacterial antigen or an antigen from a pathogen. Non-limiting examples of such antigens include those discussed in the sections above, such as the tumor antigens (or tumor vaccines) discussed above or antigens from the viruses, bacteria or other pathogens described above.

Suitable routes of administering the antibody compositions (e.g., human monoclonal antibodies, multispecific and bispecific molecules and immunoconjugates) of this disclosure in vivo and in vitro are well known in the art and can be selected by those of ordinary skill. For example, the antibody compositions can be administered by injection (e.g., intravenous or subcutaneous). Suitable dosages of the molecules used will depend on the age and weight of the subject and the concentration and/or formulation of the antibody composition.

As previously described, human anti-B7-H4 antibodies of this disclosure can be coadministered with one or other more therapeutic agents, e.g., a cytotoxic agent, a radiotoxic agent or an immunosuppressive agent. The antibody can be linked to the agent (as an immunocomplex) or can be administered separate from the agent. In the latter case (separate administration), the antibody can be administered before, after or concurrently with the agent or can be co-administered with other known therapies, e.g., an anti-cancer therapy, e.g., radiation. Such therapeutic agents include, among others, antineoplastic agents such as doxorubicin (adriamycin), cisplatin bleomycin sulfate, carmustine, chlorambucil, decarbazine and cyclophosphamide hydroxyurea which, by themselves, are only effective at levels which are toxic or subtoxic to a patient. Cisplatin is intravenously administered as a 100 mg/dose once every four weeks and adriamycin is intravenously administered as a 60-75 mg/ml dose once every 21 days. Co-administration of the human anti-B7-H4 antibodies or antigen binding fragments thereof, of the present disclosure with chemotherapeutic agents provides two anticancer agents which operate via different mechanisms which yield a cytotoxic effect to human tumor cells. Such co-administration can solve problems due to development of resistance to drugs or a change in the antigenicity of the tumor cells which would render them unreactive with the antibody. Also within the scope of the present disclosure are kits comprising the antibody compositions of this disclosure (e.g., human antibodies, bispecific or multispecific molecules or immunoconjugates) and instructions for use. The kit can further contain a least one additional reagent or one or more additional human antibodies of this disclosure (e.g., a human antibody having a complementary activity which binds to an epitope in B7-H4 antigen distinct from the first human antibody). Kits typically include a label indicating the intended use of the contents of the kit. The term label includes any writing or recorded material supplied on or with the kit or which otherwise accompanies the kit.

In one embodiment, the present disclosure provides a method for treating a hyperproliferative disease, comprising administering an B7-H4 antibody and a CTLA-4 and/or PD-1 antibody to a subject. In further embodiments, the anti-B7-H4 antibody is administered at a subtherapeutic dose, the anti-CTLA-4 and/or PD-1 antibody is administered at a subtherapeutic dose or both are administered at a subtherapeutic dose. In another embodiment, the present disclosure provides a method for altering an adverse event associated with treatment of a hyperproliferative disease with an immunostimulatory agent, comprising administering an anti-B7-H4 antibody and a subtherapeutic dose of anti-CTLA-4 and/or anti-PD-1 antibody to a subject.

In certain embodiments, the subject is human. In certain embodiments, the anti-CTLA-4 antibody is human sequence monoclonal antibody 10D1 and the anti-PD-1 antibody is human sequence monoclonal antibody, such as 17D8, 2D3, 4H1, 5C4 and 4Al1. Human sequence monoclonal antibody 10D1 has been isolated and structurally characterized, as described in U.S. Pat. No. 6,984,720. Human sequence monoclonal antibodies 17D8, 2D3, 4H1, 5C4 and 4Al1 have been isolated and structurally characterized, as described in U.S. Provisional Patent No. 60/679,466.

The anti-B7-H4, anti-CTLA-4 antibody and anti-PD-1 monoclonal antibodies (mAbs) and the human sequence antibodies of this disclosure can be produced by a variety of techniques, including conventional monoclonal antibody methodology, e.g., the standard somatic cell hybridization technique of Kohler and Milstein (1975) Nature 256:495. Any technique for producing monoclonal antibody can be employed, e.g., viral or oncogenic transformation of B lymphocytes. One animal system for preparing hybridomas is the murine system. Hybridoma production in the mouse is a very well-established procedure. Immunization protocols and techniques for isolation of immunized splenocytes for fusion are known in the art. Fusion partners {e.g., murine myeloma cells) and fusion procedures are also known (see, e.g., Harlow and Lane (1988) Antibodies, A Laboratory Manual, Cold Spring Harbor Laboratory Press, Cold Spring Harbor N.Y.). The combination of antibodies is useful for enhancement of an immune response against a hyperproliferative disease by blockade of B7-H4 and PD-1 and/or CTLA-4. In a preferred embodiment, the antibodies of the present disclosure are human antibodies. For example, these molecules can be administered to cells in culture, in vitro or ex vivo or to human subjects, e.g., in vivo, to enhance immunity in a variety of situations. Accordingly, in one aspect, this disclosure provides a method of modifying an immune response in a subject comprising administering to the subject an antibody combination or a combination of antigen-binding portions thereof, of this disclosure such that the immune response in the subject is modified. Preferably, the response is enhanced, stimulated or up-regulated. In another embodiment, the instant disclosure provides a method of altering adverse events associated with treatment of a hyperproliferative disease with an immunostimulatory therapeutic agent, comprising administering an anti-B7-H4 antibody and a subtherapeutic dose of anti-CTLA-4 or anti-PD-1 antibody to a subject.

Blockade of B7-H4, PD-1 and CTLA-4 by antibodies can enhance the immune response to cancerous cells in the patient. Cancers whose growth may be inhibited using the antibodies of the instant disclosure include cancers typically responsive to immunotherapy. Representative examples of cancers for treatment with the combination therapy of the instant disclosure include melanoma (e.g., metastatic malignant melanoma), renal cancer, prostate cancer, breast cancer, colon cancer and lung cancer. Examples of other cancers that may be treated using the methods of the instant disclosure include bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS) 5 primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos and combinations of said cancers. The present disclosure is also useful for treatment of metastatic cancers.

In certain embodiments, the combination of therapeutic antibodies discussed herein may be administered concurrently as a single composition in a pharmaceutically acceptable carrier or concurrently as separate compositions with each antibody in a pharmaceutically acceptable carrier. In another embodiment, the combination of therapeutic antibodies can be administered sequentially. For example, an anti-B7-H4 antibody and an anti-PD-1 antibody can be administered sequentially, such as anti-B7-H4 being administered first and anti-PD-1 second or anti-PD-1 being administered first and anti-B7-H4 second. Furthermore, if more than one dose of the combination therapy is administered sequentially, the order of the sequential administration can be reversed or kept in the same order at each time point of administration, sequential administrations may be combined with concurrent administrations or any combination thereof. For example, the first administration of a combination anti-B7-H4 antibody and anti-PD-1 antibody may be concurrent, the second administration may be sequential with anti-B7-H4 first and anti-PD-1 second and the third administration may be sequential with anti-PD-1 first and anti-B7-H4 second, etc. Another representative dosing scheme may involve a first administration that is sequential with anti-PD-1 first and anti-B7-H4 second and subsequent administrations may be concurrent.

Optionally, the combination of anti-B7-H4 and anti-CTLA-4 and/or anti-PD-1 antibodies can be further combined with an immunogenic agent, such as cancerous cells, purified tumor antigens (including recombinant proteins, peptides and carbohydrate molecules), cells and cells transfected with genes encoding immune stimulating cytokines (He et al. (2004) J. Immunol. 173:4919-28). Non-limiting examples of tumor vaccines that can be used include peptides of melanoma antigens, such as peptides of gp100, MAGE antigens, Trp-2, MARTl and/or tyrosinase or tumor cells transfected to express the cytokine GM-CSF (discussed further below).

A combined B7-H4 and PD-1 and/or CTL A-4 blockade can be further combined with a vaccination protocol. Many experimental strategies for vaccination against tumors have been devised (see Rosenberg, S. (2000) Development of Cancer Vaccines, ASCO Educational Book Spring: 60-62; Logothetis, C, 2000, ASCO Educational Book Spring: 300-302; Khayat, D. (2000) ASCO Educational Book Spring: 414-428; Foon, K. (2000) ASCO Educational Book Spring: 730-738; see also Restifo and Sznol, Cancer Vaccines, Ch. 61, pp. 3023-3043 in De Vita et al (eds.), 1997, Cancer: Principles and Practice of Oncology. Fifth Edition). In one of these strategies, a vaccine is prepared using autologous or allogeneic tumor cells. These cellular vaccines have been shown to be most effective when the tumor cells are transduced to express GM-CSF. GM-CSF has been shown to be a potent activator of antigen presentation for tumor vaccination (Dranoff et al. (1993) Proc. Natl. Acad. Sci. U.S.A. 90: 3539-43).

A combined B7-H4 and PD-1 and/or CTLA-4 blockade may also be further combined with Standard cancer treatments. For example, a combined B7-H4 and PD-1 and/or CTLA-4 blockade may be effectively combined with chemotherapeutic regimes. In these instances, as is observed with the combination of anti-B7-H4 and anti-CTLA-4 and/or anti-PD-1 antibodies, it may be possible to reduce the dose of other chemotherapeutic reagent administered with the combination of the instant disclosure (Mokyr et al. (1998) Cancer Research 58: 5301-5304). The scientific rationale behind the combined use of B7-H4 and PD-1 and/or CTLA-4 blockade with chemotherapy is that cell death, which is a consequence of the cytotoxic action of most chemotherapeutic compounds, should result in increased levels of tumor antigen in the antigen presentation pathway. Other combination therapies that may result in synergy with a combined B7-H4 and PD-1 and/or CTLA-4 blockade through cell death include radiation, surgery or hormone deprivation. Each of these protocols creates a source of tumor antigen in the host. Angiogenesis inhibitors may also be combined with a combined B7-H4 and PD-1 and/or CTLA-4 blockade Inhibition of angiogenesis leads to tumor cell death, which may also be a source of tumor antigen to be fed into host antigen presentation pathways.

A combination of B7-H4 and PD-1 and/or CTLA-4 blocking antibodies can also be used in combination with bi specific antibodies that target Fcα or Fcγ receptor-expressing effector cells to tumor cells (see, e.g., U.S. Pat. Nos. 5,922,845 and 5,837,243). Bispecific antibodies can be used to target two separate antigens. For example anti-Fc receptor/anti tumor antigen (e.g., Her-2/neu) bispecific antibodies have been used to target macrophages to sites of tumor. This targeting may more effectively activate tumor specific responses. The T cell arm of these responses would by augmented by the use of a combined B7-H4 and PD-1 and/or CTLA-4 blockade. Alternatively, antigen may be delivered directly to DCs by the use of bispecific antibodies which bind to tumor antigen and a dendritic cell specific cell surface marker. In another example, a combination of anti-PD-1 and anti-CTLA-4 antibodies can be used in conjunction with anti-neoplastic antibodies, such as Riruxan® (rituximab), Herceptin® (trastuzumab), Bexxar® (tositumomab), Zevalin® (ibritumomab), Campath® (alemtuzumab), Lymphocide® (eprtuzumab), Avastin® (bevacizumab) and Tarceva® (erlotinib) and the like. By way of example and not wishing to be bound by theory, treatment with an anti-cancer antibody or an anti-cancer antibody conjugated to a toxin can lead to cancer cell death tumor cells) which would potentiate an immune response mediated by B7-H4, CTLA-4 or PD-1. In an exemplary embodiment, a treatment of a hyperproliferative disease (e.g., a cancer tumor) may include an anti-cancer antibody in combination with anti-B7-H4 and anti-PD-1 and/or anti-CTLA-4 antibodies, concurrently or sequentially or any combination thereof, which may potentiate an anti-tumor immune responses by the host. Tumors evade host immune surveillance by a large variety of mechanisms. Many of these mechanisms may be overcome by the inactivation of proteins, which are expressed by the tumors and which are immunosuppressive. These include, among others, TGF-β (Kehrl, J. et al. (1986) J Exp. Med. 163: 1037-1050), IL-10 (Howard, M. & O'Garra, A. (1992) Immunology Today 13: 198-200) and Fas ligand (Hahne, M. et al. (1996) Science 274: 1363-1365). In another example, antibodies to each of these entities may be further combined with an anti-B7-H4 and anti-PD-1 and/or anti-CTLA-4 combination to counteract the effects of immunosuppressive agents and favor anti-tumor immune responses by the host.

Other antibodies that may be used to activate host immune responsiveness can be further used in combination with an anti-B7-H4 and anti-PD-1 and/or anti-CTLA-4 combination. These include molecules on the surface of dendritic cells that activate DC function and antigen presentation. Anti-CD40 antibodies are able to substitute effectively for T cell helper activity (Ridge, J. et al. (1998) Nature 393: 474-478) and have been shown efficacious in conjunction with anti-CTLA-4 (Ito, N. et al. (2000) Immunobiology 201 (5) 527-40). Activating antibodies to T cell costimulatory molecules, such as OX-40 (Weinberg, A. et al. (2000) Immunol 164: 2160-2169), 4-1BB (Melero, I. et al. (1997) Nature Medicine 3: 682-685 (1997), PD-1 (del Rio et al. (2005) Eur J Immunol. 35:3545-60) and ICOS (Hutloff, A. et al. (1999) Nature 397: 262-266) may also provide for increased levels of T cell activation.

Bone marrow transplantation is currently being used to treat a variety of tumors of hematopoietic origin. While graft versus host disease is a consequence of this treatment, therapeutic benefit may be obtained from graft vs. tumor responses. A combined B7-H4 and PD-1 and/or CTLA-4 blockade can be used to increase the effectiveness of the donor engrafted tumor specific T cells.

There are also several experimental treatment protocols that involve ex vivo activation and expansion of antigen specific T cells and adoptive transfer of these cells into recipients in order to antigen-specific T cells against tumor (Greenberg, R. & Riddell, S. (1999) Science 285:546-51). These methods may also be used to activate T cell responses to infectious agents such as CMV. Ex vivo activation in the presence of anti-B7-H4 and anti-PD-1 and/or anti-CTLA-4 antibodies may be expected to increase the frequency and activity of the adoptively transferred T cells.

As set forth herein organs can exhibit immune-related adverse events following immunostimulatory therapeutic antibody therapy, such as the GI tract (diarrhea and colitis) and the skin (rash and pruritis) after treatment with anti-CTLA-4 antibody. For example, non-colonic gastrointestinal immune-related adverse events have also been observed in the esophagus (esophagitis), duodenum (duodenitis) and ileum (ileitis) after anti-CTLA-4 antibody treatment.

In certain embodiments, the present disclosure provides a method for altering an adverse event associated with treatment of a hyperproliferative disease with an immunostimulatory agent, comprising administering a anti-B7-H4 antibody and a subtherapeutic dose of anti-CTLA-4 antibody to a subject. For example, the methods of the present disclosure provide for a method of reducing the incidence of immunostimulatory therapeutic antibody-induced colitis or diarrhea by administering a non-absorbable steroid to the patient. Because any patient who will receive an immunostimulatory therapeutic antibody is at risk for developing colitis or diarrhea induced by such an antibody, this entire patient population is suitable for therapy according to the methods of the present disclosure. Although steroids have been administered to treat inflammatory bowel disease (IBD) and prevent exacerbations of IBD, they have not been used to prevent (decrease the incidence of) IBD in patients who have not been diagnosed with IBD. The significant side effects associated with steroids, even non-absorbable steroids, have discouraged prophylactic use.

In further embodiments, a combination B7-H4 and PD-1 and/or CTLA-4 blockade (i.e., immunostimulatory therapeutic antibodies anti-B7-H4 and anti-PD-1 and/or anti-CTLA-4) can be further combined with the use of any non-absorbable steroid. As used herein, a “non-absorbable steroid” is a glucocorticoid that exhibits extensive first pass metabolism such that, following metabolism in the liver, the bioavailability of the steroid is low, i.e., less than about 20%. In one embodiment of this disclosure, the non-absorbable steroid is budesonide. Budesonide is a locally-acting glucocorticosteroid, which is extensively metabolized, primarily by the liver, following oral administration. ENTOCORT EC® (Astra-Zeneca) is a pH- and time-dependent oral formulation of budesonide developed to optimize drug delivery to the ileum and throughout the colon. ENTOCORT EC® is approved in the U.S. for the treatment of mild to moderate Crohn's disease involving the ileum and/or ascending colon. The usual oral dosage of ENTOCORT EC® for the treatment of Crohn's disease is 6 to 9 mg/day. ENTOCORT EC® is released in the intestines before being absorbed and retained in the gut mucosa. Once it passes through the gut mucosa target tissue, ENTOCORT EC® is extensively metabolized by the cytochrome P450 system in the liver to metabolites with negligible glucocorticoid activity. Therefore, the bioavailability is low (about 10%). The low bioavailability of budesonide results in an improved therapeutic ratio compared to other glucocorticoids with less extensive first-pass metabolism. Budesonide results in fewer adverse effects, including less hypothalamic-pituitary suppression, than systemically-acting corticosteroids. However, chronic administration of ENTOCORT ECO can result in systemic glucocorticoid effects such as hypercorticism and adrenal suppression. See PDR 58th ed. 2004; 608-610.

In still further embodiments, a combination B7-H4 and PD-1 and/or CTL A-4 blockade (i.e., immunostimulatory therapeutic antibodies anti-B7-H4 and anti-PD-1 and/or anti-CTLA-4) in conjunction with a non-absorbable steroid can be further combined with a salicylate. Salicylates include 5-ASA agents such as, for example: sulfasalazine (AZULFIDINE®, Pharmacia & UpJohn); olsalazine (DIPENTUM®, Pharmacia & UpJohn); balsalazide (COLAZAL®, Salix Pharmaceuticals, Inc.); and mesalamine (ASACOL®, Procter & Gamble Pharmaceuticals; PENTASA®, Shire US; CANASA®, Axcan Scandipharm, Inc.; ROWASA®, Solvay). In accordance with the methods of the present disclosure, a salicylate administered in combination with anti-B7-H4 and anti-PD-1 and/or anti-CTLA-4 antibodies and a non-absorbable steroid can includes any overlapping or sequential administration of the salicylate and the nonabsorbable steroid for the purpose of decreasing the incidence of colitis induced by the immunostimulatory antibodies. Thus, for example, methods for reducing the incidence of colitis induced by the immunostimulatory antibodies according to the present disclosure encompass administering a salicylate and a non-absorbable steroid concurrently or sequentially (e.g., a salicylate is administered 6 hours after a non-absorbable steroid) or any combination thereof. Further, according to the present disclosure, a salicylate and a non-absorbable steroid can be administered by the same route (e.g., both are administered orally) or by different routes (e.g., a salicylate is administered orally and a non-absorbable steroid is administered rectally), which may differ from the route(s) used to administer the anti-B7-H4, anti-PD-1 and anti-CTLA-4 antibodies.

The compositions (e.g., human antibodies, multispecific and bispecific molecules and immunoconjugates) of this disclosure which have complement binding sites, such as portions from IgG1, -2 or -3 or IgM which bind complement, can also be used in the presence of complement. In one embodiment, ex vivo treatment of a population of cells comprising target cells with a binding agent of this disclosure and appropriate effector cells can be supplemented by the addition of complement or serum containing complement. Phagocytosis of target cells coated with a binding agent of this disclosure can be improved by binding of complement proteins. In another embodiment target cells coated with the compositions (e.g., human antibodies, multispecific and bispecific molecules) of this disclosure can also be lysed by complement. In yet another embodiment, the compositions of this disclosure do not activate complement.

The compositions (e.g., human antibodies, multispecific and bispecific molecules and immunoconjugates) of this disclosure can also be administered together with complement. Accordingly, within the scope of this disclosure are compositions comprising human antibodies, multispecific or bispecific molecules and serum or complement. These compositions are advantageous in that the complement is located in close proximity to the human antibodies, multispecific or bispecific molecules. Alternatively, the human antibodies, multispecific or bispecific molecules of this disclosure and the complement or serum can be administered separately. Accordingly, patients treated with antibody compositions of this disclosure can be additionally administered (prior to, simultaneously with or following administration of a human antibody of this disclosure) with another therapeutic agent, such as a cytotoxic or radiotoxic agent, which enhances or augments the therapeutic effect of the human antibodies.

In other embodiments, the subject can be additionally treated with an agent that modulates, e.g., enhances or inhibits, the expression or activity of Fcγ or Fcγ receptors by, for example, treating the subject with a cytokine. Preferred cytokines for administration during treatment with the multispecific molecule include of granulocyte colony-stimulating factor (G-CSF), granulocyte-macrophage colony-stimulating factor (GM-CSF), interferon-γ (LEN-γ) and tumor necrosis factor (TNF).

The compositions (e.g., human antibodies, multispecific and bispecific molecules) of this disclosure can also be used to target cells expressing FcγR or B7-H4, for example for labeling such cells. For such use, the binding agent can be linked to a molecule that can be detected. Thus, this disclosure provides methods for localizing ex vivo or in vitro cells expressing Fc receptors, such as FcγR or B7-H4. The detectable label can be, e.g., a radioisotope, a fluorescent compound, an enzyme or an enzyme co-factor.

In a particular embodiment, this disclosure provides methods for detecting the presence of B7-H4 antigen in a sample or measuring the amount of B7-H4 antigen, comprising contacting the sample and a control sample, with a human monoclonal antibody or an antigen binding portion thereof, which specifically binds to B7-H4, under conditions that allow for formation of a complex between the antibody or portion thereof and B7-H4. The formation of a complex is then detected, wherein a difference complex formation between the sample compared to the control sample is indicative the presence of B7-H4 antigen in the sample.

In other embodiments, this disclosure provides methods for treating a B7-H4 mediated disorder in a subject.

In yet another embodiment, antibody-partner molecule conjugates of this disclosure can be used to target compounds (e.g., therapeutic agents, labels, cytotoxins, radiotoxoins immunosuppressants, etc.) to cells which have B7-H4 cell surface receptors by linking such compounds to the antibody. For example, an anti-B7-H4 antibody can be conjugated to UPT5 as described in U.S. patent application Ser. Nos. 10/160,972, 10/161,233, 10/161,234, 11/134,826, 11/134,685 and U.S. Provisional Patent Application No. 60/720,499 and/or any of the toxin compounds described in U.S. Pat. Nos. 6,281,354 and 6,548,530, US patent publication Nos. 20030050331, 20030064984, 20030073852 and 20040087497 or published in WO 03/022806, which are hereby incorporated by reference in their entireties. Thus, this disclosure also provides methods for localizing ex vivo or in vivo cells expressing B7-H4 (e.g., with a detectable label, such as a radioisotope, a fluorescent compound, an enzyme or an enzyme co-factor). Alternatively, the antibody-partner molecule conjugates can be used to kill cells which have B7-H4 cell surface receptors by targeting cytotoxins or radiotoxins to B7-H4.

The present disclosure is further illustrated by the following examples which should not be construed as further limiting. The contents of all figures and all references, patents and published patent applications cited throughout this application are expressly incorporated herein by reference.

### EXAMPLES

### Example 1

### Generation of Human Monoclonal Antibodies Against O8E

This Example discloses the generation of human monoclonal antibodies that specifically bind to human O8E (a/k/a B7H4, B7S1 and B7x).

### Antigen

CHO and HEK-293 cells were transfected with O8E using standard recombinant transfection methods and used as antigen for immunization. In addition, recombinant O8E alone was also used as antigen for immunization.

### Transgenic HuMAb Mouse® and KM Mouse®

Fully human monoclonal antibodies to O8E were prepared using the HCo7 and HCo12 strains of the transgenic HuMAb Mouse® and the KM strain of transgenic transchromosomic mice, each of which express human antibody genes. In each of these mouse strains, the endogenous mouse kappa light chain gene has been homozygously disrupted as described in Chen et al. (1993) EMBO J. 12:811-820 and the endogenous mouse heavy chain gene has been homozygously disrupted as described in Example 1 of PCT Publication WO 01/09187. Each of these mouse strains carries a human kappa light chain transgene, KCo5, as described in Fishwild et al. (1996) Nature Biotechnology 14:845-851. The HCo7 strain carries the HCo7 human heavy chain transgene as described in U.S. Pat. Nos. 5,545,806; 5,625,825; and, 5,545,807. The HCo12 strain carries the HCo12 human heavy chain transgene as described in Example 2 of PCT Publication WO 01/09187. The KM Mouse® strain contains the SC20 transchromosome as described in PCT Publication WO 02/43478.

### HuMAb and KM Immunizations:

To generate fully human monoclonal antibodies to O8E, mice of the HuMAb Mouse° and KM Mouse° were immunized with CHO-O8E transfected cells, HEK293-O8E transfected cells and/or purified recombinant O8E protein. General immunization schemes for HuMAb Mouse® are described in Lonberg, N. et al (1994) Nature 368(6474): 856-859; Fishwild, D. et al. (1996) Nature Biotechnology 14: 845-851 and PCT Publication WO 98/24884. The mice were 6-16 weeks of age upon the first infusion of antigen. A purified recombinant preparation (5-50 μg) of O8E protein was used to immunize the HuMAb Mice™ and KM Mice™.

Transgenic mice were immunized twice with antigen in complete Freund's adjuvant adjuvant either intraperitonealy (IP) or subcutaneously (Sc), followed by 3-21 days IP or SC immunization (up to a total of 11 immunizations) with the antigen in incomplete Freund's adjuvant. The immune response was monitored by retroorbital bleeds. The plasma was screened by ELISA (as described below) and mice with sufficient titers of anti-O8E human immunoglobulin were used for fusions. Mice were boosted intravenously with antigen 3 and 2 days before sacrifice and removal of the spleen. Typically, 10-35 fusions for each antigen were performed. Several dozen mice were immunized for each antigen.

### Selection of HuMb Mice™ or KM Mice™ Producing Anti-O8E Antibodies:

To select HuMab Mice™ or KM Mice™ producing antibodies that bound O8E sera from immunized mice was tested by ELISA as described by Fishwild, D. et al. (1996) (supra). Briefly, microtiter plates were coated with purified recombinant O8E at 1-2 μg/ml in PBS, 50 μl/wells incubated 4° C. overnight then blocked with 200 μl/well of 5% chicken serum in PBS/Tween (0.05%). Dilutions of plasma from O8E-immunized mice were added to each well and incubated for 1-2 hours at ambient temperature. The plates were washed with PBS/Tween and then incubated with a goat-anti-human IgG Fc polyclonal antibody conjugated with horseradish peroxidase (HRP) for 1 hour at room temperature. After washing, the plates were developed with ABTS substrate (Sigma, A-1888, 0.22 mg/ml) and analyzed by spectrophotometer at OD 415-495. Mice that developed the highest titers of anti-O8E antibodies were used for fusions. Fusions were performed as described below and hybridoma supernatants were tested for anti-O8E activity by ELISA and FACS.

### Generation of Hybridomas Producing Human Monoclonal Antibodies to O8E:

The mouse splenocytes, isolated from the HuMab Mice™ and KM Mice™, were fused with PEG to a mouse myeloma cell line either using PEG based upon standard protocols. The resulting hybridomas were then screened for the production of antigen-specific antibodies. Single cell suspensions of splenic lymphocytes from immunized mice were fused to one-fourth the number of SP2/0 nonsecreting mouse myeloma cells (ATCC, CRL 1581) with 50% PEG (Sigma). Cells were plated at approximately 1×10⁵ cells/well in flat bottom microliter plate, followed by a about two week incubation in selective medium containing 10% fetal bovine serum (Hyclone, Logan, Utah), 10% P388DI (ATCC, CRL TIB-63) conditioned medium, 3-5% origen (IGEN) in DMEM (Mediatech, CRL 10013, with high glucose, L-glutamine and sodium pyruvate) plus 5 mM HEPES, 0.055 mM 2-mercaptoethanol, 50 mg/ml gentamycin and 1×HAT (Sigma, CRL P-7185). After one to two weeks, cells were cultured in medium in which HAT was replaced with HT. Individual wells were then screened by ELISA and FACS (described above) for human anti-O8E monoclonal IgG antibodies. The positive clones were then screened for O8E positive antibodies on O8E recombinant protein by ELISA or on O8E expressing cells, for example CHO-O8E transfected cells, by FACS. Briefly, O8E-expressing cells were freshly harvested from tissue culture flasks and a single cell suspension prepared. O8E-expressing cell suspensions were either stained with primary antibody directly or after fixation with 1% paraformaldehyde in PBS. Approximately one million cells were resuspended in PBS containing 0.5% BSA and 50-200 μg/ml of primary antibody and incubated on ice for 30 minutes. The cells were washed twice with PBS containing 0.1% BSA, 0.01% NaN₃, resuspended in 100 μl of 1:100 diluted FITC-conjugated goat-anti-human IgG (Jackson ImmunoResearch, West Grove, Pa.) and incubated on ice for an additional 30 minutes. The cells were again washed twice, resuspended in 0.5 ml of wash buffer and analyzed for fluorescent staining on a FACSCalibur cytometer (Becton-Dickinson, San Jose, Calif.).

Once extensive hybridoma growth occurred, medium was monitored usually after 10-14 days. The antibody-secreting hybridomas were replated, screened again and, if still positive for human IgG, anti-O8E monoclonal antibodies were subcloned at least twice by limiting dilution. The stable subclones were then cultured in vitro to generate small amounts of antibody in tissue culture medium for further characterization.

Hybridoma clones 1G11, 2A7, 2F9, 12E1 and 13D12 were selected for further analysis.

### Example 2

### Structural Characterization of Human Monoclonal Antibodies 1G11, 2A 7, 2F9, 12E1 and 13D12

This Example discloses sequence analysis five (5) human monoclonal antibodies that specifically bind to O8E.

The cDNA sequences encoding the heavy and light chain variable regions of the 1G11, 2A7, 2F9, 12E1 and 13D12 monoclonal antibodies were obtained from the 1G11, 2A7, 2F9, 12E1 and 13D12 hybridomas, respectively, using standard PCR techniques and were sequenced using standard DNA sequencing techniques.

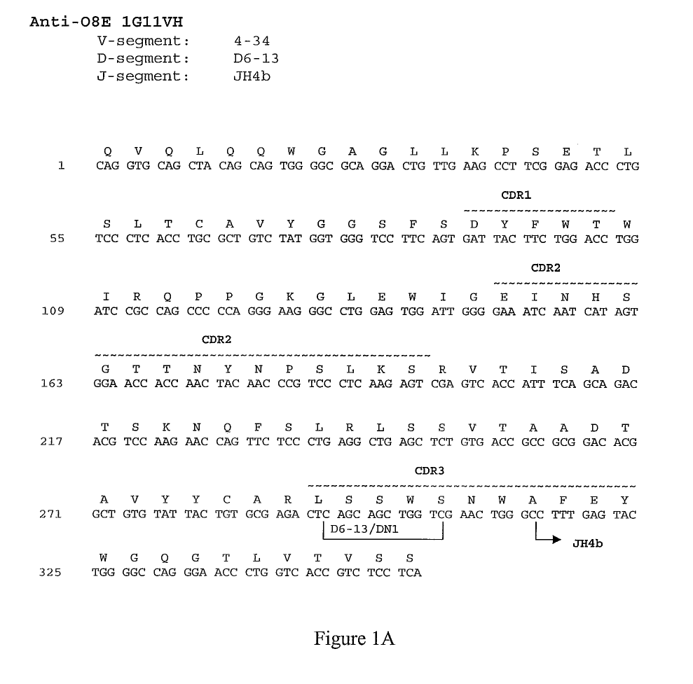

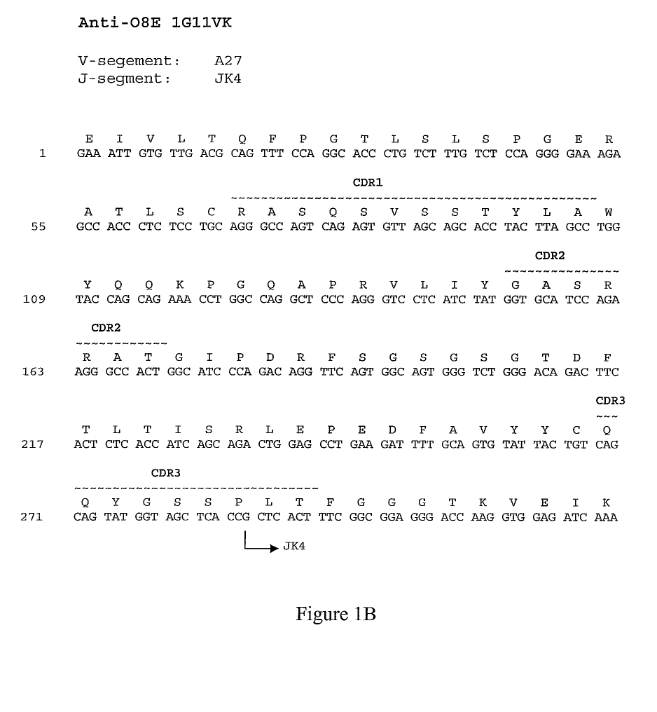

The nucleotide and amino acid sequences of the heavy chain variable region of 1G11 are shown in FIG. 1A and in SEQ ID NOs: 41 and 1, respectively.

The nucleotide and amino acid sequences of the light chain variable region of 1G11 are shown in FIG. 1B and in SEQ ID NO: 46 and 6, respectively.

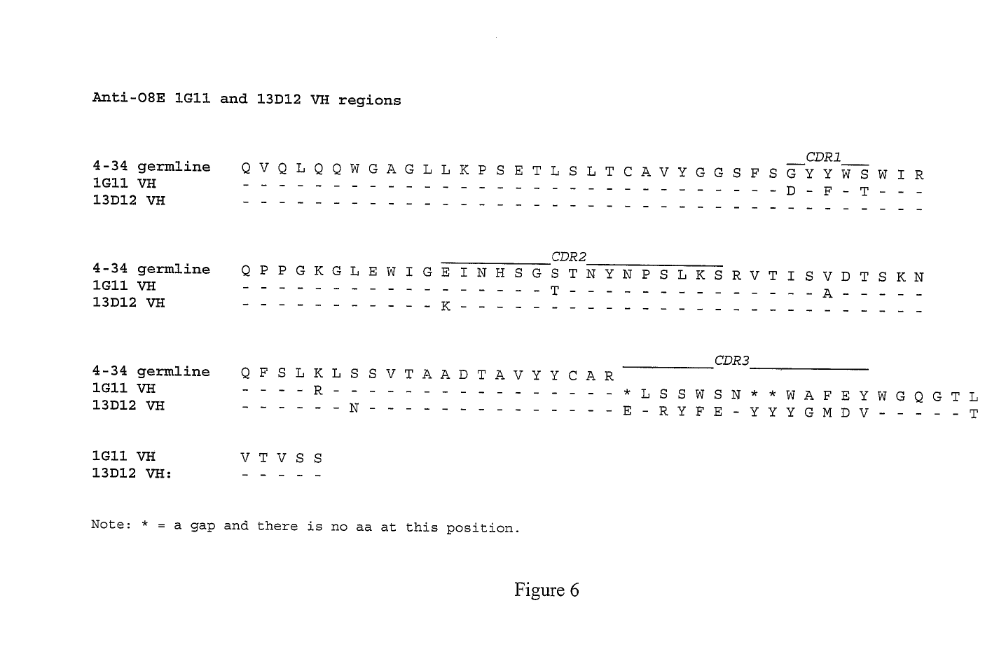

Comparison of the 1G11 heavy chain immunoglobulin sequence to the known human germline immunoglobulin heavy chain sequences demonstrated that the 1G11 heavy chain utilizes a VH segment from human germline VH 4-34. The alignment of the 1G11 VH sequence to the germline VH 4-34 sequence is shown in FIG. 6. Further analysis of the 1G11 VH sequence using the Kabat system of CDR region determination led to the delineation of the heavy chain CDR1, CDR2 and CD3 regions as shown in FIGS. 1A and 6 and in SEQ ID NOs: 11, 16 and 21, respectively.

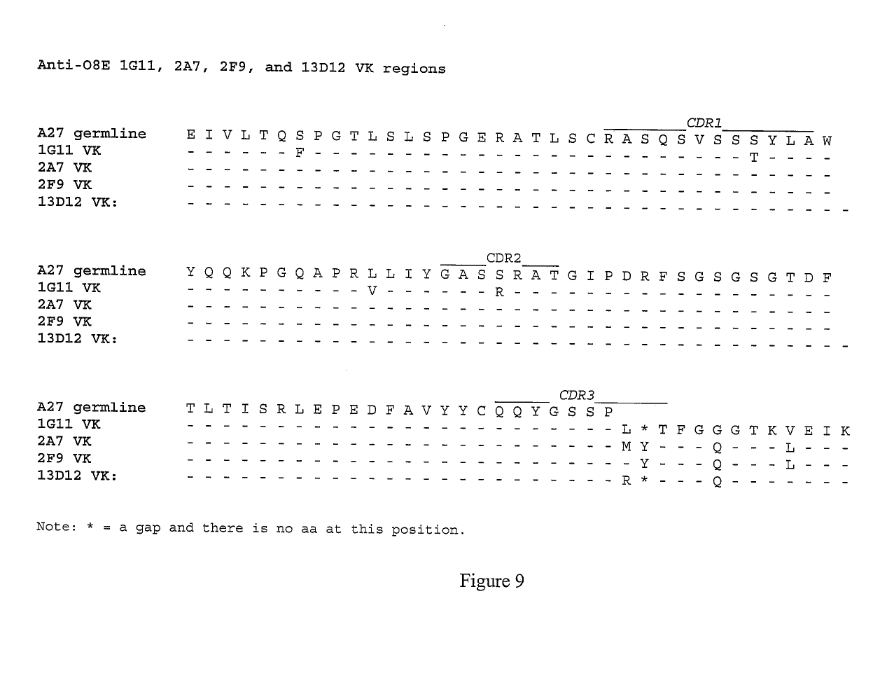

Comparison of the 1G11 light chain immunoglobulin sequence to the known human germline immunoglobulin light chain sequences demonstrated that the 1G11 light chain utilizes a VL segment from human germline VK A27. The alignment of the 1G11 VL sequence to the germline VK A27 sequence is shown in FIG. 9. Further analysis of the 1G11 VL sequence using the Kabat system of CDR region determination led to the delineation of the light chain CDR1, CDR2 and CD3 regions as shown in FIGS. 1B and 9 and in SEQ ID NOs: 26, 31 and 36, respectively.

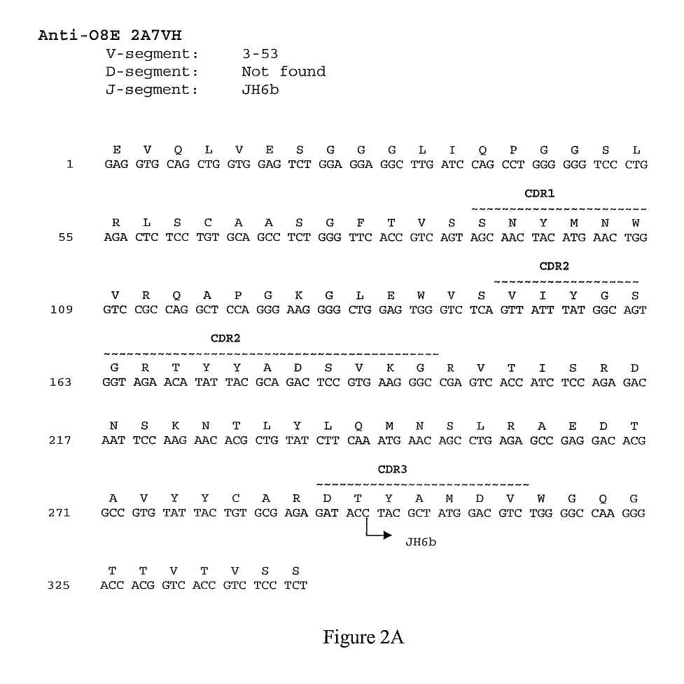

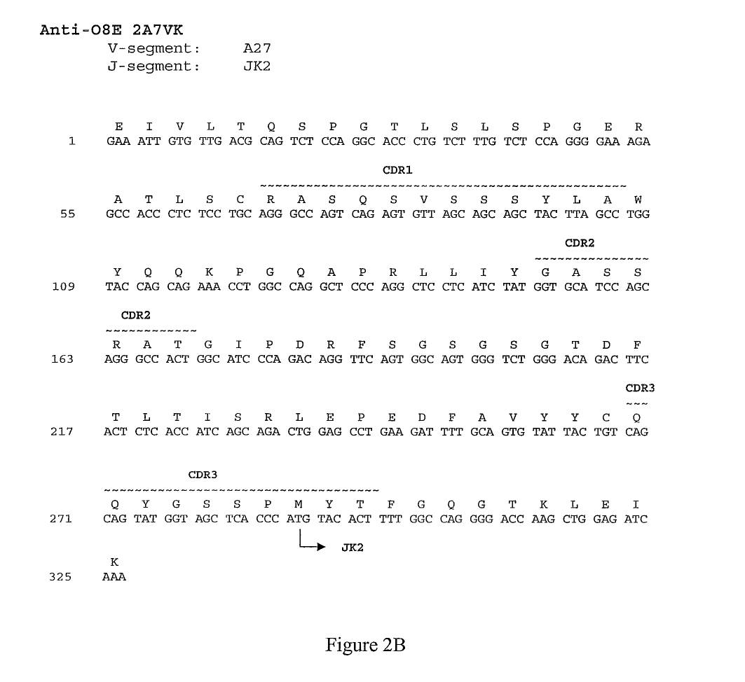

The nucleotide and amino acid sequences of the heavy chain variable region of 2A7 are shown in FIG. 2A and in SEQ ID NO: 42 and 2, respectively.

The nucleotide and amino acid sequences of the light chain variable region of 2A7 are shown in FIG. 2B and in SEQ ID NO: 47 and 7, respectively.

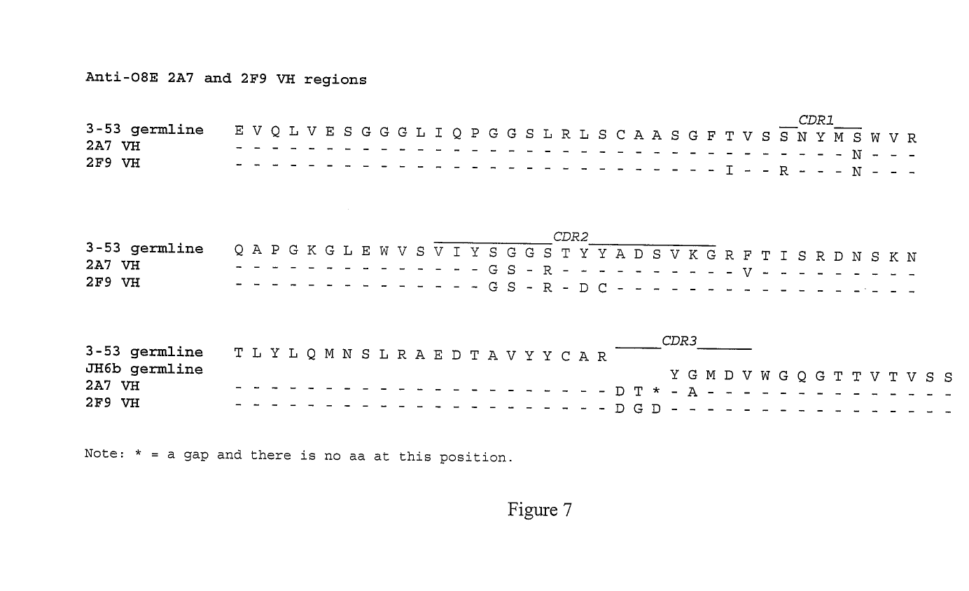

Comparison of the 2A7 heavy chain immunoglobulin sequence to the known human germline immunoglobulin heavy chain sequences demonstrated that the 2A7 heavy chain utilizes a VH segment from human germline VH 3-53 and a JH segment from human germline JH 6b. The alignment of the 2A7 VH sequence to the germline VH 3-53 sequence is shown in FIG. 7. Further analysis of the 2A7 VH sequence using the Kabat system of CDR region determination led to the delineation of the heavy chain CDR1, CDR2 and CD3 regions as shown in FIGS. 2A and 7 and in SEQ ID NOs: 12, 17 and 22, respectively.

Comparison of the 2A7 light chain immunoglobulin sequence to the known human germline immunoglobulin light chain sequences demonstrated that the 2A7 light chain utilizes a VL segment from human germline VK A27. The alignment of the 2A7 VL sequence to the germline VK A27 sequence is shown in FIG. 9. Further analysis of the 2A7 VL sequence using the Kabat system of CDR region determination led to the delineation of the light chain CDR1, CDR2 and CD3 regions as shown in FIGS. 2B and 9 and in SEQ ID NOs: 27, 32 and 37, respectively.

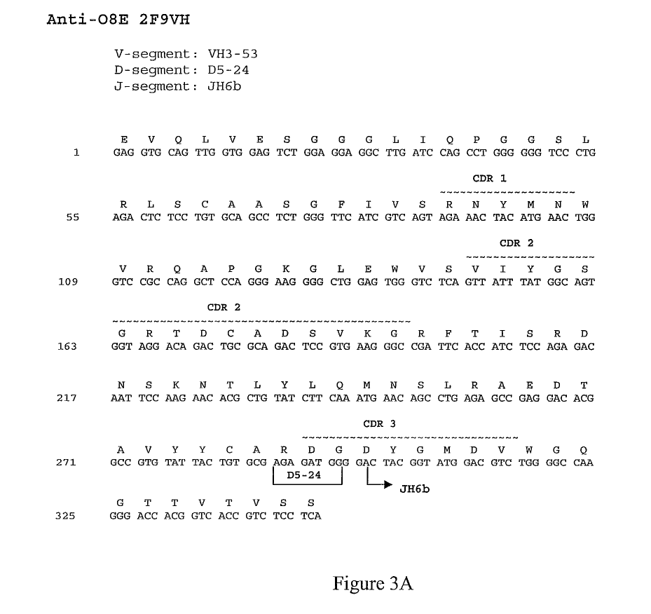

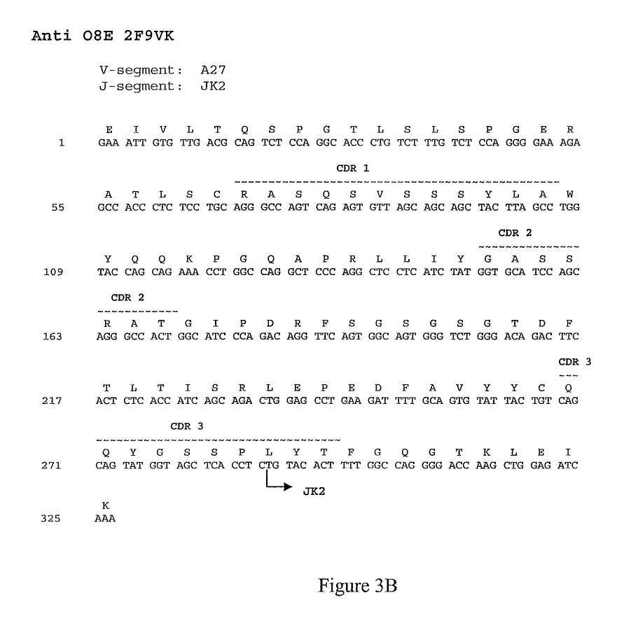

The nucleotide and amino acid sequences of the heavy chain variable region of 2F9 are shown in FIG. 3A and in SEQ ID NO: 43 and 3, respectively.

The nucleotide and amino acid sequences of the light chain variable region of 2F9 are shown in FIG. 3B and in SEQ ID NO: 48 and 8, respectively.

Comparison of the 2F9 heavy chain immunoglobulin sequence to the known human germline immunoglobulin heavy chain sequences demonstrated that the 2F9 heavy chain utilizes a VH segment from human germline VH 3-53 and a JH segment from human germline JH 6b. The alignment of the 2F9 VH sequence to the germline VH 3-53 sequence is shown in FIG. 7. Further analysis of the 2F9 VH sequence using the Kabat system of CDR region determination led to the delineation of the heavy chain CDR1, CDR2 and CD3 regions as shown in FIGS. 3A and 7 and in SEQ ID NOs: 13, 18 and 23, respectively.

Comparison of the 2F9 light chain immunoglobulin sequence to the known human germline immunoglobulin light chain sequences demonstrated that the 2F9 light chain utilizes a VL segment from human germline VK A27. The alignment of the 2F9 VL sequence to the germline VK A27 sequence is shown in FIG. 9. Further analysis of the 2F9 VL sequence using the Kabat system of CDR region determination led to the delineation of the light chain CDR1, CDR2 and CD3 regions as shown in FIGS. 3B and 9 and in SEQ ID NOs: 28, 33 and 38, respectively.

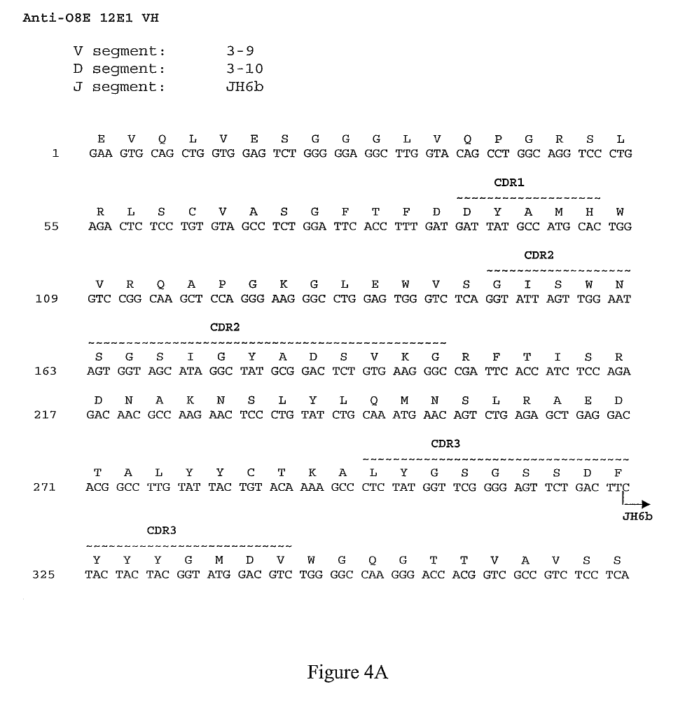

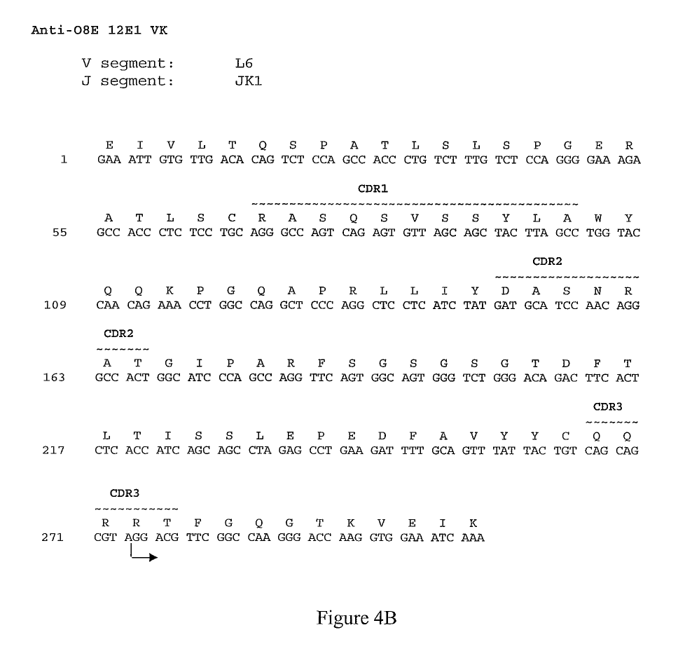

The nucleotide and amino acid sequences of the heavy chain variable region of 12E1 are shown in FIG. 4A and in SEQ ID NO: 44 and 4, respectively.

The nucleotide and amino acid sequences of the light chain variable region of 12E1 are shown in FIG. 4B and in SEQ ID NO: 49 and 9, respectively.

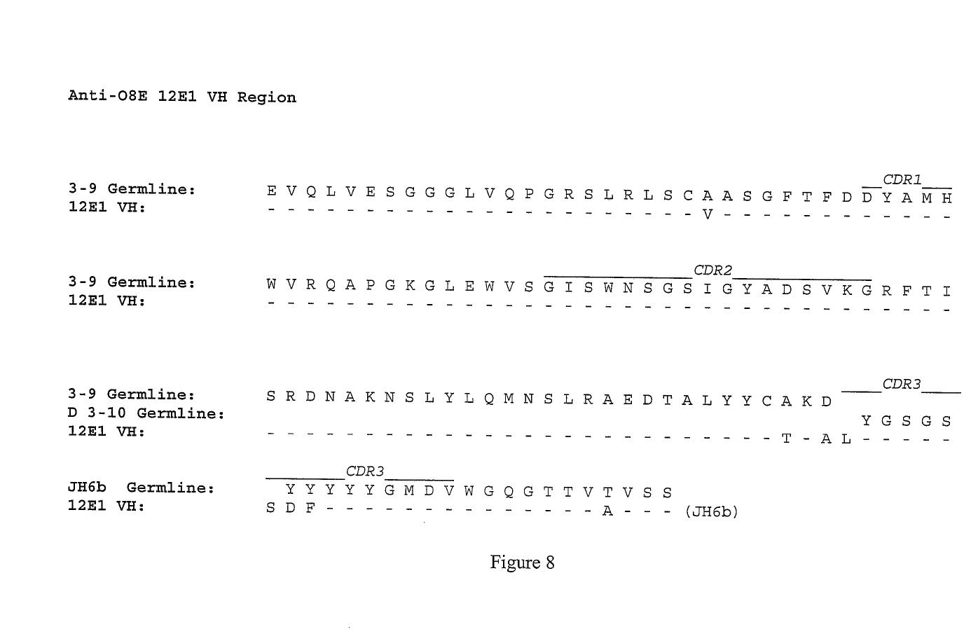

Comparison of the 12E1 heavy chain immunoglobulin sequence to the known human germline immunoglobulin heavy chain sequences demonstrated that the 12E1 heavy chain utilizes a VH segment from human germline VH 3-9, a D segment from human germline 3-10 and a JH segment from human germline JH 6b. The alignment of the 12E1 VH sequence to the germline VH 3-9 sequence is shown in FIG. 8. Further analysis of the 12E1 VH sequence using the Kabat system of CDR region determination led to the delineation of the heavy chain CDR1, CDR2 and CD3 regions as shown in FIGS. 3A and 8 and in SEQ ID NOs: 14, 19 and 24, respectively.

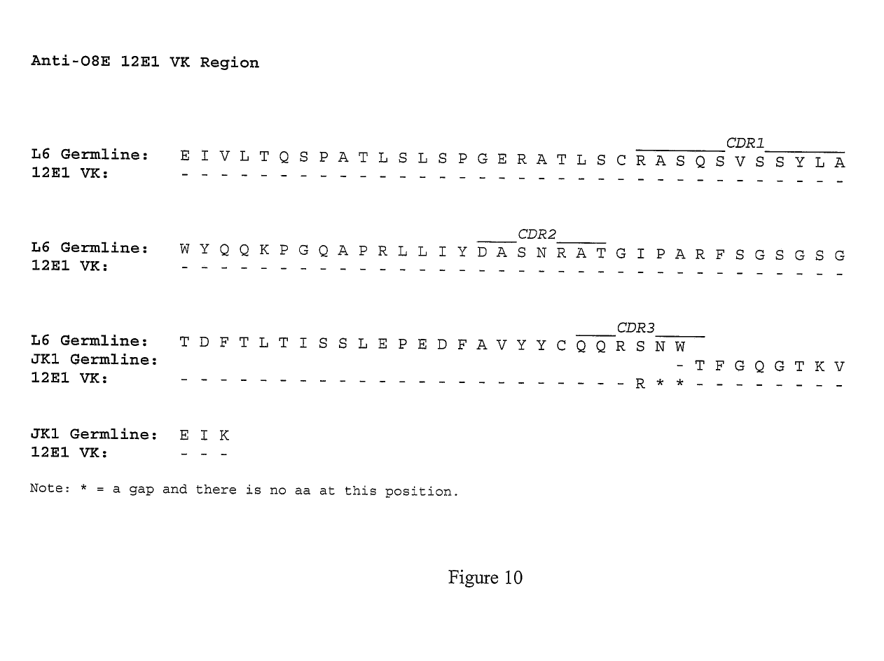

Comparison of the 12E1 light chain immunoglobulin sequence to the known human germline immunoglobulin light chain sequences demonstrated that the 12E1 light chain utilizes a VL segment from human germline VK L6 and a JK segment from human germline JK 1. The alignment of the 12E1 VL sequence to the germline VK L6 sequence is shown in FIG. 10. Further analysis of the 12E1 VL sequence using the Kabat system of CDR region determination led to the delineation of the light chain CDR1, CDR2 and CD3 regions as shown in FIGS. 3B and 10 and in SEQ ID NOs: 29, 34 and 39, respectively.

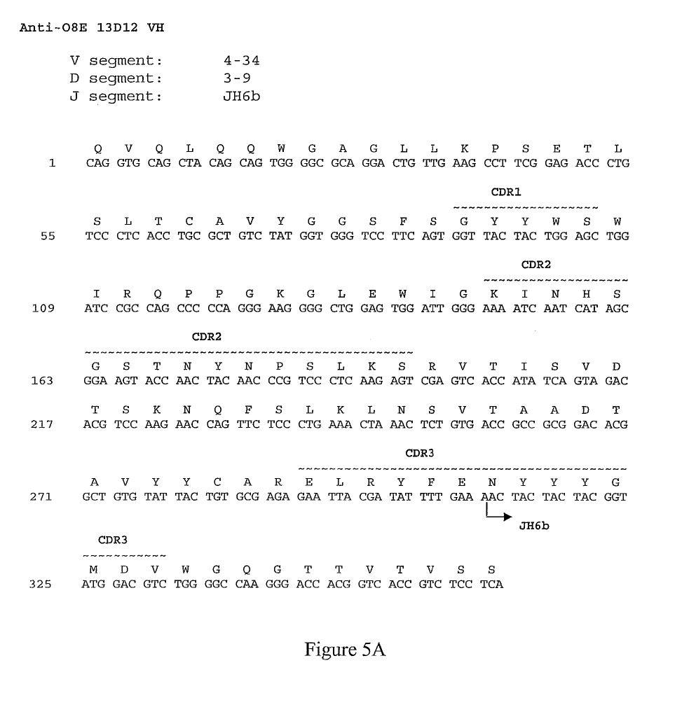

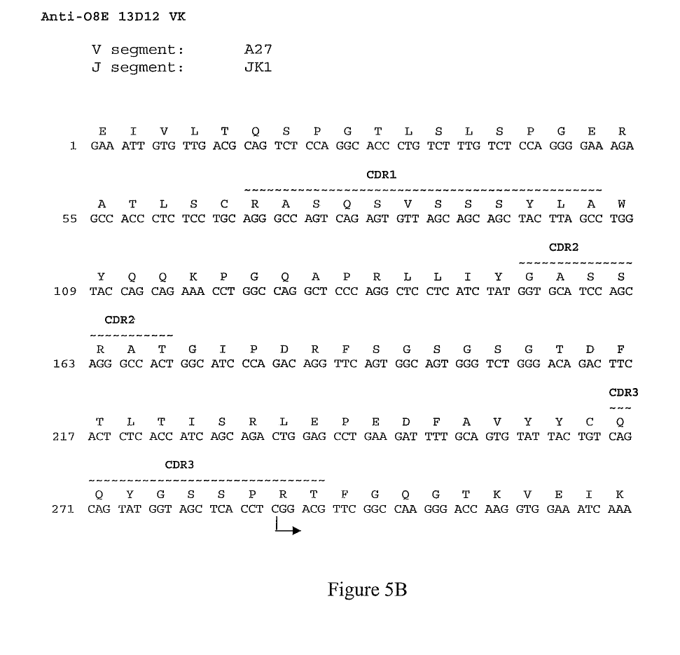

The nucleotide and amino acid sequences of the heavy chain variable region of 13D12 are shown in FIG. 5A and in SEQ ID NO: 45 and 5, respectively.

The nucleotide and amino acid sequences of the light chain variable region of 13D12 are shown in FIG. 5B and in SEQ ID NO: 50 and 10, respectively.

Comparison of the 13D12 heavy chain immunoglobulin sequence to the known human germline immunoglobulin heavy chain sequences demonstrated that the 13D12 heavy chain utilizes a VH segment from human germline VH 4-34. The alignment of the 13D12 VH sequence to the germline VH 4-34 sequence is shown in FIG. 6. Further analysis of the 13D12 VH sequence using the Kabat system of CDR region determination led to the delineation of the heavy chain CDR1, CDR2 and CD3 regions as shown in FIGS. 5A and 6 and in SEQ ID NOs: 15, 20 and 25, respectively.

Comparison of the 13D12 light chain immunoglobulin sequence to the known human germline immunoglobulin light chain sequences demonstrated that the 13D12 light chain utilizes a VL segment from human germline VK A27. The alignment of the 13D12 VL sequence to the germline VK A27 sequence is shown in FIG. 9. Further analysis of the 13D12 VL sequence using the Kabat system of CDR region determination led to the delineation of the light chain CDR1, CDR2 and CD3 regions as shown in FIGS. 5B and 9 and in SEQ ID NOs: 30, 35 and 40, respectively.

### Example 3

### Characterization of Binding Specificity of Anti-O8E Human Monoclonal Antibodies

This Example discloses a comparison of anti-O8E antibodies on binding to immunopurified O8E performed by standard ELISA to examine the specificity of binding for O8E.

Recombinant His-tagged and myc-tagged O8E was coated on a plate overnight., then tested for binding against the anti-O8E human monoclonal antibodies 2A7, 12E1 and 13D12.

Standard ELISA procedures were performed. The anti-O8E human monoclonal antibodies were added at a concentration of 1 μg/ml and titrated down at 1:2 serial dilutions. Goat-anti-human IgG (Fc or kappa chain-specific) polyclonal antibody conjugated with horseradish peroxidase (HRP) was used as secondary antibody.

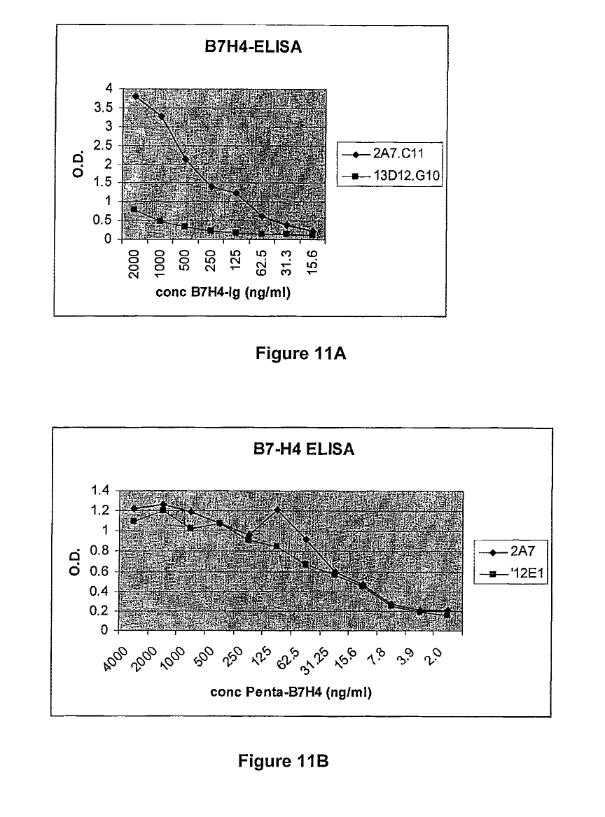

Recombinant B7H4-Ig was purified from supernatants of 293T cells transfected with a B7H4-Ig construct by chromatography using protein A. An ELISA plate was coated with the human antibodies, followed by addition of purified protein and then detection with the rabbit anti-B7H4 antisera. See, FIG. 11A. Recombinant Penta-B7H4 protein with a C-9 tag was purified from supernatants of 293T cells transfected with a Penta-B7H4-C9 construct by chromatography using a 2A7 affinity column. An ELISA plate was coated with anti-mouse Fc, followed by monoclonal anti-C9 (0.6 ug/ml), then titrated Penta-B7H4 as indicated, then the human antibodies at 1 ug/ml. The plate was coated with anti-mouse Fc, followed by M-anti-C9 (0.6 ug/ml), and then was titrated using Penta-O8E as indicated, then with humabs at 1 ug/ml. See, e.g., FIG. 11B.

The anti-O8E human monoclonal antibodies 2A7, 12E1 and 13D12 bound with high specificity to O8E.

### Example 4

### Characterization of Anti-O8E Antibody Binding to O8E Expressed on the Surface of Breast Cancer Carcinoma Cell Lines

This Example discloses the testing of anti-O8E antibodies for binding to CHO-O8E (a/k/a B7H4, B7S1 and B7x) transfectants and breast cell carcinoma cells expressing O8E on their cell surface by flow cytometry.

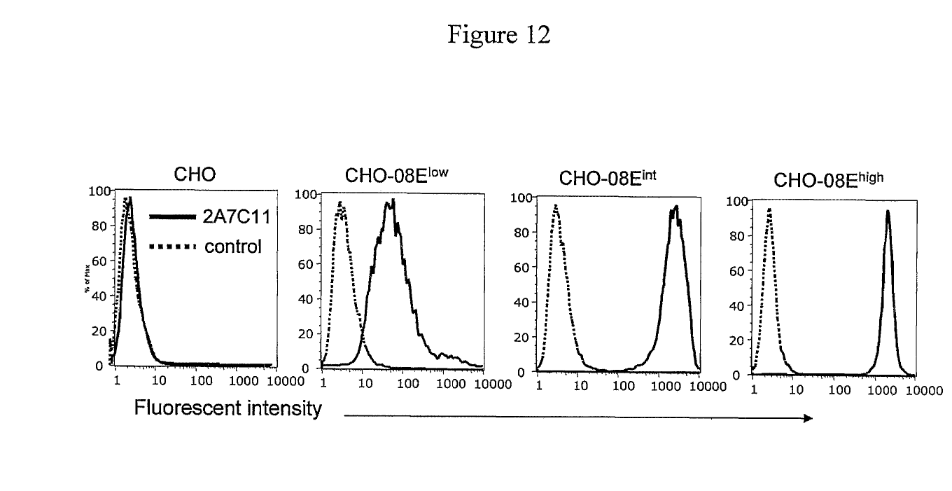

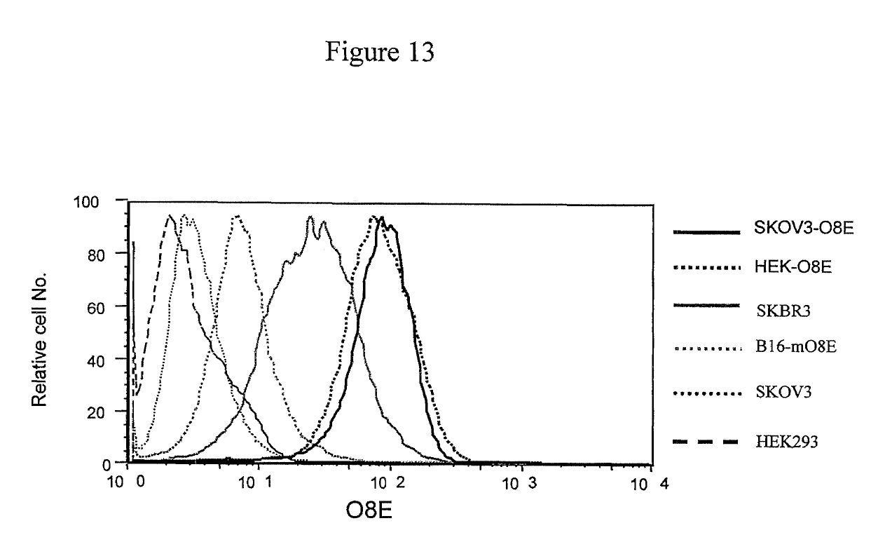

A CHO cell line transfected with O8E as well as the breast cell carcinoma cell line SKBR3 (ATCC Accession No. HTB-30) were tested for antibody binding. Binding of the HuMAb 2A7 anti-O8E human monoclonal antibody was assessed by incubating 1×10⁵ cells with 2A7 at a concentration of 1 μg/ml. The cells were washed and binding was detected with a FITC-labeled anti-human IgG Ab. Flow cytometric analyses were performed using a FACScan flow cytometry (Becton Dickinson, San Jose, Calif.). The results are shown in FIGS. 12 and 13.

These data demonstrate that the anti-O8E HuMAbs bind to O8E expressing CHO cells and to an exemplary breast cell carcinoma cell line.

### Example 5

### Scatchard Analysis of Binding Affinity of Anti-O8E Monoclonal Antibodies

This Example discloses the testing of human monoclonal antibodies 1G11, 2F9, 2A7, 12E1 and 13D12 monoclonal antibodies for binding affinity to a O8E transfected HEK cell line using a Scatchard analysis.

HEK cells were transfected with full length O8E using standard techniques and grown in RPMI media containing 10% fetal bovine serum (FBS). (FIG. 12 presents FACs analysis of these HEK-O8E cells with the 2A7 human anti-O8E monoclonal antibody.) The cells were trypsinized and washed once in Tris based binding buffer (24 mM Tris pH 7.2, 137 mM NaCl, 2.7 mM KCl, 2 mM Glucose, 1 mM CaCl₂, 1 mM MgCl₂, 0.1% BSA) and the cells were adjusted to 2×10⁶ cells/ml in binding buffer. Millipore plates (MAFB NOB) were coated with 1% nonfat dry milk in water and stored a 4° C. overnight. The plates were washed three times with 0.2 ml of binding buffer. Fifty microliters of buffer alone was added to the maximum binding wells (total binding). Twenty-five microliters of buffer alone was added to the control wells (non-specific binding). Varying concentration of ¹²⁵I-anti-O8E antibody was added to all wells in a volume of 25 μl. (In some cases FITC labeled antibodies were used for the titration since unlabeled material was not available, binding may be compromised in these instances.) Varying concentrations of unlabeled antibody at 100 fold excess was added in a volume of 25 μl to control wells and 25 μl of O8E transfected CHO cells (2×10⁶ cells/ml) in binding buffer were added to all wells. The plates were incubated for 2 hours at 200 RPM on a shaker at 4° C. At the completion of the incubation the Millipore plates were washed three times with 0.2 ml of cold wash buffer (24 mM Tris pH 7.2, 500 mM NaCl, 2.7 mM KCl, 2 mM Glucose, 1 mM CaCl₂, 1 mM MgCl₂, 0.1% BSA.). The filters were removed and counted in a gamma counter. Evaluation of equilibrium binding was performed using single site binding parameters with the Prism software (San Diego, Calif.).

Data were analyzed by non-linear regression using a sigmoidal dose response (PRIZM™) and resulted in calculation of an EC50, which was used to rank the antibodies as illustrated in Table 2. The EC50 values calculated in these experiments are qualitative measures of antibody affinity and do not represent absolute affinities for O8E.

TABLE 2

| Antibody | EC50 | 95% CI |
|---|---|---|
| 2F9.E6-FITC | 407 pM | 250 to 663 pM |
| 13D12.G10 | 746 pM | 569 to 979 pM |
| 2A7.C11 | 750 pM | 519 pM to 1 nM |
| 1G11.H11-FITC | 1.69 nM | 1.4 to 2.0 nM |
| 12E1.G9* | 19.8 pM | 14 to 27.6 nM |

*BOTTOM and TOP values adjusted as constants to compensate for incomplete curve.

### Example 6

### Internalization of Anti-O8E Monoclonal Antibody

This Example demonstrates the testing of anti-O8E HuMAbs for the ability to internalize into O8E-expressing CHO and breast carcinoma cells using a Hum-Zap internalization assay. The Hum-Zap assay tests for internalization of a primary human antibody through binding of a secondary antibody with affinity for human IgG conjugated to the toxin saporin.

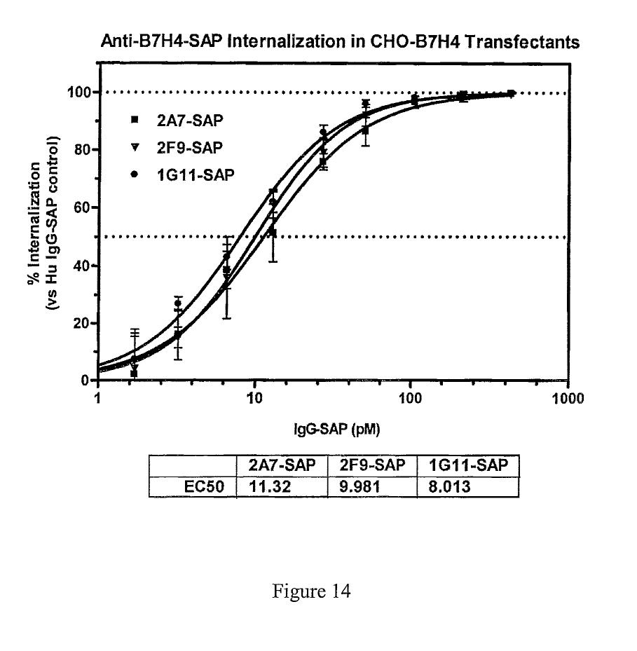

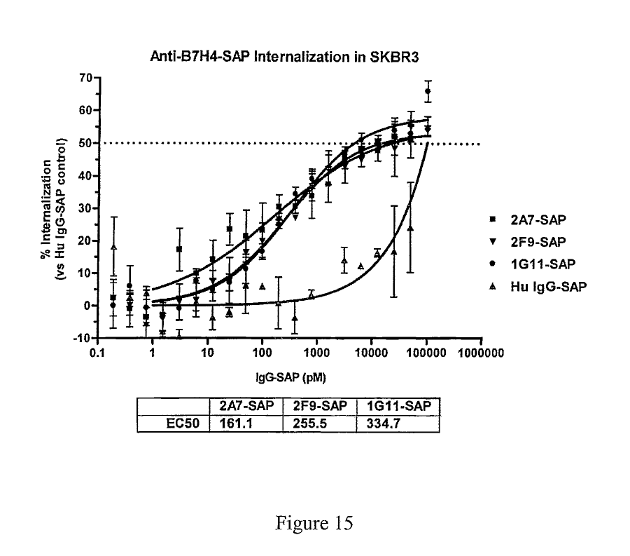

The O8E-expressing breast carcinoma cancer cell line SKBR3 was seeded at 1.25×10⁴ cells/well in 100 μl wells overnight. The anti-O8E HuMAb antibodies 1G11, 2F9, 2A7, 12E1 or 13D12 were added to the wells at a concentration of 10 pM. An isotype control antibody that is non-specific for O8E was used as a negative control. The Hum-Zap (Advanced Targeting Systems, San Diego, Calif., IT-22-25) was added at a concentration of 11 nM and plates were allowed to incubate for 72 hours. The plates were then pulsed with 1.0 μCi of ³H-thymidine for 24 hours, harvested and read in a Top Count Scintillation Counter (Packard Instruments, Meriden, Conn.). The results are presented below in Table 3 and in FIGS. 14-15. The anti-O8E antibodies 1G11, 2F9, 2A7, 12E1 and 13D12 showed an antibody concentration dependent decrease in ³H-thymidine incorporation in O8E-expressing SKBR3 breast carcinoma cancer cells.

These data demonstrate that the anti-O8E antibodies 1G11, 2F9, 2A7, 12E1 and 13D12 internalize into a breast carcinoma cancer cell line.

TABLE 3

| | Assay No. 1 | | Assay No. 2 | | Assay No. 3 | |
|---|---|---|---|---|---|---|
| | % internal- | | % internal- | | % internal- | |
| | ization | | ization | | ization | |
| Anti-O8E | mean | sd | mean | sd | mean | sd |
| 2A7/C11 | 29 | 12 | 17.5 | 3.5 | 40.7 | 2.7 |
| 2F9.E6 | 37 | 17 | NT | NT | NT | NT |
| 1G11.H1 | 18 | 8 | NT | NT | NT | NT |
| 13D12.G10 | NT | NT | 12.1 | 2.5 | 12.2 | 2.8 |
| 12E1.G9 | NT | NT | 10.4 | 18.5 | 4.3 | 2.7 |

The ranking for internalization efficiency was averaged over three experiments in SKBR3 and two experiments in CHO-O8E. The internalization rankings, along with EC50s for binding to CHO-O8E, are presented in Tables 4 and 5. Results show that internalization efficiency does not directly correlate with binding affinity, which suggests that internalization is epitope dependant.

TABLE 4
Internalization Efficiency Sorted by Internalization in
the SBKR3 Breast Carcinoma Cell Line

| | | | EC50 | |
|---|---|---|---|---|
| | Internalization | | CHO-O8E | |
| Anti-O8E | SKBR3 | CHO-O8E | binding | |
| 2F9.E6 | 1 | 3 | 407 | pM |
| 2A7.C11 | 2 | 1 | 750 | pM |
| 1G11.H1 | 3 | 4 | 1.69 | nM |
| 13D12.G10 | 4 | 2 | 746 | pM |
| 12E1.G9 | 5 | 5 | 19.8 | pM |

TABLE 5
Internalization Efficiency Sorted by Internalization
in the CHO-O8E Cell Line

| | | | EC50 | |
|---|---|---|---|---|
| | Internalization | | CHO-O8E | |
| Anti-O8E | SKBR3 | CHO-O8E | binding | |
| 2A7.C11 | 2 | 1 | 750 | pM |
| 13D12.G10 | 4 | 2 | 746 | pM |
| 2F9.E6 | 1 | 3 | 407 | pM |
| 1G11.H1 | 3 | 4 | 1.69 | nM |
| 12E1.G9 | 5 | 5 | 19.8 | pM |

The internalization activity of the saporin conjugates in CHO-O8E was measured with a dose response through a ˜500 pM to 1 pM range using human monoclonal antibodies 2A7, 2F9 and 1G11. As illustrated in FIG. 14, internalization was very efficient with EC50s in the low pM range. A CHO parental cell line and Hu IgG-SAP were used as negative controls and showed no significant background toxicity or non-specific internalization. Direct anti-O8E conjugates to SAP were used with SKBR3 cells. The percentage of internalization (vs control) as a function of Ig-SAP dose is presented in FIG. 15.

### Example 7

### Assessment of Cell Killing of a Toxin-Conjugated Anti-O8E Antibody on Breast Cell Carcinoma Cell Lines

This Example discloses the testing of anti-O8E monoclonal antibodies conjugated to a toxin for the ability to kill an O8E⁺ breast cell carcinoma cell line in a cell proliferation assay.

The anti-O8E HuMAb antibodies 1G11, 2F9, 2A7, 12E1 or 13D12 may be conjugated to a toxin via a linker, such as a peptidyl, hydrazone or disulfide linker. An O8E-expressing breast carcinoma cancer cell line, such as SKBR3, is seeded at between about 1 and 3×10⁴ cells/wells in 100 μl wells for 3 hours. An anti-O8E antibody-toxin conjugate is added to the wells at a starting concentration of 30 nM and titrated down at 1:3 serial dilutions. An isotype control antibody that is non-specific for O8E is used as a negative control. Plates are allowed to incubate for 69 hours. The plates are then pulsed with 1.0 of ³H-thymidine for 24 hours, harvested and read in a Top Count Scintillation Counter (Packard Instruments, Meriden, Conn.). Anti-O8E antibodies are expected to show an antibody-toxin concentration dependent decrease in ³H-thymidine incorporation in O8E-expressing breast carcinoma cancer cells. This data demonstrates that the anti-O8E antibodies 1G11, 2F9, 2A7, 12E1 and 13D12 are potentially cytotoxic to breast carcinoma cancer cells when conjugated to a toxin.

### Example 8

### Assessment of ADCC Activity of Anti-O8E Antibody

This Example discloses the testing of anti-O8E monoclonal antibodies for the ability to kill O8E⁺ cell lines in the presence of effector cells via antibody dependent cellular cytotoxicity (ADCC) in a fluorescence cytotoxicity assay.

Human effector cells were prepared from whole blood as follows. Human peripheral blood mononuclear cells were purified from heparinized whole blood by standard Ficoll-paque separation. The cells were resuspended in RPMI1640 media containing 10% FBS and 200 U/ml of human IL-2 and incubated overnight at 37° C. The following day, the cells were collected and washed four times in culture media and resuspended at 2×10⁷ cells/ml. Target O8E⁺ cells were incubated with BATDA reagent (Perkin Elmer, Wellesley, Mass.) at 2.5 BATDA per 1×10⁶ target cells/mL for 20 minutes at 37° C. The target cells were washed four times, spun down and brought to a final volume of 1×10⁵ cells/ml.

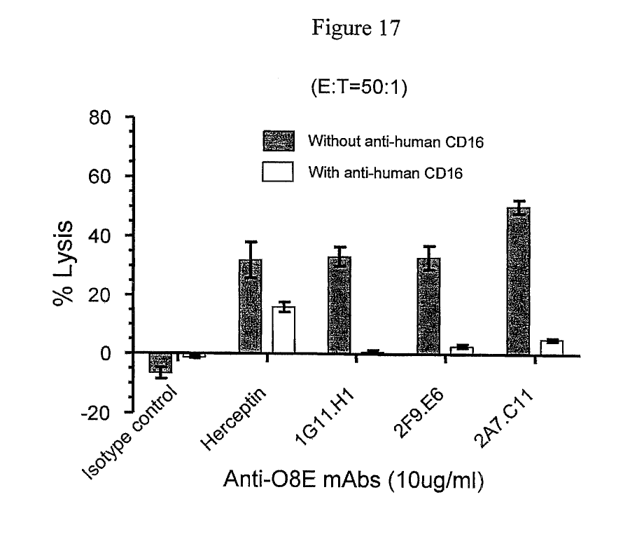

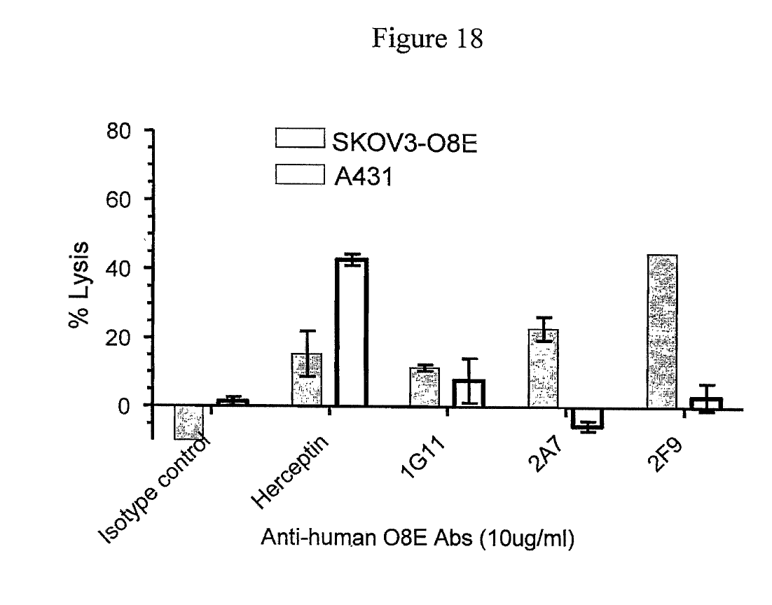

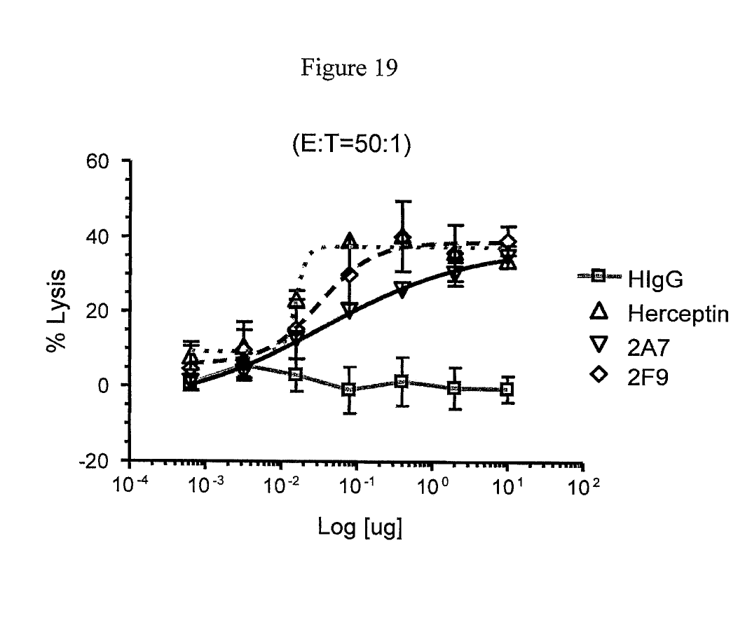

The O8E⁺ cell line SKBR3 as well as an O8E transfected SKOV3 cell-line were tested for antibody specific ADCC to the human anti-O8E monoclonal antibodies using the Delfia fluorescence emission analysis as follows. Each target cell line (100 μl of labeled target cells) was incubated with 50 μl of effector cells and 50 μl of antibody. A target to effector ratio of 1:50 was used throughout the experiments. In all studies, a human IgG1 isotype control was used as a negative control. Following a 2000 rpm pulse spin and one hour incubation at 37° C., the supernatants were collected, quick spun again and 20 of supernatant was transferred to a flat bottom plate, to which 180 μl of Eu solution (Perkin Elmer, Wellesley, Mass.) was added and read in a RubyStar reader (BMG Labtech). The % lysis was calculated as follows: (sample release−spontaneous release*100)/(maximum release−spontaneous release), where the spontaneous release is the fluorescence from wells which only contain target cells and maximum release is the fluorescence from wells containing target cells and have been treated with 2% Triton-X. Cell cytotoxicity % lysis for the SKBR3 cells with anti-O8E antibodies 1G11, 2F9 and 2A7 are presented in FIG. 17; cell cytotoxicity % lysis for the SKOV3-O8E transfected cell line with anti-O8E antibodies 1G11, 2F9 and 2A7 are presented in FIG. 18; and concentration-dependent cell cytotoxicity % lysis for the SKBR3 cells with anti-O8E antibodies 2F9 and 2A7 are presented in FIG. 19. Both of the O8E⁺-expressing cell lines SKBR3 and SKOV3-O8E showed antibody mediated cytotoxicity with the HuMAb anti-O8E antibodies 1G11, 2F9 and 2A7. These data demonstrate that HuMAb anti-O8E antibodies show specific cytotoxicity to O8E⁺ expressing cells.

### Example 9

### Treatment of In Vivo Tumor Xenograft Model Using Naked and Cytotoxin-Conjugated Anti-O8E Antibodies

This Example discloses the in vivo treatment of mice implanted with a breast cell carcinoma tumor with toxin-conjugated anti-O8E antibodies to examine the in vivo effect of the antibodies on tumor growth.

SKBR3 or other suitable breast cell carcinoma cells are expanded in vitro using standard laboratory procedures. Male Ncr athymic nude mice (Taconic, Hudson, N.Y.) between 6-8 weeks of age are implanted subcutaneously in the right flank with 7.5×10⁶ ACHN or A-498 cells in 0.2 ml of PBS/Matrigel (1:1) per mouse. Mice are weighed and measured for tumors three dimensionally using an electronic caliper twice weekly after implantation. Tumor volumes are calculated as height×width×length. Mice with ACHN tumors averaging 270 mm³ or A498 tumors averaging 110 mm³ are randomized into treatment groups. The mice are dosed intraperitoneally with PBS vehicle, toxin-conjugated isotype control antibody or toxin-conjugated anti-O8E HuMAb on Day 0. Examples of toxin compounds that may be conjugated to the antibodies of the current disclosure are described in pending U.S. Patent Application designated MEDX-0034US4. The mice receiving anti-O8E HuMAb are tested with three different toxin compounds. Mice are monitored for tumor growth for 60 days post dosing. Mice are euthanized when the tumors reached tumor end point (2000 mm³). Suitable anti-O8E antibodies conjugated to a toxin extend the mean time to reaching the tumor end point volume (2000 mm³) and slow tumor growth progression. Thus, treatment with such an anti-O8E antibody-toxin conjugate has a direct in vivo inhibitory effect on tumor growth.

### Example 10

### Immunohistochemishy with Anti-O8E HuMAb 2A7

This Example discloses that the anti-O8E HuMAb 2A7 to recognize O8E by immunohistochemistry using normal mouse tissue arrays (IMGENEX Histo-Array; Imgenex Corp., San Diego, Calif.).

For immunohistochemistry, 2,000 μm tissue cores were used. After drying for 30 minutes, sections were fixed with acetone (at room temperature for 10 minutes) and air-dried for 5 minutes. Slides were rinsed in PBS and then pre-incubated with 10% normal goat serum in PBS for 20 min and subsequently incubated with 10 μg/ml fitcylated 2A7 in PBS with 10% normal goat serum for 30 min at room temperature. Next, slides were washed three times with PBS and incubated for 30 min with mouse anti-FITC (10 μg/ml DAKO) at room temperature. Slides were washed again with PBS and incubated with Goat anti-mouse HRP conjugate (DAKO) for 30 minutes at room temperature. Slides were washed again 3× with PBS. Diaminobenzidine (Sigma) was used as substrate, resulting in brown staining. After washing with distilled water, slides were counter-stained with hematoxyllin for 1 min.

Subsequently, slides were washed for 10 secs in running distilled water and mounted in glycergel (DAKO). The results of these studies are presented in Table 6.

TABLE 6
Immunoreactivity of O8E in Normal Mouse Tissue Array

| | 2A7.C11-FITC | | Hu-IgG1-FITC |
|---|---|---|---|
| Tissue Types | 2 μg/ml | 5 μg/ml | 5 μg/ml |
| Skin, ear lobe | | | |
| Epidermis | − | ± | − |
| Sebaceous gland | − | ± | − |
| Other elements | − | − | − |
| Colon | | | |
| Surface epithelium | ±,1+ | 1+ | ± |
| Other elements | − | − | − |
| Small Intestine | | | |
| Crypt epithelium | ±, 1+ | 1+, 2+ | ± |
| Other elements | − | − | − |
| Stomach | | | |
| Surface & glandular | 1+, 2+, ocas | 1+, 2+, freq | 1+, 2+, ocas |
| epithelial cells | | | |
| Nerve plexus | − | ±, 1+ | − |
| Other elements | − | − | − |
| Pancreas | | | |
| Acinar epithelium | 1+ | 2+ | ±, 1+ |
| Islets | − | ± | − |
| Other elements | − | − | − |
| Salivary gland | | | |
| Acinar epithelium | ± | 1+ | − |
| Other elements | − | − | − |
| Liver | | | |
| Hepatocytes | − | ±, − | − |
| Other elements | − | − | − |
| Cerebrum | | | |
| Neurons | ± | 2+, 1+, freq | ±, − |
| Neuropil/fibers | −, ± | 2+, 1+, ocas | − |
| Pons | | | |
| Neurons | ± | ± | ± |
| Neuropil/fibers | ± | 2+, 1+, freq | − |
| Cerebelleum | | | |
| Purkinje cells | ±, 1+ | 1+ | ±, − |
| White matter | − | 1+, 2+ | − |
| Other elements | − | − | − |
| Spleen | | | |
| Large lymphoid cells | − | 1+, 2+, rare | − |
| in red pulp | | | |
| Other elements | − | −, ± | − |
| Thymus | − | − | − |
| Skeletal muscle | − | − | − |
| Tongue | − | − | − |
| Heart | − | −, ± | − |
| Lung | − | − | − |
| Kidney cortex | − | −, ± | − |
| Kidney medulla | − | − | − |
| Urinary bladder | | | |
| Transitional epithelium | − | ±, 1+ | − |
| Other elements | − | − | − |
| Seminal vesicle | | | |
| Epithelium | ±, − | ± | − |
| Fluid in the lumen | 1+ | 3+ | ± |
| Other elements | − | − | − |
| Testis | | | |
| Primary Spermotocytes | − | ±, 1+ | − |
| Other elements | − | − | − |
| Epididymis | − | − | − |
| Uterus | | | |
| Endometrium/gland | −, ± | ± | − |
| epithelium | | | |
| Other elements | − | − | − |
| Ovary | − | ± | − |

Intensity of immunoreactivity: +− (equivocal); + (weak); 2+ (moderate); 3+ (strong); 4+ (intense); − (negative). Freq: frequent; Ocas: occasional

These data and corresponding data collected for anti-O8E antibodies 1G11 and 2F9, demonstrate that strong to intense O8E immunoreactivity (3+, 4+) was present in enteroendocrine-like cells in colon and small intestine, as well as in the lumen fluid of seminary vesicle; weak to moderate O8E immunoreactivity (1+, 2+) was revealed in neurons of cerebrum, in neuropils and fibers of cerebrum and pons, in the white matter of cerebellum, in the crypt epithelial cells of small intestine and in a small number of large lymphoid cells in the spleen; weak O8E immunoreactivity (1+) was demonstrated in colon surface epithelium, Purkinje cells in cerebellum and acinar epithelium of salivary gland and pancreas; equivocal to weak O8E immunoreactivity was shown in transitional epithelium of urinary bladder, primary spermotocytes of testis and nerve plexus in stomach; and all other organs exhibit negative to equivocal staining, which include skin, liver, heart, lung, thymus, kidney, uterus, ovary, epididymis, tongue and skeletal muscles.

### Example 11

### Production of Defucosylated HuMAbs

This Example demonstrates the production of anti-O8E HuMAbs lacking in fucosyl residues.

Antibodies with reduced amounts of fucosyl residues have been demonstrated to increase the ADCC ability of the antibody. The CHO cell line Ms704-PF, which lacks the fucosyltransferase gene FUT 8 (Biowa, Inc., Princeton, N.J.), is electroporated with a vector that expresses the heavy and light chains of an anti-O8E HuMAb. Drug-resistant clones are selected by growth in Ex-Cell 325-PF CHO media (JRH Biosciences, Lenexa, Kans.) with 6 mM L-glutamine and 500 μg/ml G418 (Invitrogen, Carlsbad, Calif.). Clones are screened for IgG expression by standard ELISA assay. Two separate clones are produced, B8A6 and B8C11, which has production rates ranging from 1.0 to 3.8 picograms per cell per day.

### Example 12

### Assessment of ADCC Activity of Defucosylated Anti-O8E Antibody

This Example discloses the testing of defucosylated and non-defucosylated anti-O8E monoclonal antibodies for the ability to kill O8E⁺ cells in the presence of effector cells via antibody dependent cellular cytotoxicity (ADCC) in a fluorescence cytotoxicity assay.

Human anti-O8E monoclonal antibodies are defucosylated as described above. Human effector cells are prepared from whole blood as follows. Human peripheral blood mononuclear cells are purified from heparinized whole blood by standard Ficoll-paque separation. The cells are resuspended in RPMI1640 media containing 10% FBS (culture media) and 200 U/ml of human IL-2 and incubated overnight at 37° C. The following day, the cells are collected and washed once in culture media and resuspended at 2×10⁷ cells/ml. Target O8E+ cells are incubated with BATDA reagent (Perkin Elmer, Wellesley, Mass.) at 2.5 μl BATDA per 1×10⁶ target cells/mL in culture media supplemented with 2.5 mM probenecid (assay media) for 20 minutes at 37° C. The target cells are washed four times in PBS with 20 mM HEPES and 2.5 mM probenecid, spun down and brought to a final volume of 1×10⁵ cells/ml in assay media.

The O8E+ cell line ARH-77 (human B lymphoblast leukemia; ATCC Accession No. CRL-1621) is tested for antibody specific ADCC to the defucosylated and non-defucosylated human anti-O8E monoclonal antibody using the Delfia fluorescence emission analysis as follows. The target cell line ARH77 (100 μl of labeled target cells) is incubated with 50 μl of effector cells and 50 μl of either 1G11 or defucosylated 1G11 antibody. A target to effector ratio of 1:100 is used throughout. A human IgG1 isotype control is used as a negative control. Following a 2100 rpm pulse spin and one hour incubation at 37° C., the supernatants are collected, quick spun again and 20 μl of supernatant is transferred to a flat bottom plate, to which 180 μl of Eu solution (Perkin Elmer, Wellesley, Mass.) is added and read in a Fusion Alpha TRF plate reader (Perkin Ehner). The % lysis is calculated as follows: (sample release−spontaneous release*100)/(maximum release−spontaneous release), where the spontaneous release is the fluorescence from wells which only contain target cells and maximum release is the fluorescence from wells containing target cells and have been treated with 3% Lysol. The O8E+expressing cell line ARH-77 will show an antibody mediated cytotoxicity with the HuMAb anti-O8E antibody 1G11 and an increased percentage of specific lysis associated with the defucosylated form of the anti-O8E antibody 1G11. Thus, defucosylated HuMAb anti-O8E antibodies increase specific cytotoxicity to O8E+expressing cells.

### Example 13

### Internalization of HuMab Anti-O8E Antibodies by Immuno-Fluorescence Staining Analysis

The target cell lines, O8E⁺ SKBR3 (human breast cancer, ATCC #HTB-30) and ZR-75 (human breast cancer, ATCC #CRL-1500) were used to test for internalization of HuMab anti-O8E antibodies 2A7C11, 1G11H1 and 2F9E6 upon binding to the cells using immuno-fluorescence staining.

SKBR3 and ZR-75 cells (10⁴ per 100 μl per well in 96-well plate), harvested from tissue culture flask by treatment with 0.25% Trypsin/EDTA, were incubated with each of HuMab anti-O8E antibodies at 5 μg/ml in FACS buffer (PBS+5% FBS, media) for 30 minutes on ice. A human IgG1 isotype control was used as a negative control. Following 2× washes with the media, the cells were re-suspended in the media (100 μl per well) and then incubated with goat anti-human secondary antibody conjugated with PE (Jackson ImmunoResearch Lab) on ice for 30 minutes. Following washed with the media, the cells were either immediately imaged under a fluorescent microscope (Nikon) at 0 min or incubated at 37° C. for various times. The images of cell morphology and immuno-fluorescence intensity of the stained cells were taken at different time points as indicated in the figures below. The fluorescence was only observed in the cells stained with HuMab anti-O8E antibodies. No fluorescence was detected with the IgG1 control antibody. Similar results were also obtained with FITC-direct conjugated HuMab anti-O8E antibodies in the assays.

The imaging data showed the appearance of the fluorescence on cell surface membrane with all three HuMab anti-O8E antibodies at 0 min. In 30 min incubation, the membrane fluorescence intensity significantly decreased while staining increased inside of the cells. At the 120 min point, the fluorescence on the membrane disappeared and instead appeared to be present in intracellular compartments. The data demonstrates that HuMab anti-O8E antibodies can be specifically internalized upon binding to O8E-expressing endogenous tumor cells.

### Example 14

### Efficacy of Anti-O8E Antibodies on HEK-B7H4 Tumors in SCID Mice

In this Example, SCID mice implanted with HEK-B7H4 tumors are treated in vivo with naked anti-O8E antibodies to examine the in vivo effect of the antibodies on tumor growth.

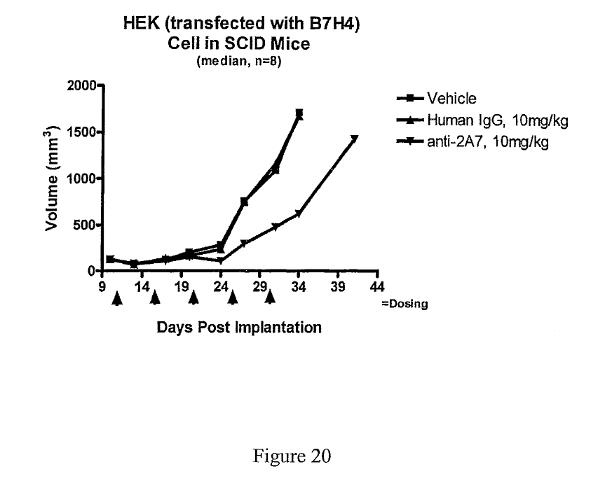

Severe combined immune deficient (SCID) mice, which lack functional B and T lymphocytes were used to study tumor growth. Cells from the HEK tumor cell line transfected with B7H4 were implanted subcutaneously at 5 million cells/mouse in matrigel (50% v/v). Each mouse received an inoculum of 0.2 ml of cells on day 0. The mice were checked for tumor growth starting at day 10 and monitored twice weekly for tumor growth for approximately 6 weeks. When tumors reached about 130 mm³, the mice were randomized by tumor volume into 3 groups. The mice were treated either with 10 mg/kg naked anti-O8E antibody 2A7, an isotype control antibody or formulation buffer as a negative control. The animals were dosed by intraperitoneal injection every 5 days for 5 injections. Using an electronic caliper, the tumors were measured three dimensionally (height×width×length) and tumor volume was calculated. Mice were euthanized when tumors reached a volume of 1500 mm³ or showed greater than 15% weight loss. The results are shown in FIG. 20. Tumor growth was inhibited by treatment with the anti-O8E antibody 2A7. The median tumor growth inhibition for the group treated with 2A7 was 63% on day 34. The tumors resumed growth after the dosing was stopped. These results show that anti-O8E antibodies are effective in treating tumors that express O8E in vivo.

### Example 15

### Preparation of B7H4 Antibody Drug Conjugate

The conjugation of B7H4 monoclonal antibody component and Toxin B was performed as follows. The antibody at ˜5 mg/ml in 100 mM Na-phosphate, 50 mM NaCl, 2 mM DTPA, pH 8.0, was thiolated with a 7-fold molar excess of 2-Iminothiolane. The thiolation reaction was allowed to proceed for 1 hour at room temperature with continuous mixing

### Conjugation to Toxin B:

Following thiolation, the antibody was buffer exchanged into conjugation buffer (50 HEPES, 5 mM Glycine, 0.5% Povidone (10K), 2 mM DTPA, pH 5.5) via a PD10 column (Sephadex G-25). The concentration of the thiolated antibody was determined at 280 nm. The thiol concentration was measured using the dithiodipyridine assay.

A 5 mM stock of MED-Toxin Bin DMSO was added at a 3-fold molar excess per thiol of antibody and mixed for 90 minutes at room temperature. Following conjugation, 100 mM N-ethylmaleimide in DMSO was added at a 10-fold molar excess of thiol per antibody to quench any unreacted thiols. This quenching reaction was done for one hour at room temperature with continuous mixing.

### Purification:

The B7H4 antibody drug conjugate was 0.2 μm filtered prior to Cation-exchange chromatographic purification. The SP Sepharose High Performance Cation Exchange column (CEX) was regenerated with 5 CV (column volume) of 50 mM HEPES, 5 mM Glycine, 0.5% Povidone, 1M NaCl, pH 5.5. Following regeneration, the column was equilibrated with 3 CVs of equilibration buffer (50 mM HEPES, 5 mM Glycine, 0.5% Povidone, pH 5.5). B7H4-Toxin B conjugate was loaded and the column was washed once with the equilibration buffer. The conjugate was eluted with 50 mM HEPES, 5 mM Glycine, 230 mM NaCl, 0.5% Povidone, pH 5.5. Eluate was collected in fractions. The column was then regenerated with 50 mM HEPES, 5 mM Glycine, 1M NaCl, 0.5% Povidone, pH 5.5 to remove protein aggregates and any unreacted MED Toxin B

Fractions containing monomeric antibody conjugate were pooled. Antibody conjugate concentration and substitution ratios were determined by measuring absorbance at 280 and 340 nm.

### Formulation

The purified CEX eluate pool was buffer exchanged into 50 mM HEPES, 5 mM Glycine, 100 mM NaCl, 0.5% Povidone, pH 6.0 by dialysis using a 10 MWCO membrane. Post-dialysis, antibody conjugate concentration and substitution ratios were determined by measuring absorbance at 280 and 340 nm.

### Example 16

### Efficacy of Antibody-Drug Conjugates on HEK-B7H4 Tumors in SCID Mice

A HEK293-B7H4 xenograft study was performed as follows. 5 million HEK293-B7H4 cells were implanted sub-cutaneously in SCID mice. Mice were assigned to treatment groups when tumors exceeded an average of 70 mm³ Mice were treated with 2A7-Toxin B, IgG control-Toxin B, or vehicle control with a single dose (0.1 umol/kg calculated for Toxin B) when tumors exceeded an average of 70 mm³ Mice were weighed and measured for tumors three dimensionally using an electronic caliper once weekly after implantation. Tumor volumes were calculated as height×width×length/2. This HEK293-B7H4 model expresses high levels of B7H4 on the cell surface. IgG control-Toxin B is used as an isotype control as the xenographs are negative for IgG.

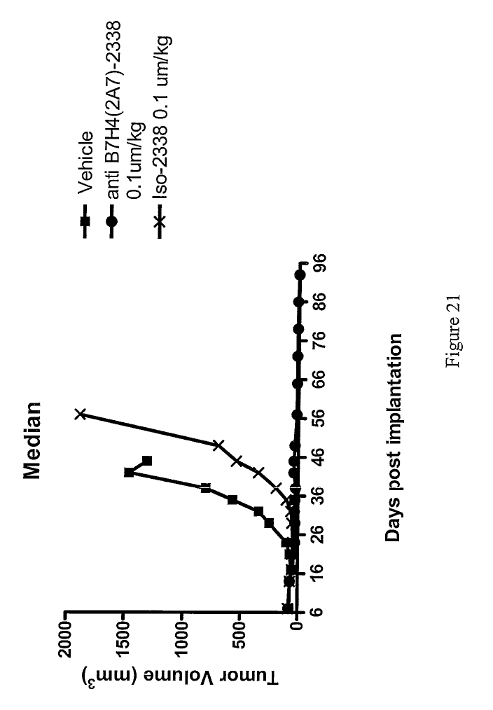

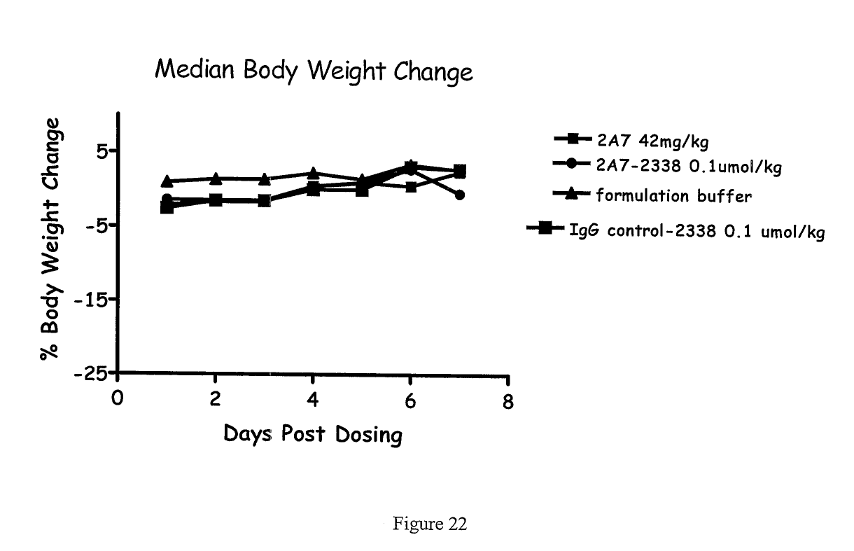

As illustrated in FIG. 21, while HEK293-B7H4 tumors grew well in the mice and there was no difference in tumor growth between the vehicle control and the toxin conjugated isotype control, treatment with 2A7-Toxin B resulted in complete tumor regression in all mice in this group. In contrast, FIG. 22 illustrates that there was no difference on body weights between the various groups. Accordingly, while targeting of B7H4 on tumors expressing this protein with 2A7-Toxin B causes complete tumor regression in this model, this study also shows no signs of target toxicity by administration of 2A7-Toxin B.

### Example 17

### Immunohistochemistry Using an Anti-O8E Antibody

The ability of the anti-B7H4 HuMAb 2A7 to recognize B7H4 by immunohistochemistry was examined using clinical biopsies from ovarian cancer, lung cancer, breast cancer, and head & neck cancer

For immunohistochemistry, 5 μm frozen sections were used (Ardais Inc, USA). After drying for 30 minutes, sections were fixed with acetone (at room temperature for 10 minutes) and air-dried for 5 minutes. Slides were rinsed in PBS and then pre-incubated with 10% normal goat serum in PBS for 20 min and subsequently incubated with 10 μg/ml fitcylated antibody in PBS with 10% normal goat serum for 30 min at room temperature. Next, slides were washed three times with PBS and incubated for 30 min with mouse anti-FITC (10 μg/ml DAKO) at room temperature. Slides were washed again with PBS and incubated with Goat anti-mouse HRP conjugate (DAKO) for 30 minutes at room temperature. Slides were washed again 3× with PBS. Diaminobenzidine (Sigma) was used as substrate, resulting in brown staining. After washing with distilled water, slides were counter-stained with hematoxyllin for 1 min. Subsequently, slides were washed for 10 secs in running distilled water and mounted in glycergel (DAKO). Clinical biopsy immunohistochemical staining displayed positive staining in the lung cancer, breast cancer, ovarian cancer, and head & neck cancer samples.

### Example 18

### Quantitative RT-PCR on Normal and Cancer Tissues

Various normal and cancerous tissue samples were screened for O8E mRNA expression using quantitative reverse transcriptase PCR (RT-PCR). Expression of mRNA is indicative of O8E protein expression.

For quantitative RT-PCR, the following O8E primers were used: B7-H4.3: AGGATGGAATCCTGAGCTGCACTT; B7-H4.4: TCCGACAGCTCATCTTTGCC-TTCT as provided by Operon (Huntsville, Ala.). Standard reaction conditions were used (5 μl cDNA template at 1 ng/μl, 0.1 μl upstream primer at 40 μM, 0.1 μl downstream primer at 40 μM, 6 μl 2×SYBR Green PCR mix (Applied Biosystems #4367659), and 0.8 μl water). The cDNA was amplified for 40 cycles using standard PCR conditions in an ABI Prism 7900HT (Applied Biosystems, Foster City, Calif.). The quantitative RT-PCR results are shown in Table 7 below. Samples with undetermined counts represent values that were below a fluorescence threshold. Breast, ovarian and head and neck tumors were shown to express O8E, with the highest levels of expression seen in some ovarian and head and neck cancer samples. This demonstrates that there is increased expression of O8E in breast, ovarian and head and neck tumor samples relative to normal tissue.

TABLE 7
Quantitative RT-PCR expression in normal and cancer tissues

| Tissue | Count | Quantity |
|---|---|---|
| N.Adipose (#301) | 28.953062 | 25.57793 |
| N.Artery (#303) | 31.856901 | 3.0423617 |
| N.Bladder (#257) | 30.620392 | 7.5326214 |
| N.Bone Marrow (#342) | Undetermined | 0 |
| N.Brain (#258) | 34.33955 | 0.49280354 |
| N.Breast (#259) | 25.63064 | 292.28528 |
| N.Colon (#261) | Undetermined | 0 |
| N.Esophagus (#262) | 32.27514 | 2.2388945 |
| N.Heart (#125) | Undetermined | 0 |
| N.Kidney (#264) | 33.599422 | 0.8479082 |
| N.Liver (#266) | Undetermined | 0 |
| N.Lung (#268) | 32.44523 | 1.9763907 |
| N.Lymph Node (#315) | Undetermined | 0 |
| N.Ovary (#270) | 35.045704 | 0.29364112 |
| N.Pancreas (#271) | 28.446985 | 37.06916 |
| N.Peripheral Blood | 34.652363 | 0.39180183 |
| Leukocytes (#302) | | |
| N.Prostate (#272) | 32.635994 | 1.7184163 |
| N.Retina (#256) | 34.70426 | 0.37717298 |
| N.Skeletal Muscle (#119) | Undetermined | 0 |
| N.Skeletal Muscle (#126) | Undetermined | 0 |
| N.Skin (#273) | Undetermined | 0 |
| N.Spinal Cord (#129) | 39.383526 | 0.01220525 |
| N.Spleen (#274) | Undetermined | 0 |
| N.Stomach (#275) | Undetermined | 0 |
| N.Tongue (#324) | 30.956758 | 5.886249 |
| N.Tonsil (#325) | Undetermined | 0 |
| N.Trachea (#314) | 29.771343 | 14.03797 |
| Breast T. (#176) | 33.798374 | 0.7328206 |
| Breast T. (#177) | 25.759022 | 266.02777 |
| Breast T. (#178) | 28.572468 | 33.81085 |
| Breast T. (#179) | 25.31508 | 368.374 |
| Breast T. (#180) | 29.323488 | 19.494516 |
| Head/Neck T. (Larynx, #402) | 28.116425 | 47.23582 |
| Head/Neck T. (Pharynx, #403) | 25.776083 | 262.72076 |
| Head/Neck T. (Tongue, #403) | 26.950275 | 111.07142 |
| Head/Neck T. (Tonsil, #404) | 23.03704 | 1957.3722 |
| Kidney T. (#167) | 27.029814 | 104.77927 |
| Ovary T. (#187) | 25.321087 | 366.75525 |
| Ovary T. (#188) | 22.846964 | 2250.0833 |
| Ovary T. (#189) | 25.079527 | 437.81958 |
| Ovary T. (#190) | 27.964441 | 52.80399 |
| Ovary T. (#191) | 22.686525 | 2530.9656 |

### SUMMARY OF SEQUENCE LISTING

| SEQ ID NO: | SEQUENCE |
|---|---|
| 1 | V_{H} a.a. 11G1 |
| 2 | V_{H} a.a. 2A7 |
| 3 | V_{H} a.a. 2F9 |
| 4 | V_{H} a.a. 12E1 |
| 5 | V_{H} a.a. 13D12 |
| 6 | V_{L} a.a. 11G1 |
| 7 | V_{L} a.a. 2A7 |
| 8 | V_{L} a.a. 2F9 |
| 9 | V_{L} a.a. 12E1 |
| 10 | V_{L} a.a. 13D12 |
| 11 | V_{H} CDR1 a.a. 11G1 |
| 12 | V_{H} CDR1 a.a. 2A7 |
| 13 | V_{H} CDR1 a.a. 2F9 |
| 14 | V_{H} CDR1 a.a. 12E1 |
| 15 | V_{H} CDR1 a.a. 13D12 |
| 16 | V_{H} CDR2 a.a. 11G1 |
| 17 | V_{H} CDR2 a.a. 2A7 |
| 18 | V_{H} CDR2 a.a. 2F9 |
| 19 | V_{H} CDR2 a.a. 12E1 |
| 20 | V_{H} CDR2 a.a. 13D12 |
| 21 | V_{H} CDR3 a.a. 11G1 |
| 22 | V_{H} CDR3 a.a. 2A7 |
| 23 | V_{H} CDR3 a.a. 2F9 |
| 24 | V_{H} CDR3 a.a. 12E1 |
| 25 | V_{H} CDR3 a.a. 13D12 |
| 26 | V_{L} CDR1 a.a. 11G1 |
| 27 | V_{L} CDR1 a.a. 2A7 |
| 28 | V_{L} CDR1 a.a. 2F9 |
| 29 | V_{L} CDR1 a.a. 12E1 |
| 30 | V_{L} CDR1 a.a. 13D12 |
| 31 | V_{L} CDR2 a.a. 11G1 |
| 32 | V_{L} CDR2 a.a. 2A7 |
| 33 | V_{L} CDR2 a.a. 2F9 |
| 34 | V_{L} CDR2 a.a. 12E1 |
| 35 | V_{L} CDR2 a.a. 13D12 |
| 36 | V_{L} CDR3 a.a. 11G1 |
| 37 | V_{L} CDR3 a.a. 2A7 |
| 38 | V_{L} CDR3 a.a. 2F9 |
| 39 | V_{L} CDR3 a.a. 12E1 |
| 40 | V_{L} CDR3 a.a. 13D12 |
| 41 | V_{H} n.t. 11G1 |
| 42 | V_{H} n.t. 2A7 |
| 43 | V_{H} n.t. 2F9 |
| 44 | V_{H} n.t. 12E1 |
| 45 | V_{H} n.t. 13D12 |
| 46 | V_{L} n.t. 11G1 |
| 47 | V_{L} n.t. 2A7 |
| 48 | V_{L} n.t. 2F9 |
| 49 | V_{L} n.t. 12E1 |
| 50 | V_{L} n.t. 13D12 |
| 51 | V_{H} 4-34 |
| 52 | V_{H} 3-53 |
| 53 | V_{H} 3-9/D3-10/JH6b |
| 54 | V_{K} A27 |
| 55 | V_{K} L6/JK1 |
| 56 | human B7-H4 |
| 57 | Peptide Linker |
| 58 | Peptide Linker |
| 59 | Peptide Linker |
| 60 | Peptide Linker |
| 61 | Peptide Linker |
| 62 | Peptide Linker |
| 63 | Peptide Linker |
| 64 | Peptide Linker |
| 65 | Peptide Linker |
| 66 | Peptide Linker |
| 67 | Peptide Linker |
| 68 | Peptide Linker |
| 69 | Peptide Linker |

1. An antibody-partner molecule conjugate comprising a human monoclonal antibody, or an antigen-binding portion thereof, wherein the antibody binds human B7-H4 and the antibody-partner molecule conjugates exhibits at least one of the following properties:
(a) binds to human B7-H4 with an affinity of 1×10⁻⁸M or less; or
(b) inhibits growth of B7-H4-expressing cells in vivo when conjugated to a cytotoxin.

2-3. (canceled)

4. An antibody-partner molecule conjugate comprising a monoclonal antibody, or antigen binding portion thereof, which binds an epitope on human B7-H4 recognized by a reference antibody, wherein the reference antibody comprises:
(a) a heavy chain variable region comprising the amino acid sequence of SEQ ID NO: 1 and a light chain variable region comprising the amino acid sequence of SEQ ID NO: 6;
(b) a heavy chain variable region comprising the amino acid sequence of SEQ ID NO: 2 and a light chain variable region comprising the amino acid sequence of SEQ ID NO: 7.
(c) a heavy chain variable region comprising the amino acid sequence of SEQ ID NO: 3 and a light chain variable region comprising the amino acid sequence of SEQ ID NO: 8;
(d) a heavy chain variable region comprising the amino acid sequence of SEQ ID NO: 4 and a light chain variable region comprising the amino acid sequence of SEQ ID NO: 9; or
(e) a heavy chain variable region comprising the amino acid sequence of SEQ ID NO: 5 and a light chain variable region comprising the amino acid sequence of SEQ ID NO: 10

5-9. (canceled)

10. The antibody-partner molecule conjugate of claim 1, which comprises:
(a) a heavy chain variable region CDR1 comprising SEQ ID NO: 11;
(b) a heavy chain variable region CDR2 comprising SEQ ID NO: 16;
(c) a heavy chain variable region CDR3 comprising SEQ ID NO: 21;
(d) a light chain variable region CDR1 comprising SEQ ID NO: 26;
(e) a light chain variable region CDR2 comprising SEQ ID NO: 31; and
(f) a light chain variable region CDR3 comprising SEQ ID NO: 36; or
(g) a heavy chain variable region CDR1 comprising SEQ ID NO: 12;
(h) a heavy chain variable region CDR2 comprising SEQ ID NO: 17;
(i) a heavy chain variable region CDR3 comprising SEQ ID NO: 22;
(j) a light chain variable region CDR1 comprising SEQ ID NO: 27;
(k) a light chain variable region CDR2 comprising SEQ ID NO: 32; and
(l) a light chain variable region CDR3 comprising SEQ ID NO: 37; or
(m) a heavy chain variable region CDR1 comprising SEQ ID NO: 13;
(n) a heavy chain variable region CDR2 comprising SEQ ID NO: 18;
(o) a heavy chain variable region CDR3 comprising SEQ ID NO: 23;
(p) a light chain variable region CDR1 comprising SEQ ID NO: 28;
(q) a light chain variable region CDR2 comprising SEQ ID NO: 33; and
(r) a light chain variable region CDR3 comprising SEQ ID NO: 38; or
(s) a heavy chain variable region CDR1 comprising SEQ ID NO: 14;
(t) a heavy chain variable region CDR2 comprising SEQ ID NO: 19;
(u) a heavy chain variable region CDR3 comprising SEQ ID NO: 24;
(v) a light chain variable region CDR1 comprising SEQ ID NO: 29;
(w) a light chain variable region CDR2 comprising SEQ ID NO: 34; and
(x) a light chain variable region CDR3 comprising SEQ ID NO: 39; or
(y) a heavy chain variable region CDR1 comprising SEQ ID NO: 15;
(z) a heavy chain variable region CDR2 comprising SEQ ID NO: 20;
(aa) a heavy chain variable region CDR3 comprising SEQ ID NO: 25;
(bb) a light chain variable region CDR1 comprising SEQ ID NO: 30;
(cc) a light chain variable region CDR2 comprising SEQ ID NO: 35; and
(dd) a light chain variable region CDR3 comprising SEQ ID NO: 40.

11-14. (canceled)

15. The antibody-partner molecule conjugate of claim 1, comprising:
(a) a heavy chain variable region comprising an amino acid sequence selected from the group consisting of SEQ ID NOs: 1-5; and
(b) a light chain variable region comprising an amino acid sequence selected from the group consisting of SEQ ID NOs: 6-10;
wherein the antibody specifically binds a human B7-H4 protein.

16-21. (canceled)

22. The antibody-partner molecule conjugate of claim 1, wherein the partner molecule is a therapeutic agent.

23. (canceled)

24. The antibody-partner molecule conjugate of claim 22, wherein the therapeutic agent is a cytotoxin.

25. (canceled)

26. The antibody-partner molecule conjugate of claim 22, wherein the therapeutic agent is a radioactive isotope.

27. The antibody-partner molecule conjugate of claim 1, wherein the conjugate is present in combination with a pharmaceutically acceptable carrier.

28-32. (canceled)

33. A method of treating cancer in a subject comprising administering to the subject an antibody-partner molecule conjugate of claim 1 such that the cancer is treated in the subject.

34-37. (canceled)

38. An antibody-partner molecule conjugate of claim 1, wherein the partner molecule is conjugated to the antibody by a chemical linker.

39. The antibody-partner molecule conjugate of claim 38 wherein the chemical linker is selected from the group consisting of peptidyl linkers, hydrazine linkers, and disulfide linkers.

40. The method of treating cancer in a subject of claim 33, wherein the treatment comprises inhibiting growth of a B7-H4-expressing tumor cell comprising contacting the B7-H4-expressing tumor cell with the antibody-partner molecule conjugate of claim 1 such that growth of the B7-H4-tumor cell is inhibited, and cancer is treated in the subject.

41. The method of claim 40, wherein the therapeutic agent is a cytotoxin.

42. The method of claim 40, wherein the B7-H4-expressing tumor cell is a prostate cancer or bladder cancer tumor cell.


SEQUENCE LISTING (76 sequences)

SEQ ID NO: 1 - protein, 119 aa, Homo sapiens
QVQLQQWGAGLLKPSETLSLTCAVYGGSFSDYFWTWIRQPPGKGLEWIGEINHSGTTNYNPSLKSRVTISADTSKNQFSLRLSSVTAADTAVYYCARLSSWSNWAFEYWGQGTLVTVSS

SEQ ID NO: 2 - protein, 115 aa, Homo sapiens
EVQLVESGGGLIQPGGSLRLSCAASGFTVSSNYMNWVRQAPGKGLEWVSVIYGSGRTYYADSVKGRVTISRDNSKNTLYLQMNSLRAEDTAVYYCARDTYAMDVWGQGTTVTVSS

SEQ ID NO: 3 - protein, 116 aa, Homo sapiens
EVQLVESGGGLIQPGGSLRLSCAASGFIVSRNYMNWVRQAPGKGLEWVSVIYGSGRTDCADSVKGRFTISRDNSKNTLYLQMNSLRAEDTAVYYCARDGDYGMDVWGQGTTVTVSS

SEQ ID NO: 4 - protein, 126 aa, Homo sapiens
EVQLVESGGGLVQPGRSLRLSCVASGFTFDDYAMHWVRQAPGKGLEWVSGISWNSGSIGYADSVKGRFTISRDNAKNSLYLQMNSLRAEDTALYYCTKALYGSGSSDFYYYGMDVWGQGTTVAVSS

SEQ ID NO: 5 - protein, 122 aa, Homo sapiens
QVQLQQWGAGLLKPSETLSLTCAVYGGSFSGYYWSWIRQPPGKGLEWIGKINHSGSTNYNPSLKSRVTISVDTSKNQFSLKLNSVTAADTAVYYCARELRYFENYYYGMDVWGQGTTVTVSS

SEQ ID NO: 6 - protein, 108 aa, Homo sapiens
EIVLTQFPGTLSLSPGERATLSCRASQSVSSTYLAWYQQKPGQAPRVLIYGASRRATGIPDRFSGSGSGTDFTLTISRLEPEDFAVYYCQQYGSSPLTFGGGTKVEIK

SEQ ID NO: 7 - protein, 109 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPMYTFGQGTKLEIK

SEQ ID NO: 8 - protein, 109 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLYTFGQGTKLEIK

SEQ ID NO: 9 - protein, 103 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRRTFGQGTKVEIK

SEQ ID NO: 10 - protein, 108 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPRTFGQGTKVEIK

SEQ ID NO: 11 - protein, 5 aa, Homo sapiens
DYFWT

SEQ ID NO: 12 - protein, 6 aa, Homo sapiens
SNYMNW

SEQ ID NO: 13 - protein, 5 aa, Homo sapiens
RNYMN

SEQ ID NO: 14 - protein, 5 aa, Homo sapiens
DYAMH

SEQ ID NO: 15 - protein, 5 aa, Homo sapiens
GYYWS

SEQ ID NO: 16 - protein, 16 aa, Homo sapiens
EINHSGTTNYNPSLKS

SEQ ID NO: 17 - protein, 16 aa, Homo sapiens
VIYGSGRTYYADSVKG

SEQ ID NO: 18 - protein, 16 aa, Homo sapiens
VIYGSGRTDCADSVKG

SEQ ID NO: 19 - protein, 17 aa, Homo sapiens
GISWNSGSIGYADSVKG

SEQ ID NO: 20 - protein, 16 aa, Homo sapiens
KINHSGSTNYNPSLKS

SEQ ID NO: 21 - protein, 11 aa, Homo sapiens
LSSWSNWAFEY

SEQ ID NO: 22 - protein, 7 aa, Homo sapiens
DTYAMDV

SEQ ID NO: 23 - protein, 8 aa, Homo sapiens
DGDYGMDV

SEQ ID NO: 24 - protein, 16 aa, Homo sapiens
LYGSGSSDFYYYGMDV

SEQ ID NO: 25 - protein, 14 aa, Homo sapiens
ELRYFENYYYGMDV

SEQ ID NO: 26 - protein, 12 aa, Homo sapiens
RASQSVSSTYLA

SEQ ID NO: 27 - protein, 12 aa, Homo sapiens
RASQSVSSSYLA

SEQ ID NO: 28 - protein, 12 aa, Homo sapiens
RASQSVSSSYLA

SEQ ID NO: 29 - protein, 11 aa, Homo sapiens
RASQSVSSYLA

SEQ ID NO: 30 - protein, 12 aa, Homo sapiens
RASQSVSSSYLA

SEQ ID NO: 31 - protein, 7 aa, Homo sapiens
GASRRAT

SEQ ID NO: 32 - protein, 7 aa, Homo sapiens
GASSRAT

SEQ ID NO: 33 - protein, 7 aa, Homo sapiens
GASSRAT

SEQ ID NO: 34 - protein, 7 aa, Homo sapiens
DASNRAT

SEQ ID NO: 35 - protein, 7 aa, Homo sapiens
GASSRAT

SEQ ID NO: 36 - protein, 9 aa, Homo sapiens
QQYGSSPLT

SEQ ID NO: 37 - protein, 10 aa, Homo sapiens
QQYGSSPMYT

SEQ ID NO: 38 - protein, 10 aa, Homo sapiens
QQYGSSPLYT

SEQ ID NO: 39 - protein, 5 aa, Homo sapiens
QQRRT

SEQ ID NO: 40 - protein, 9 aa, Homo sapiens
QQYGSSPRT

SEQ ID NO: 41 - DNA, 357 bp, Homo sapiens
caggtgcagctacagcagtggggcgcaggactgttgaagccttcggagaccctgtccctcacctgcgctgtctatggtgggtccttcagtgattacttctggacctggatccgccagcccccagggaagggcctggagtggattggggaaatcaatcatagtggaaccaccaactacaacccgtccctcaagagtcgagtcaccatttcagcagacacgtccaagaaccagttctccctgaggctgagctctgtgaccgccgcggacacggctgtgtattactgtgcgagactcagcagctggtcgaactgggcctttgagtactggggccagggaaccctggtcaccgtctcctca

SEQ ID NO: 42 - DNA, 345 bp, Homo sapiens
gaggtgcagctggtggagtctggaggaggcttgatccagcctggggggtccctgagactctcctgtgcagcctctgggttcaccgtcagtagcaactacatgaactgggtccgccaggctccagggaaggggctggagtgggtctcagttatttatggcagtggtagaacatattacgcagactccgtgaagggccgagtcaccatctccagagacaattccaagaacacgctgtatcttcaaatgaacagcctgagagccgaggacacggccgtgtattactgtgcgagagatacctacgctatggacgtctggggccaagggaccacggtcaccgtctcctct

SEQ ID NO: 43 - DNA, 348 bp, Homo sapiens
gaggtgcagttggtggagtctggaggaggcttgatccagcctggggggtccctgagactctcctgtgcagcctctgggttcatcgtcagtagaaactacatgaactgggtccgccaggctccagggaaggggctggagtgggtctcagttatttatggcagtggtaggacagactgcgcagactccgtgaagggccgattcaccatctccagagacaattccaagaacacgctgtatcttcaaatgaacagcctgagagccgaggacacggccgtgtattactgtgcgagagatggggactacggtatggacgtctggggccaagggaccacggtcaccgtctcctca

SEQ ID NO: 44 - DNA, 378 bp, Homo sapiens
gaagtgcagctggtggagtctgggggaggcttggtacagcctggcaggtccctgagactctcctgtgtagcctctggattcacctttgatgattatgccatgcactgggtccggcaagctccagggaagggcctggagtgggtctcaggtattagttggaatagtggtagcataggctatgcggactctgtgaagggccgattcaccatctccagagacaacgccaagaactccctgtatctgcaaatgaacagtctgagagctgaggacacggccttgtattactgtacaaaagccctctatggttcggggagttctgacttctactactacggtatggacgtctggggccaagggaccacggtcgccgtctcctca

SEQ ID NO: 45 - DNA, 366 bp, Homo sapiens
caggtgcagctacagcagtggggcgcaggactgttgaagccttcggagaccctgtccctcacctgcgctgtctatggtgggtccttcagtggttactactggagctggatccgccagcccccagggaaggggctggagtggattgggaaaatcaatcatagcggaagtaccaactacaacccgtccctcaagagtcgagtcaccatatcagtagacacgtccaagaaccagttctccctgaaactaaactctgtgaccgccgcggacacggctgtgtattactgtgcgagagaattacgatattttgaaaactactactacggtatggacgtctggggccaagggaccacggtcaccgtctcctca

SEQ ID NO: 46 - DNA, 324 bp, Homo sapiens
gaaattgtgttgacgcagtttccaggcaccctgtctttgtctccaggggaaagagccaccctctcctgcagggccagtcagagtgttagcagcacctacttagcctggtaccagcagaaacctggccaggctcccagggtcctcatctatggtgcatccagaagggccactggcatcccagacaggttcagtggcagtgggtctgggacagacttcactctcaccatcagcagactggagcctgaagattttgcagtgtattactgtcagcagtatggtagctcaccgctcactttcggcggagggaccaaggtggagatcaaa

SEQ ID NO: 47 - DNA, 327 bp, Homo sapiens
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacccatgtacacttttggccaggggaccaagctggagatcaaa

SEQ ID NO: 48 - DNA, 327 bp, Homo sapiens
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctctgtacacttttggccaggggaccaagctggagatcaaa

SEQ ID NO: 49 - DNA, 309 bp, Homo sapiens
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtaggacgttcggccaagggaccaaggtggaaatcaaa

SEQ ID NO: 50 - DNA, 324 bp, Homo sapiens
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctcggacgttcggccaagggaccaaggtggaaatcaaa

SEQ ID NO: 51 - protein, 97 aa, Homo sapiens
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCAR

SEQ ID NO: 52 - protein, 97 aa, Homo sapiens
EVQLVESGGGLIQPGGSLRLSCAASGFTVSSNYMSWVRQAPGKGLEWVSVIYSGGSTYYADSVKGRFTISRDNSKNTLYLQMNSLRAEDTAVYYCAR

SEQ ID NO: 53 - protein, 99 aa, Homo sapiens
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKD

SEQ ID NO: 54 - protein, 96 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSP

SEQ ID NO: 55 - protein, 94 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNW

SEQ ID NO: 56 - protein, 282 aa, Homo sapiens
MASLGQILFWSIISIIIILAGAIALIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASLCVSSFFAISWALLPLSPYLMLK

SEQ ID NO: 57 - protein, 4 aa, Homo sapiens
ALAL

SEQ ID NO: 58 - protein, 4 aa, Homo sapiens
  MOD_RES (1)..(1): Xaa is beta-alanine
XLAL

SEQ ID NO: 59 - protein, 4 aa, Homo sapiens
GFLG

SEQ ID NO: 60 - protein, 4 aa, Homo sapiens
LLGL

SEQ ID NO: 61 - protein, 4 aa, Homo sapiens
PRFK

SEQ ID NO: 62 - protein, 4 aa, Homo sapiens
TRLR

SEQ ID NO: 63 - protein, 4 aa, Homo sapiens
SKGR

SEQ ID NO: 64 - protein, 4 aa, Homo sapiens
PNDK

SEQ ID NO: 65 - protein, 6 aa, Homo sapiens
PVGLIG

SEQ ID NO: 66 - protein, 5 aa, Homo sapiens
GPLGV

SEQ ID NO: 67 - protein, 8 aa, Homo sapiens
GPLGIAGQ

SEQ ID NO: 68 - protein, 4 aa, Homo sapiens
PLGL

SEQ ID NO: 69 - protein, 8 aa, Homo sapiens
GPLGMLSQ

SEQ ID NO: 70 - protein, 8 aa, Homo sapiens
GPLGLWAQ

SEQ ID NO: 71 - protein, 22 aa, Homo sapiens
LSSWSNWAFEYWGQGTLVTVSS

SEQ ID NO: 72 - protein, 14 aa, Homo sapiens
ELRYFENYYYGMDV

SEQ ID NO: 73 - protein, 5 aa, Homo sapiens
YGSGS

SEQ ID NO: 74 - protein, 20 aa, Homo sapiens
YYYYYGMDVWGQGTTVTVSS

SEQ ID NO: 75 - protein, 11 aa, Homo sapiens
TFGQGTKVEIK

SEQ ID NO: 76 - protein, 12 aa, Homo sapiens
LTFGGGTKVEIK